(19) United States
(12) Patent Application Publication
(10) Pub. No.: US 2025/0368652 A1
(43) Pub. Date: Dec. 4, 2025

(54) MICROBIOCIDAL BICYCLIC HETEROCYCLIC CARBOXAMIDE DERIVATIVES

(71) Applicant: SYNGENTA CROP PROTECTION AG, Basel (CH)

(72) Inventors: Nicolas GERMAIN, Stein (CH); Andrew EDMUNDS, Stein (CH); Thomas James HOFFMAN, Stein (CH); Jagadeesh Prathap KILARU, Corlim, Ilhas (IN); Atul MAHAJAN, Corlim, Ilhas (IN); Ramya RAJAN, Corlim, Ilhas (IN)

(73) Assignee: SYNGENTA CROP PROTECTION AG, Basel (CH)

(21) Appl. No.: 18/877,846

(22) PCT Filed: Jun. 20, 2023
(86) PCT No.: PCT/EP2023/066659

§ 371 Date: Dec. 20, 2024

(30) Foreign Application Priority Data
Jun. 21, 2022 (IN) 202211035525
Oct. 4, 2022 (EP) 22199682.0

(51) Int. Cl. C07D 487/04; A01N 43/90; A01P 3/00

(52) U.S. Cl. CPC C07D 487/04; A01N 43/90; A01P 3/00

(57) ABSTRACT

A compound of formula (I) wherein the substituents are as defined in claim 1, useful as pesticides, especially as fungicides.

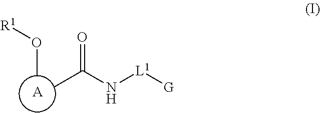

The present invention relates to unsaturated N-bridged bicyclic heterocyclic derivatives, e.g., as active ingredients, which have microbiocidal activity, and in particular, fungicidal activity. The invention also relates to agrochemical compositions which comprise at least one of the carboxamide derivatives, to processes of preparation of these compounds and to uses of the carboxamide derivatives or compositions in agriculture or horticulture for controlling or preventing infestation of plants, harvested food crops, seeds or non-living materials by phytopathogenic microorganisms, preferably fungi.

Many plant protection compounds have been developed to prevent or reduce plant disease caused by micro-organisms, for instance, by fungi. For example, WO 2021/233861 discloses azabicyclic(thio)amides as fungicidal compounds, and WO 2021/249995 discloses azabicyclyl-substituted heterocycles as fungicides. The azabicyclic compounds disclosed therein are C-bridged.

The present invention therefore provides, in a first aspect, compounds of formula (I)

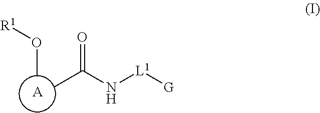

or agrochemically acceptable salt, stereoisomer, enantiomer, and N-oxide of the compound of formula (I), wherein:
R¹ is phenyl unsubstituted or substituted with 1, 2 or 3 independently selected substituents R¹¹; or
R¹ is a 5- or 6-membered monocyclic heteroaryl ring comprising 1, 2 or 3 heteroatoms each independently selected from N, O and S, wherein said heteroaryl ring is unsubstituted or substituted with 1 or 2 independently selected substituents R¹¹;
R¹¹ is hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, or cyclopropyloxy;
L¹ represents a direct bond, —O—, or —O—C(R^{L1A})(R^{L1B}) wherein R^{L1A} and R^{L1B} are independently selected from hydrogen and methyl; or R^{L1A} and R^{L1B} together with the carbon atom to which they are attached, form a cyclopropyl; or
L¹ represents —NR¹⁰—(CR²R³)ₘ— wherein R¹⁰ is selected from hydrogen or methyl; m=0 or 1, or
L¹ represents

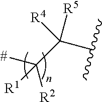

wherein # marks the bond to the nitrogen atom and the staggered line marks the bond to the group G;
R² and R³ are independently selected from hydrogen and methyl; and n is 0 or 1;
R⁴ and R⁵ are independently selected from hydrogen, hydroxy, fluoro, methyl, cyano, or methoxy; or R⁴ and R⁵ together with the carbon atom to which they are attached, form a carbonyl, cyclopropyl or cyclobutyl group;
G is selected from G-1, G-2, G-3, or G-4, wherein:
G-1 is phenyl or phenoxy, wherein said phenyl or phenoxy is unsubstituted or substituted with 1, 2, or 3 independently selected substituents R^{G1}
G-2 is a 5- or 6-membered monocyclic heteroaryl or heteroaryl-oxy; wherein said heteroaryl comprises 1, 2 or 3 heteroatoms each independently selected from N, O and S; and wherein said heteroaryl is unsubstituted or substituted with 1 or 2 independently selected substituents R^{G2};
G-3 is a 9- or 10-membered heterobicyclic ring system comprising 1, 2 or 3 heteroatoms each independently selected from N, O and S; wherein said heterobicyclic ring system is saturated, partially unsaturated, or aromatic; and wherein said heterobicyclic ring system is unsubstituted or substituted with 1 or 2 independently selected substituents R^{G3};
G-4 is a 9- or 10-membered carbobicyclic ring system; wherein said carbobicyclic ring system is saturated, partially unsaturated, or aromatic; and wherein said carbobicyclic ring system is unsubstituted or substituted with 1 or 2 independently selected substituents R^{G4}
R^{G1}, R^{G2}, R^{G3}, and R^{G4} are independently hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, or cyclopropyloxy;
A is selected from A-1 to A-17:

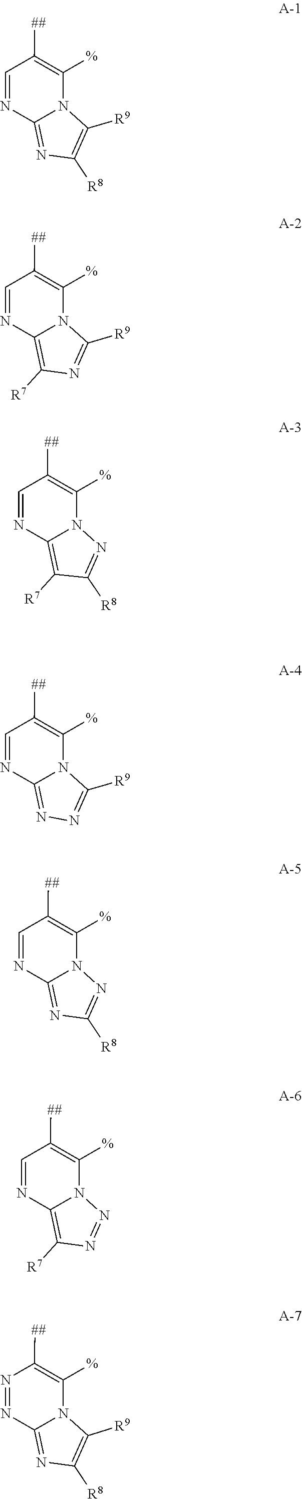

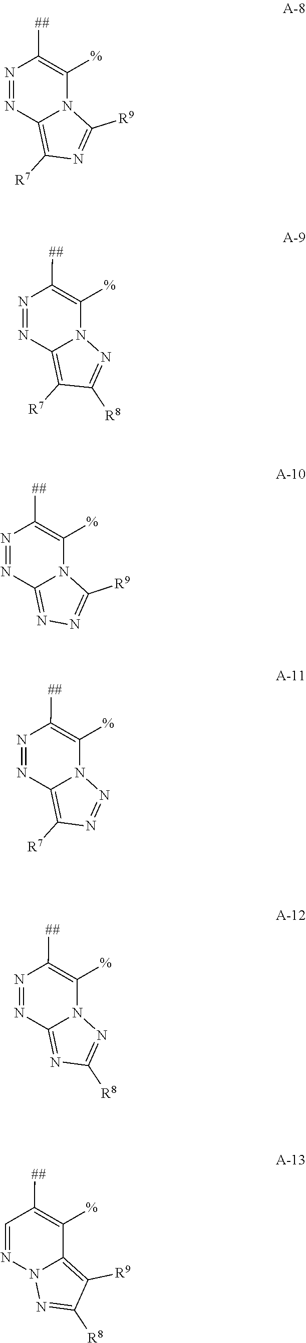

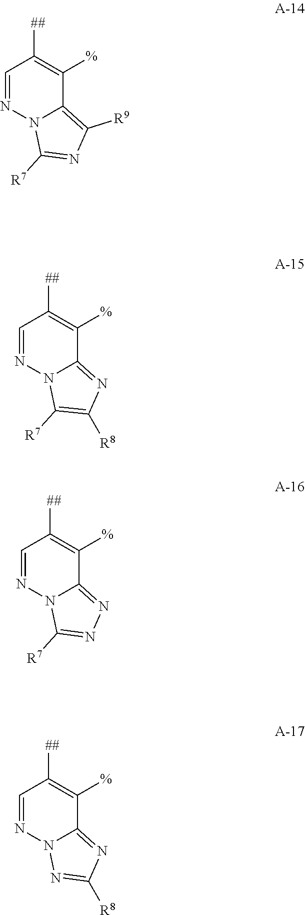

R⁷, R⁸, R⁹ are independently selected from hydrogen, fluoro, chloro, bromo, iodo, methyl, ethyl, propyl, isopropyl, allyl, propargyl, cyclopropylmethyl, cyclopentyl, cyclohexyl, —C(═O)OCH₃, —C(═O)N(CH₃)₂, 2-(dimethylamino)-2-oxo-ethyl, 2-(methylamino)-2-oxo-ethyl, difluoromethyl, trifluoromethyl, methylsulfonyl, methylsulfanyl, methoxy, ethoxy, cyano, hydroxyl, mercapto, or amino.

The present invention also provides a method of preparation of compounds of formula (I) as well as intermediate compounds useful in the preparation of compounds of formula (I).

Surprisingly, it has been found that the novel compounds of Formula (I) have, for practical purposes, a very advantageous level of biological activity for protecting plants against diseases that are caused by fungi.

According to a second aspect of the invention, there is provided an agrochemical composition comprising a fungicidally effective amount of a compound of Formula (I). Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of Formula (I), or a composition comprising this compound as active ingredient, is applied to the plants, to parts thereof or the locus thereof.

According to a fourth aspect of the invention, there is provided the use of a compound of Formula (I) as a fungicide. According to this particular aspect of the invention, the use may exclude methods for the treatment of the human or animal body by surgery or therapy.

According to a fifth aspect, the present invention makes available a plant propagation material, such as a seed, comprising, or treated with or adhered thereto, a compound of formula (I) or a composition comprising such a compound.

As used herein, the term “hydroxyl” or “hydroxy” means an —OH group.

As used herein, the term “mercapto” means an —SH group.

As used herein, the term “cyano” means a —CN group.

As used herein, amino means an —NH₂ group.

As used herein, nitro means an —NO₂ group.

As used herein, oxo means an ═0 group (eg, as in a carbonyl (C═O) group).

As used herein, the term “halogen” or “halo” refers to fluorine (fluoro), chlorine (chloro), bromine (bromo) or iodine (iodo), preferably fluorine, chlorine or bromine. This also applies, correspondingly, to halogen in combination with other meanings, such as haloalkyl.

As used herein, the term “C₁₋₄alkyl” refers to a straight or branched hydrocarbon chain radical consisting solely of carbon and hydrogen atoms, containing no unsaturation, having from one to four carbon atoms, and which is attached to the rest of the molecule by a single bond. C₁₋₃ alkyl should be construed accordingly. Examples of C₁₋₄alkyl include, but are not limited to, methyl, ethyl, iso-propyl.

As used herein, the term “C₂₋₃ alkenyl” refers to a straight or branched hydrocarbon chain radical group consisting solely of carbon and hydrogen atoms, containing at least one double bond that may be of either the (E) or (Z) configuration, having two or three carbon atoms, which is attached to the rest of the molecule by a single bond. Examples of C₂₋₃ alkenyl include, but are not limited to, vinyl (ethenyl), prop-1-enyl, allyl (prop-2-enyl).

As used herein, the term “C₂₋₃ alkynyl” refers to a straight or branched hydrocarbon chain radical group consisting solely of carbon and hydrogen atoms, containing at least one triple bond, having from two or three carbon atoms, and which is attached to the rest of the molecule by a single bond. Examples of C₂₋₃alkynyl include, but are not limited to, prop-1-ynyl and propargyl (prop-2-ynyl).

As used herein, the terms “C₁₋₄ haloalkyl”, “C₂₋₃ haloalkenyl”, and “C₂₋₃ haloalkynyl” refer respectively to a C₁₋₄ alkyl, C₂₋₃ alkenyl, and C₂₋₃ alkynyl radical as defined above, substituted by one or more of the same or different halogen atoms. Examples of C₁₋₄ haloalkyl include, but are not limited to fluoromethyl, fluoroethyl, difluoromethyl, trifluoromethyl, and 2,2,2-trifluoroethyl.

As used herein, the term “C₁₋₃ fluoroalkyl” refers to a C₁₋₃ alkyl radical as generally defined above substituted by one or more fluorine atoms. Examples of C₁₋₃ fluoroalkyl include, but are not limited to difluoromethyl and trifluoromethyl.

As used herein, the term “C₁₋₃ alkoxy” refers to a radical of the formula RₐO— where Rₐ is a C₁₋₃ alkyl radical as generally defined above. Examples of C₁₋₃ alkoxy include, but are not limited to, methoxy, ethoxy, iso-propoxy.

As used herein, the term “C₁₋₃ fluoroalkoxy” refers to a C₁₋₃ alkoxy radical as generally defined above substituted by one or more fluorine atoms. Examples of C₁₋₃ fluoroalkoxy include, but are not limited to trifluoromethoxy.

As used herein, the term “C₃₋₄ cycloalkyl” refers to a stable, monocyclic ring radical which is saturated and contains 3 or 4 carbon atoms.

As used herein, the term “C₁₋₃ alkylsulfanyl” refers to a radical of the formula —SRₐ wherein Rₐ is a C₁₋₃ alkyl radical as generally defined above.

As used herein, the term “C₁₋₃ alkylsulfonyl” refers to a radical of the formula —S(O)₂Rₐ wherein Rₐ is a C₁₋₃ alkyl radical as generally defined above.

The term “heteroaryl” as used herein refers to a 5- or 6-membered aromatic monocyclic ring having 1 to 3 heteroatoms independently selected from N, O and S. Examples of heteroaryls include J-1 to J-43 shown in Table J below. The staggered line in heteroaryls J-1 to J-43 represents the point of attachment to the rest of the compound. Preferred heteroaryls include pyridinyl, pyrimidinyl, pyridazinyl, pyrazinyl, and thiazolyl; preferably pyridinyl, and thiazolyl.

TABLE J
Heteroaryl J-1 to J-43:

| | |
|---|---|
| 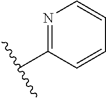 [SMILES: *c1ccccn1] | J-1 |
| 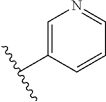 [SMILES: *c1cccnc1] | J-2 |
| 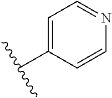 [SMILES: *c1ccncc1] | J-3 |
| 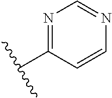 [SMILES: *c1ccncn1] | J-4 |
| 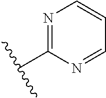 [SMILES: *c1ncccn1] | J-5 |
| 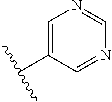 [SMILES: *c1cncnc1] | J-6 |
| 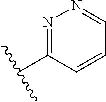 [SMILES: *c1cccnn1] | J-7 |
| 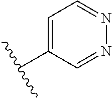 [SMILES: *c1ccnnc1] | J-8 |
| 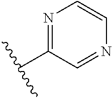 [SMILES: *c1cnccn1] | J-9 |
| 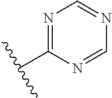 [SMILES: *c1ncncn1] | J-10 |
| 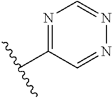 [SMILES: *c1cnncn1] | J-11 |
| 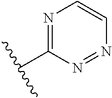 [SMILES: *c1nccnn1] | J-12 |
| 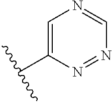 [SMILES: *c1cncnn1] | J-13 |
| 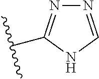 [SMILES: *c1nnc[nH]1] | J-14 |
| 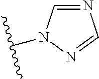 [SMILES: *n1cncn1] | J-15 |
| 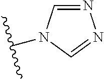 [SMILES: *n1cnnc1] | J-16 |
| 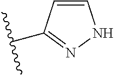 [SMILES: *c1cc[nH]n1] | J-17 |
| 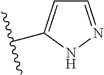 [SMILES: *c1ccn[nH]1] | J-18 |
| 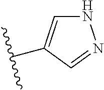 [SMILES: *c1cn[nH]c1] | J-19 |
| 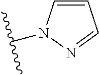 [SMILES: *n1cccn1] | J-20 |
| 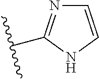 [SMILES: *c1ncc[nH]1] | J-21 |
| 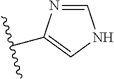 [SMILES: *c1c[nH]cn1] | J-22 |
| 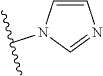 [SMILES: *n1ccnc1] | J-23 |
| 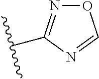 [SMILES: *c1ncon1] | J-24 |
| 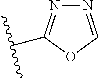 [SMILES: *c1nnco1] | J-25 |
| 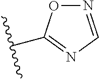 [SMILES: *c1ncno1] | J-26 |
| 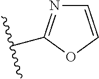 [SMILES: *c1ncco1] | J-27 |
| 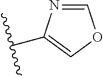 [SMILES: *c1cocn1] | J-28 |
| 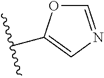 [SMILES: *c1cnco1] | J-29 |
| 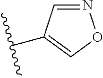 [SMILES: *c1cnoc1] | J-30 |
| 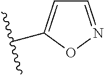 [SMILES: *c1ccno1] | J-31 |
| 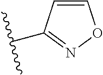 [SMILES: *c1ccon1] | J-32 |
| 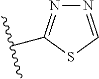 [SMILES: *c1nncs1] | J-33 |
| 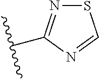 [SMILES: *c1ncsn1] | J-34 |
| 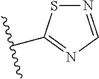 [SMILES: *c1ncns1] | J-35 |
| 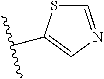 [SMILES: *c1cncs1] | J-36 |
| 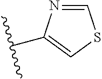 [SMILES: *c1cscn1] | J-37 |
| 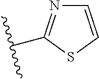 [SMILES: *c1nccs1] | J-38 |
| 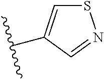 [SMILES: *c1cnsc1] | J-39 |
| 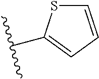 [SMILES: *c1cccs1] | J-40 |
| 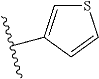 [SMILES: *c1ccsc1] | J-41 |
| 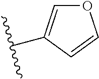 [SMILES: *c1ccoc1] | J-42 |
| 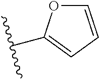 [SMILES: *c1ccco1] | J-43 |

The term “heterocyclyl” as used herein refers to a 3-, 4-, 5-, and 6-membered saturated monocyclic rings having 1 or 2 heteroatoms independently selected from nitrogen and oxygen. Examples of heterocyclyls include K-1 to K-26 shown in Table K below. The staggered line in heterocyclyls K-1 to K-26 represents the point of attachment to the rest of the compound. Some of the heterocyclyls shown below contain an asymmetric carbon, which means that compounds containing them may occur in chiral isomeric forms, i.e., enantiomeric or diastereomeric forms. Preferred heterocyclyls include pyrrolidinyl, piperidinyl, piperazinyl, and tetrahydropyranyl; preferably pyrrolidinyl, piperazinyl, and tetra hydropyranyl.

TABLE K
heterocyclyl K-1 to K-26:

| | |
|---|---|
| 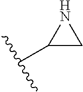 [SMILES: *C1CN1] | K-1 |
| 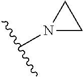 [SMILES: *N1CC1] | K-2 |
| 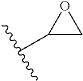 [SMILES: *C1CO1] | K-3 |
| 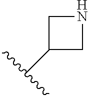 [SMILES: *C1CNC1] | K-4 |
| 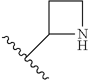 [SMILES: *C1CCN1] | K-5 |
| 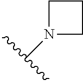 [SMILES: *N1CCC1] | K-6 |
| 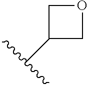 [SMILES: *C1COC1] | K-7 |
| 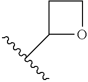 [SMILES: *C1CCO1] | K-8 |
| 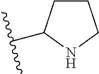 [SMILES: *C1CCCN1] | K-9 |
| 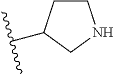 [SMILES: *C1CCNC1] | K-10 |
| 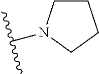 [SMILES: *N1CCCC1] | K-11 |
| 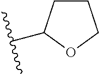 [SMILES: *C1CCCO1] | K-12 |
| 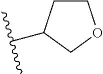 [SMILES: *C1CCOC1] | K-13 |
| 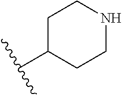 [SMILES: *C1CCNCC1] | K-14 |
| 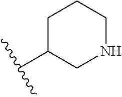 [SMILES: *C1CCCNC1] | K-15 |
| 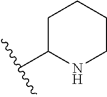 [SMILES: *C1CCCCN1] | K-16 |
| 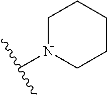 [SMILES: *N1CCCCC1] | K-17 |
| 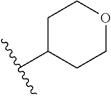 [SMILES: *C1CCOCC1] | K-18 |
| 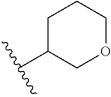 [SMILES: *C1CCCOC1] | K-19 |
| 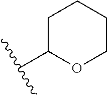 [SMILES: *C1CCCCO1] | K-20 |
| 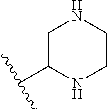 [SMILES: *C1CNCCN1] | K-21 |
| 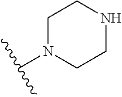 [SMILES: *N1CCNCC1] | K-22 |
| 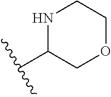 [SMILES: *C1COCCN1] | K-23 |
| 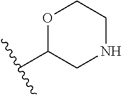 [SMILES: *C1CNCCO1] | K-24 |
| 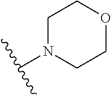 [SMILES: *N1CCOCC1] | K-25 |
| 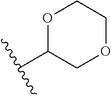 [SMILES: *C1COCCO1] | K-26 |

The term “optionally substituted” as used herein means that the group referenced is either unsubstituted or is substituted by a designated substituent, for example, “C₃-C₄ cycloalkyl is optionally substituted with 1 or 2 halo atoms” means C₃-C₄ cycloalkyl, C₃-C₄ cycloalkyl substituted with 1 halo atom and C₃-C₄ cycloalkyl substituted with 2 halo atoms. Further the term “optionally substituted” as used herein, means that the referred group is unsubstituted or substituted. The term “optionally substituted” can be used interchangeably with “unsubstituted or substituted”.

The term “optionally in the presence of” as used herein means, that a certain process is carried out in the presence or in the absence of the reagent referenced, for example “the process is carried out optionally in the presence of a base” means the process can be carried out with a base or without a base.

The staggered line as used herein, for example in heteroaryls shown in Table J and heterocyclyls shown in Table K, represent the point of connection/attachment to the rest of the compound.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, using known techniques and by observing results obtained under analogous circumstances. In determining the effective amount, a number of factors are considered including, but not limited to the type of plant or derived product to be applied; the pest to be controlled & its lifecycle; the particular compound applied; the type of application; and other relevant circumstances.

As used herein, the term “room temperature” or “RT” or “rt” refer to a temperature of about 15° C. to about 35° C. For example, rt can refer to a temperature of about 20° C. to about 30° C.

The presence of one or more possible asymmetric carbon atoms in a compound of formula (I) means that the compounds may occur in chiral isomeric forms, i.e., enantiomeric or diastereomeric forms. Also, atropisomers may occur as a result of restricted rotation about a single bond. formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I).

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di-ortrihydroxy-lower-alkylamine, for example mono-, di- or triethanolamine.

The compounds of formula (I) according to the invention also include hydrates which may be formed during the salt formation.

In each case, the compounds of formula (I) according to the invention are in free form, in oxidized form as an N-oxide, in covalently hydrated form, or in salt form, e.g., an agronomically usable or agrochemically acceptable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

Throughout the description,

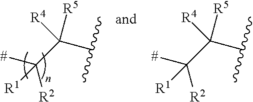

may also be written as —(CR²R³)ₙ—CR⁴R⁵— and —CR²R³—CR⁴R⁵—, respectively.

The following lists provide definitions, including preferred definitions, for substituents A, G, L¹, R¹, R², R³, R⁴, R⁵, R⁷, R¹¹, R^{G1}, R^{G2}, R^{G3}, and R^{G4} with reference to the compounds of formula (I) of the present invention. For any one of these substituents, any of the definitions given below may be combined with any definition of any other substituent given below or elsewhere in this document.

In an embodiment of each aspect of the invention, L¹ is
A. a direct bond, —NR¹⁰—CR²R³—, or —CR²R³—CR⁴R⁵— wherein R¹⁰ is selected from hydrogen and methyl; R² and R³ are independently selected from hydrogen and methyl; R⁴ and R⁵ are independently selected from hydrogen, hydroxy, fluoro, methyl, cyano, and methoxy; or R⁴ and R⁵ together with the carbon atom to which they are attached, form a carbonyl, cyclopropyl or cyclobutyl group; or
B. —NR¹⁰—CR²R³—, wherein R¹⁰ is selected from hydrogen and methyl; and R² and R³ are independently selected from hydrogen and methyl; or
C. —NR¹⁰—CR²R³—, wherein R¹⁰ is selected from hydrogen and methyl; and R² and R³ are both hydrogen; or
D. —NR¹⁰—CR²R³—, wherein R¹⁰ is hydrogen; and R² and R³ are both hydrogen; or
E. —CR²R³—CR⁴R⁵—, wherein R² and R³ are independently selected from hydrogen and methyl; R⁴ and R⁵ are independently selected from hydrogen, hydroxy, fluoro, methyl, cyano, hydroxy, and methoxy; or R⁴ and R⁵, together with the carbon atom to which they are attached, from a carbonyl, cyclopropyl or cyclobutyl group; or
F. —CR²R³—CR⁴R⁵—, wherein R² and R³ are hydrogen; R⁴ and R⁵ are independently selected from hydrogen and fluoro; or R⁴ and R⁵, together with the carbon atom to which they are attached, form a cyclopropyl or;
G. —CR²R³—CR⁴R⁵—, wherein R² and R³ are hydrogen; R⁴ and R⁵ are independently selected from hydrogen and fluoro; or
H. —CH₂—CHF— or —CH₂—CH₂—; or
I. a direct bond.

In an embodiment of each aspect of the invention, R¹ is:
A. phenyl unsubstituted or substituted with a one, two or three independently selected substituents R¹¹; for instance, one or two independently selected substituents R¹¹; preferably one substituent R¹¹; wherein R¹¹ is selected from hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, and cyclopropyloxy; or
B. phenyl substituted with one or two substituents R¹¹, for instance one substituent R¹¹; wherein R¹¹ is selected from hydroxyl, halogen, cyano, methyl, vinyl, ethynyl difluoromethyl, trifluoromethyl, methoxy, ethoxy, allyloxy, propargyloxy, difluoromethoxy, trifluoromethoxy, cyclopropyl, and cyclobutyl; or
C. phenyl substituted with one or two substituents, for instance one substituent, independently selected from hydroxyl, halogen, cyano, methyl, vinyl, ethynyl methoxy, ethoxy, allyloxy, propargyloxy, difluoromethoxy, trifluoromethoxy, cyclopropyl, and cyclobutyl; or
D. phenyl unsubstituted or substituted with a one or two substituents, for instance one substituent, independently selected from chloro, fluoro, cyano, methyl, methoxy, and cyclopropyl; or
E. phenyl substituted with a single substituent selected from methyl and cyclopropyl; or
F. cyclopropylphenyl, such as 3-cyclopropylphenyl.

In an embodiment of each aspect of the invention, R¹ may also be:
A. a 5- or 6-membered monocyclic heteroaryl ring comprising 1, 2 or 3 heteroatoms which may be the same or different, independently selected from N, O and S, wherein said heteroaryl ring is unsubstituted or substituted with 1 or 2 independently selected substituents R¹¹; or
B. a 6-membered monocyclic heteroaryl ring comprising 1, 2 or 3 nitrogen atoms, wherein said heteroaryl ring is unsubstituted or substituted with 1 or 2 independently selected substituents R¹¹; or
C. pyridine, pyrimidine, pyridazine, or 1,2,4-triazine, wherein any of said pyridine, pyrimidine, pyridazine, or 1,2,4-triazine is unsubstituted or substituted with one or two substituents, independently selected from hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, and cyclopropyloxy; or
D. pyridine substituted with a one or two substituents, independently selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
E. pyridine substituted with a single substituent selected from chloro, fluoro, cyano, methyl, methoxy, difluoromethoxy, and cyclopropyl; or
F. pyridine substituted with a single substituent selected from chloro, cyano, and methyl.

In an embodiment of each aspect of the invention, R¹ is:
A. phenyl, pyridyl, pyrimidyl, or pyridazinyl, each unsubstituted or substituted with a one or two independently selected substituents R¹¹; wherein R¹¹ is selected from hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, and cyclopropyloxy; or
B. phenyl, pyridyl, or pyrimidyl, each unsubstituted or substituted with a one or two independently selected substituents R¹¹; wherein R¹¹ is selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
C. phenyl, or pyridyl, each unsubstituted or substituted with a one or two independently selected substituents R¹¹; wherein R¹¹ is selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
D. phenyl, or pyridyl, each unsubstituted or substituted with a one or two independently selected substituents R¹¹; wherein R¹¹ is selected from chloro, fluoro, cyano, methyl, methoxy, difluoromethoxy, and cyclopropyl; or
E. phenyl, or pyridyl, each unsubstituted or substituted with a one or two independently selected substituents R¹¹; wherein R¹¹ is selected from chloro, cyano, methyl, and cyclopropyl; or
F. phenyl, or pyridyl, each unsubstituted or substituted with a one or two independently selected substituents R¹¹; wherein R¹¹ is selected from chloro, cyano and cyclopropyl.

In an embodiment of each aspect of the invention, R¹¹ is selected from:
A. hydroxyl, halogen, mercapto, amino, cyano, methyl, ethyl, propyl, isopropyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, and cyclopropyloxy; or
B. hydroxyl, halogen, cyano, methyl, vinyl, ethynyl, difluoromethyl, trifluoromethyl, methoxy, ethoxy, allyloxy, propargyloxy, difluoromethoxy, trifluoromethoxy, cyclopropyl, and cyclobutyl; or
C. hydroxyl, cyano, methyl, vinyl, difluoromethyl, trifluoromethyl, methoxy, ethoxy, allyloxy, propargyloxy, difluoromethoxy, trifluoromethoxy, cyclopropyl, and cyclobutyl; or
D. halogen, cyano, methyl, ethynyl, difluoromethyl, trifluoromethyl, methoxy, allyloxy, difluoromethoxy, trifluoromethoxy, and cyclopropyl; or
E. cyano, methyl, methoxy, allyloxy, trifluoromethoxy, cyclopropyl, and cyclobutyl; or
F. chloro, fluoro, cyano, methoxy, allyloxy, trifluoromethoxy, cyclopropyl, and cyclobutyl; or
G. halogen, cyano, methoxy, allyloxy, trifluoromethoxy, and cyclopropyl; or
H. chloro, fluoro, cyano, cyclopropyl and cyclobutyl; or
I. chloro, cyano, cyclopropyl and methyl.

In an embodiment of each aspect of the invention, G is G-1: phenyl or phenoxy, wherein said phenyl or phenoxy is unsubstituted or substituted with 1, 2 or 3 independently selected substituents R^{G1}. For instance, G-1 is:
A. phenyl unsubstituted or substituted with a one or two substituents, independently selected from hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, or cyclopropyloxy; or
B. phenyl unsubstituted or substituted with a one or two substituents, independently selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
C. phenyl unsubstituted or substituted with 1, 2 or 3 substituents, for instance 2 or 3 substituents, each independently selected from chloro, fluoro, methyl, and methoxy; or
D. phenyl substituted with one or two substituents, for instance one substituent, selected from chloro and methyl; or
E. phenyl substituted with two substituents independently selected from chloro and methyl; or
F. 2,4-disubstituted phenyl, each substituent independently selected from chloro and methyl; or
G. 2,4-dichlorophenyl.

In an embodiment of each aspect of the invention, G is G-2: a 5- or 6-membered monocyclic heteroaryl or heteroaryl-oxy; wherein said heteroaryl comprises 1, 2 or 3 heteroatoms each independently selected from N, O and S; and wherein said heteroaryl is unsubstituted or substituted with 1 or 2 independently selected substituents R^{G2}. For instance, G-2 is:
A. pyridine, pyrimidine, pyridazine, or 1,2,4-triazine wherein said pyridine, pyrimidine, pyridazine, 1,2,4-triazine is unsubstituted or substituted with one or two substituents, independently selected from hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, and cyclopropyloxy; or
B. pyridine, pyrimidine, or pyridazine, substituted with one or two substituents, independently selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
C. pyrimidine, or pyridazine, substituted with one or two substituents, independently selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
D. pyridine substituted with 1, 2 or 3 substituents, for instance 2 or 3 substituents each independently selected from chloro, fluoro, cyano, methyl, and methoxy; or
E. pyridine substituted with one or two substituents, for instance one substituent, selected from chloro and methyl.

In an embodiment of each aspect of the invention, G is G-3: a 9- or 10-membered heterobicyclic ring system comprising 1, 2 or 3 heteroatoms each independently selected from N, O and S; wherein said heterobicyclic ring system is saturated, partially unsaturated, or aromatic; and wherein said heterobicyclic ring system is unsubstituted or substituted with 1 or 2 independently selected substituents R^{G3}. For instance, G-3 is:
A. chroman-4-yl, isochroman-4-yl, 4H-chromen-4-yl, 2,3-dihydrobenzofuran-2-yl, 2,3-dihydrobenzofuran-3-yl, 1,3-benzodioxol-5-yl, benzothiazol-2-yl, benzothiazol-5-yl, benzothiazol-6-yl, benzooxazol-2-yl, benzooxazol-5-yl, benzooxazol-6-yl, benzofuran-2-yl, benzofuran-3-yl, benzofuran-5-yl, benzofuran-6-yl, benzothiophen-2-yl, benzothiophen-3-yl, benzothiophen-5-yl, benzothiophen-6-yl unsubstituted or substituted with 1, 2 or 3 substituents, for instance one or two substituents, each independently selected from chloro, fluoro, cyano, methyl, and methoxy; or
B. chroman-4-yl, isochroman-4-yl, 4H-chromen-4-yl, 2,3-dihydrobenzofuran-2-yl, 2,3-dihydrobenzofuran-3-yl, 1,3-benzodioxol-5-yl, benzothiazol-2-yl, benzothiazol-5-yl, benzothiazol-6-yl, benzooxazol-2-yl, benzofuran-2-yl, benzofuran-3-yl, or benzothiophen-2-yl, benzothiophen-3-yl substituted with one substituent selected from chloro, fluoro, cyano, methyl, and methoxy; or
C. chroman-4-yl, isochroman-4-yl, 4H-chromen-4-yl, 2,3-dihydrobenzofuran-2-yl, or 2,3-dihydrobenzofuran-3-yl substituted with one substituent selected from chloro, fluoro, cyano, methyl, and methoxy; or
D. 1,3-benzodioxol-5-yl, benzothiazol-2-yl, benzothiazol-5-yl, benzothiazol-6-yl, benzooxazol-2-yl, benzofuran-2-yl, benzofuran-3-yl, benzothiophen-2-yl, or benzothiophen-3-yl.

In an embodiment of each aspect of the invention, G is G-4: a 9- or 10-membered carbobicyclic ring system; wherein said carbobicyclic ring system is saturated, partially unsaturated, or aromatic; and wherein said carbobicyclic ring system is unsubstituted or substituted with 1 or 2 independently selected substituents R^{G4}. For instance, G-4 is:
A. naphthalen-2-yl, tetralin-1-yl, tetralin-2-yl, tetralin-6-yl, indan-1-yl, indan-2-yl, or indan-5-yl optionally substituted with 1, 2 or 3 substituents, for instance one or two substituents, each independently selected from chloro, fluoro, cyano, methyl, and methoxy; or
B. tetralin-1-yl, tetralin-2-yl, indan-1-yl, or indan-2-yl, or optionally substituted with one or two substituents, for instance one substituent, each independently selected from chloro, fluoro, cyano, methyl, and methoxy; or
C. tetralin-1-yl or indan-1-yl substituted with one substituent selected from chloro, fluoro, cyano, methyl, and methoxy.

In an embodiment of each aspect of the invention, R^{G}, R^{G2}, R^{G3}, and R^{G4} are independently selected from:
A. hydroxyl, halogen, such as chloro and fluoro, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, or cyclopropyloxy; or
B. hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
C. hydroxyl, chloro, fluoro, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl; or
D. chloro, fluoro, cyano, methyl, and methoxy; or
E. chloro, and methyl.

In an embodiment of each aspect of the invention, A is selected from:
A. A-1, A-2, A-3, A-4, A-5, A-6, A-7, A-8, A-9, A-10, A-11, A-12, A-13, A-14, A-15, A-16, and A-17; or
B. A-1, A-2, A-3, A-4, A-5, A-6, A-7, A-8, A-9, A-10, A-11, and A-12; or
C. A-13, A-14, A-15, A-16, and A-17; or
D. A-1, A-2, A-3, A-4, A-5, and A-6; or
E. A-7, A-8, A-9, A-10, A-11, and A-12; or
F. A-13, A-14, A-15, A-16, and A-17; or
G. A-1, A-2, A-3, A-13, A-14, and A-15; or
H. A-4, A-5, A-6, A-7, A-8, A-9, A-15, A-16, and A-17; or
I. A-10, A-11, and A-12; or
J. A-1, A-2, A-3, A-7, A-8, A-9, A-13, A-14, and A-15; or
K. A-4, A-5, A-6, A-10, A-11, A-12, A-16, and A-17; or
L. A-1, A-3, A-5, A-13, and A-15; or
M. A-1, A-3, and A-5; or
N. A-1, A-3 and A-13; or
O. A-1, A-3 and A-15; or
P. A-1 and A-3; or
Q. A-1 and A-5; or
R. A-3 and A-5;
S. A-3.

In an embodiment of each aspect of the invention, A is selected from A-1 to A-17, wherein R⁷, R⁸ and R⁹ are independently selected from:
A. hydrogen, fluoro, chloro, bromo, iodo, methyl, ethyl, propyl, isopropyl, allyl, propargyl, cyclopropylmethyl, cyclopropyl, cyclopentyl, cyclohexyl, —C(═O)OCH₃, —C(═O)N(CH₃)₂, 2-(dimethylamino)-2-oxo-ethyl, 2-(methylamino)-2-oxo-ethyl, difluoromethyl, trifluoromethyl, methylsulfonyl, methylsulfanyl, thiomethoxy, methoxy, ethoxy, cyano, hydroxyl, mercapto, and amino; or
B. hydrogen, fluoro, chloro, methyl, ethyl, isopropyl, cyclopropyl, —C(═O)OCH₃, —C(═O)N(CH₃)₂, 2-(dimethylamino)-2-oxo-ethyl, 2-(methylamino)-2-oxo-ethyl, difluoromethyl, trifluoromethyl, methylsulfonyl, methoxy, and cyano; or
C. hydrogen, fluoro, chloro, methyl, ethyl, cyclopropyl, 2-(methylamino)-2-oxo-ethyl, methylsulfonyl, methoxy, and cyano; or
D. hydrogen, fluoro, chloro, methyl, cyclopropyl, and cyano; or
E. hydrogen, fluoro, chloro, methyl, and cyano; or
F. hydrogen, fluoro, chloro, and methyl; or
G. hydrogen, chloro, and methyl; or
H. hydrogen, chloro, and fluoro.

In an embodiment of each aspect of the invention, A is selected from A-1 to A-17, and at least one of R⁷, R⁸ and R⁹ is hydrogen, and the others, when present, are selected from:
A. hydrogen, fluoro, chloro, methyl, ethyl, cyclopropyl, 2-(methylamino)-2-oxo-ethyl, methylsulfonyl, methoxy, and cyano; or
B. hydrogen, fluoro, chloro, methyl, cyclopropyl, and cyano; or
C. hydrogen, fluoro, chloro, methyl, and cyano; or
D. hydrogen, fluoro, chloro, and methyl; or
E. hydrogen, chloro, and methyl; or
F. hydrogen, chloro, and cyano; or
G. hydrogen, chloro, and fluoro; or
H. hydrogen, and chloro.

Preferred embodiment 1 of each aspect of the invention provides compounds of formula (I-A-1), (I-A-3), (I-A-5), (I-A-13) and (I-A-15):

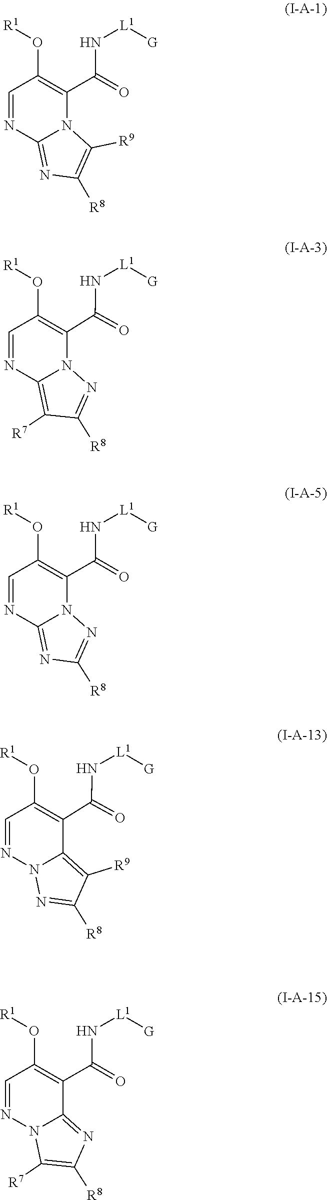

or an agrochemically acceptable salt, stereoisomer, enantiomer, and N-oxide thereof, wherein G, L¹, R¹, R², R³, R⁴, R⁵, R⁷, R⁸, R⁹ are as defined for a compound of formula (I).

Preferred embodiment 2 of each aspect of the invention provides compounds according to preferred embodiment 1, wherein L¹ is a direct bond, or —CR²R³—CR⁴R⁵— wherein R² and R³ are independently selected from hydrogen and methyl; and R⁴ and R⁵ are independently selected from hydrogen, hydroxy, fluoro, methyl, cyano, and methoxy.

Preferred embodiments 3 of each aspect of the invention provides compounds according to preferred embodiments 1 or 2, wherein L¹ is —CR²R³—CR⁴R⁵— wherein R² and R³ are hydrogen; and R⁴ and R⁵ are independently selected from hydrogen, hydroxy, fluoro, methyl, cyano, and methoxy, preferably from hydrogen and fluoro.

Preferred embodiments 4 of each aspect of the invention provides compounds according to any one of preferred embodiments 1, 2 or 3, wherein L¹ is —CH₂—CHF— or —CH₂—CH₂— (i.e. R² and R³ are hydrogen, and R⁴ or R⁵ is fluoro and the other is hydrogen).

Preferred embodiment 5 of each aspect of the invention provides compounds according to any one of preferred embodiments 1, 2, 3 or 4, wherein R¹ is phenyl, pyridyl, or pyrimidyl, each optionally substituted with one or two substituents R¹¹ independently selected from halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl.

Preferred embodiment 6 of each aspect of the invention provides compounds according to any one of preferred embodiments 1, 2, 3, 4, or 5, wherein R¹ is phenyl, or pyridyl, each optionally substituted with one or two substituents R¹¹ independently selected from chloro, fluoro, cyano, methyl, methoxy, difluoromethoxy, and cyclopropyl.

Preferred embodiment 7 of each aspect of the invention provides compounds according to any one of preferred embodiments 1, 2, 3, 4, 5, or 6, wherein R¹ is phenyl optionally 3-substituted with one substituent R¹¹ selected from chloro, fluoro, cyano, methyl, methoxy, difluoromethoxy, and cyclopropyl, preferably selected from chloro, cyano, methyl, and cyclopropyl.

Preferred embodiment 8 of each aspect of the invention provides compounds according to any one of preferred embodiments 1, 2, 3, 4, 5, or 6, wherein R¹ is pyridyl, preferably pyrid-3-yl, optionally substituted with one or two substituents R¹¹ independently selected from chloro, fluoro, cyano, methyl, methoxy, difluoromethoxy, and cyclopropyl.

Preferred embodiment 9 of each aspect of the invention provides compounds according to preferred embodiment 8, wherein R¹ is pyrid-3-yl 5-substituted with one substituent R¹¹ selected from chloro, cyano, methyl, and cyclopropyl; preferably cyano.

Preferred embodiment 10 of each aspect of the invention provides compounds according to any one of preferred embodiments 1, 2, 3, 4, 5, 6, 7, 8, or 9, wherein G is phenyl or phenoxy, wherein said phenyl or phenoxy is optionally substituted with 1, 2 or 3 substituents R^{G1} independently selected from hydroxyl, halogen, cyano, methyl, vinyl, ethynyl, difluoromethyl, trifluoromethyl methoxy, ethoxy, allyloxy, propargyloxy, difluoromethoxy, trifluoromethoxy, cyclopropyl, and cyclobutyl.

Preferred embodiment 11 of each aspect of the invention provides compounds according to any one of preferred embodiments 1, 2, 3, 4, 5, 6, 7, 8, 9 or 10, wherein G is phenyl optionally substituted with one or two substituents R^{G1} independently selected from chloro, fluoro, methyl, and methoxy.

Preferred embodiment 12 of each aspect of the invention provides compounds according to any one of preferred embodiments 1, 2, 3, 4, 5, 6, 7, 8, 9, 10 or 11, wherein G is phenyl substituted with two substituents independently selected from chloro and methyl; preferably G is 2,4-dichlorophenyl.

Preferred embodiment 13 of each aspect of the invention provides compounds according to any one of preferred embodiments 1, 2, 3, 4, 5, 6, 7, 8, 9, 10, 11 or 12, wherein R⁷, R⁸ and R⁹ are independently selected from hydrogen, fluoro, chloro, bromo, iodo, methyl, ethyl, propyl, isopropyl, allyl, propargyl, cyclopropylmethyl, cyclopropyl, cyclopentyl, cyclohexyl, —C(═O)OCH₃, —C(═O)N(CH₃)₂, 2-(dimethylamino)-2-oxo-ethyl, 2-(methylamino)-2-oxo-ethyl, difluoromethyl, trifluoromethyl, methylsulfonyl, methylsulfanyl, thiomethoxy, methoxy, ethoxy, cyano, hydroxyl, mercapto, and amino.

Preferred embodiment 14 of each aspect of the invention provides compounds according to any one of preferred embodiments 1, 2, 3, 4, 5, 6, 7, 8, 9, 10, 11, 12 or 13, wherein R⁷, R⁸ and R⁹ are independently selected from hydrogen, fluoro, chloro, methyl, cyclopropyl, and cyano.

Preferred embodiment 15 of each aspect of the invention provides compounds according to any one of preferred embodiments 1, 2, 3, 4, 5, 6, 7, 8, 9, 10, 11, 12, 13 or 14, wherein at least one of R⁷, R⁸ and R⁹ is hydrogen, and the others, when present, are selected from hydrogen, fluoro, chloro, methyl, ethyl, cyclopropyl, 2-(methylamino)-2-oxo-ethyl, methylsulfonyl, methoxy, and cyano.

Preferred embodiment 16 of each aspect of the invention provides compounds according to any one of preferred embodiments 1, 2, 3, 4, 5, 6, 7, 8, 9, 10, 11, 12, 13, 14 or 15, wherein at least one of R⁷, R⁸ and R⁹ is hydrogen, and the others, when present, are selected from hydrogen, fluoro, chloro, methyl, and cyano; preferably from hydrogen, fluoro, chloro, and methyl.

Preferred embodiment 17 of each aspect of the invention provides compounds of formula (I-A-1), (I-A-3), and (I-A-5) according to any one of preferred embodiments 1, 2, 3, 4, 5, 6, 7, 8, 9, 10, 11, 12, 13, 14, 15 or 16.

Preferred embodiment 18 of each aspect of the invention provides compounds of formula (I-A-1), and (I-A-3) according to any one of preferred embodiments 1, 2, 3, 4, 5, 6, 7, 8, 9, 10, 11, 12, 13, 14, 15, or 16.

Preferred embodiment 19 of each aspect of the invention provides compounds of formula (I-A-1) according to any one of preferred embodiments 1, 2, 3, 4, 5, 6, 7, 8, 9, 10, 11, 12, 13, 14, 15, or 16; wherein R⁸ is selected from hydrogen, fluoro, chloro, methyl, and cyano; preferably from hydrogen, fluoro, chloro, and methyl; and R⁹ is hydrogen.

Preferred embodiment 20 of each aspect of the invention provides compounds of formula (I-A-3) according to any one of preferred embodiments 1, 2, 3, 4, 5, 6, 7, 8, 9, 10, 11, 12, 13, 14, 15, or 16, wherein at least one of R⁷ and R⁸ is hydrogen, and the other is selected from hydrogen, fluoro, chloro, methyl, and cyano.

Preferred embodiment 21 of each aspect of the invention provides compounds according to preferred embodiment 20, wherein R⁷ is selected from hydrogen, chloro and methyl, R⁸ is selected from hydrogen, fluoro, chloro, cyano, and methyl, at least one of R⁷ and R⁸ being hydrogen.

Preferred embodiment 22 of each aspect of the invention provides compounds of formula (I-A-5) according to any one of preferred embodiments 1, 2, 3, 4, 5, 6, 7, 8, 9, 10, 11, 12, 13, 14, 15, or 16, wherein R⁸ is selected from hydrogen, fluoro, chloro, and methyl, preferably R⁸ is selected from hydrogen, and methyl.

Preferred embodiment 23 of each aspect of the invention provides compounds according to any one of preferred embodiments 17, 18, 19, 20, 21, or 22, wherein R¹ is phenyl 3-substituted with one substituent R¹¹ selected from chloro, cyano, methyl, and cyclopropyl.

Preferred embodiment 24 of each aspect of the invention provides compounds according to any one of preferred embodiments 17, 18, 19, 20, 21, 22, or 23, wherein L¹ is —CR²R³—CR⁴R⁵— wherein R² and R³ are hydrogen; and R⁴ and R⁵ are independently selected from hydrogen and fluoro.

Preferred embodiment 25 of each aspect of the invention provides compounds according to any one of preferred embodiments 17, 18, 19, 20, 21, 22, 23, or 24, wherein G is phenyl optionally substituted with one or two substituents R^{G1} independently selected from chloro, fluoro, methyl, and methoxy.

Preferred embodiment 26 of each aspect of the invention provides compounds according to any one of preferred embodiments 17, 18, 19, 20, 21, 22, 23, 24, or 25, wherein G is phenyl 2,4-disubstituted with two substituents R^{G1} independently selected from chloro, fluoro, methyl, and methoxy, preferably selected from chloro, fluoro, and methyl.

In one embodiment, the compound of formula (I) according to the invention is selected from compounds listed in any one of Tables A-1 to A-17.

In another embodiment, the compound of formula (I) according to the invention is selected from compounds as listed in Table T1 (below).

In another embodiment of the invention, the compound of formula (I) is selected from 6-(3-cyclopropylphenoxy)-N-[2-(2,4-dichlorophenyl)-2-fluoro-ethyl]imidazo[1,2-a]pyrimidine-5-carboxamide (P-1.1); 6-(3-cyclopropylphenoxy)-N-[2-(2,4-dichlorophenyl)-2-fluoro-ethyl]-[1,2,4]triazolo[1,5-a]pyrimidine-7-carboxamide (P-1.2); 6-(3-cyclopropylphenoxy)-N-[2-(2,4-dichlorophenyl)-2-fluoro-ethyl]pyrazolo[1,5-a]pyrimidine-7-carboxamide (P-1.3); 7-(3-cyclopropylphenoxy)-N-[2-(2,4-dichlorophenyl)-2-fluoro-ethyl]imidazo[1,2-b]pyridazine-8-carboxamide (P-1.4); 6-(3-cyclopropylphenoxy)-N-[2-(2,4-dichlorophenyl)ethyl]-[1,2,4]triazolo[1,5-a]pyrimidine-7-carboxamide (P-1.5); 6-(3-cyclopropylphenoxy)-N-[2-(2,4-dichlorophenyl)ethyl]imidazo[1,2-a]pyrimidine-5-carboxamide (P-1.6); 6-(3-cyclopropylphenoxy)-N-[2-(2,4-dichlorophenyl)-2-fluoro-ethyl]-2-methyl-imidazo[1,2-a]pyrimidine-5-carboxamide (P-1.7); 2-cyano-6-(3-cyclopropylphenoxy)-N-[2-(2,4-dichlorophenyl)-2-fluoro-ethyl]pyrazolo[1,5-a]pyrimidine-7-carboxamide (P-1.8); 3-chloro-6-(3-cyclopropylphenoxy)-N-[2-(2,4-dichlorophenyl)-2-fluoro-ethyl]pyrazolo[1,5-a]pyrimidine-7-carboxamide (P-1.9); 6-(3-cyclopropylphenoxy)-N-[2-(2,4-dichlorophenyl)-2-fluoro-ethyl]-2-fluoro-pyrazolo[1,5-a]pyrimidine-7-carboxamide (P-1.10); 6-(3-cyclopropylphenoxy)-N-[2-(2,4-dichlorophenyl)-2-fluoro-ethyl]-3-methyl-pyrazolo[1,5-a]pyrimidine-7-carboxamide (P-1.11); 2-chloro-6-(3-cyclopropylphenoxy)-N-[2-(2,4-dichlorophenyl)-2-fluoro-ethyl]pyrazolo[1,5-a]pyrimidine-7-carboxamide (P-1.12); 6-(3-cyclopropylphenoxy)-N-[2-(2,4-dichlorophenyl)-2-fluoro-ethyl]-2-methyl-pyrazolo[1,5-a]pyrimidine-7-carboxamide (P-1.13); 6-(3-cyclopropylphenoxy)-N-[2-(2,4-dichlorophenyl)-2-fluoro-ethyl]-2-methyl-[1,2,4]triazolo[1,5-a]pyrimidine-7-carboxamide (P-1.14); 5-(3-cyclopropylphenoxy)-N-[2-(2,4-dichlorophenyl)-2-fluoro-ethyl]pyrazolo[1,5-b]pyridazine-4-carboxamide (P-1.15); 3-chloro-6-(3-cyclopropylphenoxy)-N-[(2R)-2-(2,4-dichlorophenyl)-2-fluoro-ethyl]pyrazolo[1,5-a]pyrimidine-7-carboxamide (P-1.16); 3-chloro-6-(3-cyclopropylphenoxy)-N-[(2S)-2-(2,4-dichlorophenyl)-2-fluoro-ethyl]pyrazolo[1,5-a]pyrimidine-7-carboxamide (P-1.17); 3-cyano-6-(3-cyclopropylphenoxy)-N-[2-(2,4-dichlorophenyl)-2-fluoro-ethyl]-2-methoxy-pyrazolo[1,5-a]pyrimidine-7-carboxamide (P-1.18); 3-chloro-6-(3-cyclopropylphenoxy)-N-[2-(2,4-dichlorophenyl)-2-fluoro-ethyl]-2-methyl-pyrazolo[1,5-a]pyrimidine-7-carboxamide (P-1.19); or 3-cyano-6-(3-cyclopropylphenoxy)-N-[2-(2,4-dichlorophenyl)-2-fluoro-ethyl]-2-methyl-pyrazolo[1,5-a]pyrimidine-7-carboxamide (P-1.20) according to Table T1 (below).

The presence of one or more possible asymmetric carbon atoms in any of the compounds selected from compounds of formula (I), or compounds selected from compounds listed in Tables A-1 to A-17, or compounds listed in Table T1 (below), according to the invention means that the compounds may occur in chiral isomeric forms, i.e., enantiomeric or diastereomeric forms.

The compounds of formula (I) can be prepared by those skilled in the art as shown in the following schemes 1 to 29, wherein A, G, L¹, R¹, R², R³, R⁴, R⁵, R⁷, R⁸, R⁹ and R¹¹ are as defined for a compound of formula (I), unless otherwise stated. Certain stereogenic centers have been left unspecified for the clarity and are not intended to limit the teaching of the schemes in any way.

As shown in Scheme 1, compounds of formula (I) may be obtained by an amide-coupling transformation with compounds of formula (II) and amine compounds of formula (III) by activating the carboxylic acid function of the compounds of formula (II), a process that usually takes place by converting the —OH of the carboxylic acid into a good leaving group, such as a chloride group, for example by using (COCl)₂ or SOCl₂, prior to treatment with the compounds of formula (III), preferably in a suitable solvent (e.g., N-methylpyrrolidone, acetonitrile, dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile) optionally in the presence of a base (e.g., triethylamine or N,N-diisopropylethylamine). For examples, see Chem. Soc. Rev. 2009, 38, 606 and Chem. Soc. Rev. 2011, 40, 5084. Compounds of formula (III) are either known or commercially available.

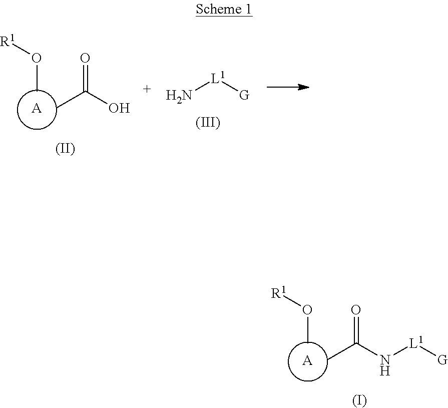

As shown in Scheme 2, compounds of formula (I) may also be prepared by reacting nucleophilic compounds of formula (IV) with electrophilic compounds of formula (V), wherein X² is a suitable leaving group such as fluoro, chloro, bromo, iodo, BF₃K, B(OH)₂ or B(pinacol), in the presence of a base (e.g., KO-t-Bu, K₃PO₄, K₂CO₃, triethylamine, or Cs₂CO₃), in a suitable solvent (e.g., N-methylpyrrolidone, dimethylacetamide, acetonitrile, tetrahydrofuran, 2-methyl tetrahydrofuran, sulfolane, or dimethylsulfoxide) at temperatures between 10° C. and 90° C. and preferably using a metal catalyst complex (e.g., Cu or Pd).

For related examples, see Eur. J. Org. Chem. 2011, 18, 3353; J. Org. Chem. 2009, 74, 7951; Tetrahedron Lett. 2012, 53, 5318. Compounds of formula (IV) are either known or commercially available.

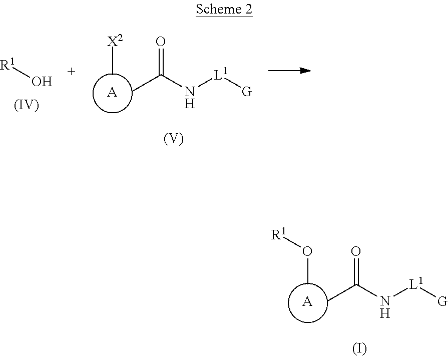

As shown in Scheme 3, compounds of formula (II) may be obtained by hydrolysis of compounds of formula (VII), wherein X¹ is C₁-C₄-alkoxy, e.g., methoxy or ethoxy, using an alkali metal hydroxide (e.g., NaOH, LiOH) in a suitable solvent or mixture of solvents, typically tetrahydrofuran, methanol, water, 2-methyl-tetrahydrofuran, acetonitrile at temperatures between 20° C. and 100° C. For related examples, see WO 2008/133192 or J. Med. Chem. 2021, 64, 12322.

Compounds of formula (VII), wherein X¹ is C₁-C₄-alkoxy, for instance methoxy or ethoxy, may be prepared by reacting nucleophilic compounds of formula (IV) with electrophilic compounds of formula (VI), wherein X¹ is C₁-C₄-alkoxy, such as methoxy or ethoxy, and X² is a suitable leaving group such as fluoro, chloro, bromo, iodo, BF₃K, B(OH)₂ or B(pinacol), in the presence of a base (e.g., KO-t-Bu, K₃PO₄, K₂CO₃, triethylamine, or Cs₂CO₃) in a suitable solvent or mixture of solvents (e.g., N-methylpyrrolidone, dimethylacetamide, acetonitrile, tetrahydrofuran, 2-methyl tetrahydrofuran, sulfolane, dimethylsulfoxide) at temperatures between 20° C. and 110° C. and preferably using a metal catalyst complex (e.g., Cu or Pd). For related examples, see Eur. J. Org. Chem. 2011, 18, 3353; J. Org. Chem. 2009, 74, 7951; Tetrahedron Lett. 2012, 53, 5318; WO 2008/110313 and WO 2012/136604. Compounds of formula (IV) are either known or commercially available.

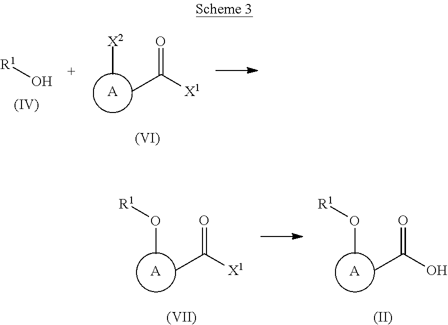

As shown in Scheme 4, compounds of formula (V), wherein X² is a suitable leaving group such as fluoro, chloro, bromo, iodo, BF₃K, B(OH)₂ or B(pinacol), may be obtained by an amide coupling transformation with compounds of formula (VIII), wherein X² is a suitable leaving group such as fluoro, chloro, bromo, iodo, BF₃K, B(OH)₂ or B(pinacol), and amine compounds of formula (III) by activating the carboxylic acid function of the compounds of formula (VIII), a process that usually takes place by converting the —OH of the carboxylic acid into a good leaving group, such as a chloride group, for example by using (COCl)₂ or SOCl₂, prior to treatment with the compounds of formula (III), preferably in a suitable solvent (e.g., N-methylpyrrolidone dimethylacetamide, dichloromethane or tetrahydrofuran), preferably at temperatures between 25° C. and 60° C., and optionally in the presence of a base such as triethylamine or N,N-diisopropylethylamine; or alternatively under conditions described in the literature for an amide coupling such as 1-propanephosphonic acid cyclic anhydride (T3P) in suitable solvent (e.g., acetonitrile), optionally in the presence of a base (e.g., triethylamine or N,N-diisopropylethylamine). For examples, see: Chem. Soc. Rev. 2009, 38, 606 and Chem. Soc. Rev. 2011, 40, 5084. Compounds of formula (III) are either known or commercially available.

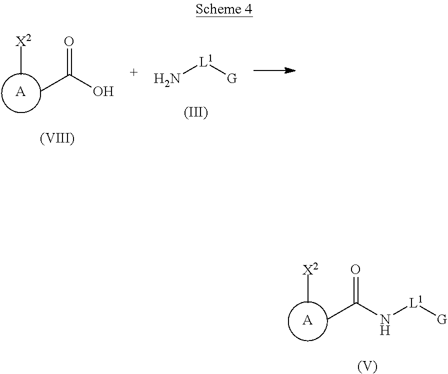

As shown in Scheme 5, compounds of formula (VII), wherein X³ is OH or C₁-C₄-alkoxy, such as methoxy or ethoxy, may also be prepared by reacting nucleophilic compounds of formula (X), wherein X³ is OH or C₁-C₄-alkoxy, such as methoxy or ethoxy, with electrophilic compounds of formula (IX), wherein and X² is a suitable leaving group such as fluoro, chloro, bromo, iodo, BF₃K, B(OH)₂ or B(pinacol), in a suitable solvent (e.g., dichloromethane, 1,2-dichloromethane, acetonitrile, tetrahydrofuran, 2-methyl tetrahydrofuran, N-methylpyrrolidone, dimethylacetamide) at temperatures between 40° C. and 80° C. and using a metal source (e.g., Cu(OAc)₂), and preferably in the presence of an oxidant such as O₂ or a suitable palladium pre-catalyst, such as RockPhos Pd G3, in the presence of a base (e.g., K₃PO₄) and suitable solvent (e.g., dimethyl ether or toluene) at temperatures between 20° C. and 80° C. For related examples, see Org. Lett. 2003, 5, 1381; Tetrahedron Lett. 1998, 39, 2933; Tetrahedron Lett. 2003, 44, 3863 and Org. Lett. 2013, 15, 2876. Compounds of formula (IX) are either known or commercially available.

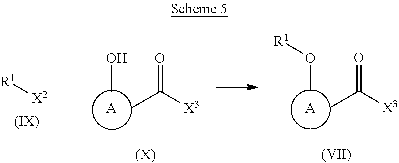

Alternatively, as shown in Scheme 6, compounds of formula (I), may also be prepared by reacting nucleophilic compounds of formula (XI), with electrophilic compounds of formula (IX), wherein and X² is a suitable leaving group such as fluoro, chloro, bromo, iodo, BF₃K, B(OH)₂ or B(pinacol), in a suitable solvent (e.g., dichloromethane, 1,2-dichloromethane, acetonitrile, tetrahydrofuran, 2-methyl tetrahydrofuran) at temperatures between 40° C. and 80° C. and using a metal source (e.g., Cu(OAc)₂), optionally in the presence of an oxidant such as O₂ or a suitable palladium pre-catalyst, such as RockPhos Pd G3, in the presence of a base (e.g., K₃PO₄) and suitable solvent (e.g., dimethyl ether or toluene) at temperatures between 20° C. and 80° C. For related examples, see Org. Lett. 2003, 5, 1381; Tetrahedron Lett. 1998, 39, 2933; Tetrahedron Lett., 2003, 44, 3863 and Org. Lett. 2013, 15, 2876. Compounds of formula (IX) are either known or commercially available.

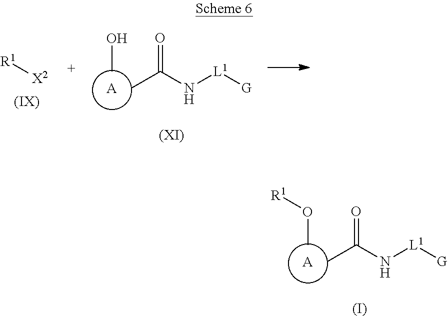

As shown in Scheme 7, compounds of formula (XIV), wherein X⁵ is OH or halogen, may be obtained from compounds of formula (XIII) by hydrolysis in a similar manner as described for the conversion of compounds of formula (VII) into compounds of formula (II) in Scheme 3. Compounds of formula (XIII), wherein X³ is OH or C₁-C₄-alkoxy, such as methoxy or ethoxy, and X⁵ is OH or halogen may be obtained from compounds of formula (XII), wherein X⁵ is OH or halogen, via an oxidation method using a suitable oxidant, such as KMnO₄ or a cobalt(II) salt and trihydroxyisocyanuric acid (THICA) in a suitable solvent (e.g., acetic acid) at temperatures between 25° C. and 200° C. For related examples, see: Can. J. Chem. 1978, 56, 1273 and WO 2021/160470. Compounds of formula (XII) are known or can be prepared as described in Bulletin de la Societe Chimique de France 1972, 8, 3198.

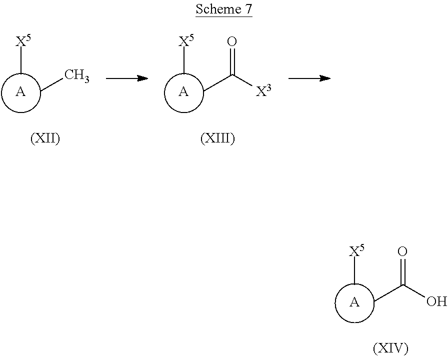

As shown in Scheme 8, compounds of formula (VII), wherein X¹ is C₁-C₄-alkoxy, such as methoxy or ethoxy, may be obtained from compounds of formula (XV) via an oxidation method using a suitable oxidant, such as KMnO₄ or a suitable cobalt(II) salt and trihydroxyisocyanuric acid (THICA) in a suitable solvent (e.g., acetic acid) at temperatures between 25° C. and 200° C. For related examples, see: Can. J. Chem. 1978, 56, 1273 and WO 2021/160470. Compounds of formula (XV) may be prepared as described in Bulletin de la Societe Chimique de France 1972, 8, 3198.

Furthermore, compounds of formula (XV), may be prepared by reacting nucleophilic compounds of formula (IV) with electrophilic compounds of formula (XVI), wherein X² is a suitable leaving group such as fluoro, chloro, bromo, iodo, BF3K, B(OH)₂ or B(pinacol), in the presence of base (e.g., KO-t-Bu, K₃PO₄, K₂CO₃, triethylamine, or Cs₂CO₃), in a suitable solvent (e.g., N-methylpyrrolidone, dimethylacetamide, acetonitrile, tetrahydrofuran, 2-methyl tetrahydrofuran, sulfolane, dimethylsulfoxide) at temperatures between 25° C. and 100° C., and optionally using a metal catalyst and ligand complex (e.g., Cul, N,N-dimethylglycine). For related examples, see Eur. J. Org. Chem. 2011, 18, 3353; J. Org. Chem. 2009, 74, 7951; Tetrahedron Lett. 2012, 53, 5318; WO 2008/110313 and WO 2012/136604. Compounds of formula (IV) are either known or commercially available.

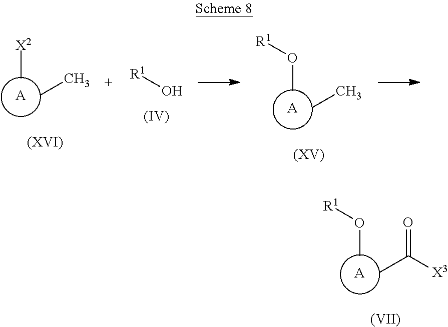

As shown in Scheme 9, compounds of formula (VII), wherein X³ is OH or C₁-C₄-alkoxy may also be prepared by reaction of compounds of formula (XVII) in aqueous solvent mixture such as iso-propanol or ethanol, optionally in an alkaline media at temperatures between 90° C. and 110° C. For related examples, see: J. Med. Chem. 2012, 55, 10118.

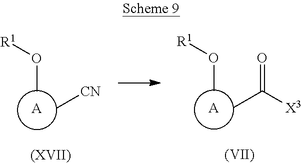

Compounds of formula (XVII) are known or, as shown in Scheme 10. they may be obtained from compounds of formula (XVIII) wherein X⁶ is chloro, bromo, iodo, or trifluoromethanesulfonyl-O—, at temperatures between 50° C. and 120° C., preferably between 80° C. and 110° C., using a metal source, such as XPhos Pd G1 (also called: (2-Dicyclohexylphosphino-2′,4′,6′-triisopropyl-1,1′-biphenyl)[2-(2-aminoethyl)phenyl)]palladium(II) chloride, or (XPhos) palladium(II) phenethylamine chloride, or Chloro(2-dicyclohexylphosphino-2′,4′,6′-triisopropyl-1,1′-biphenyl)[2-(2-aminoethyl)phenyl)]palladium(II), or XPhos Palladacycle, or XPhos precatalyst), in the presence of a cyanide source, such as potassium ferrocyanide, cooper cyanide, zinc cyanide or potassium cyanide, and a base (e.g., KOAc) in a suitable solvent or a mixture of solvents (e.g., dioxane, water, toluene, tetrahydrofuran, 2-methyl-tetrahydrofuran, xylene). For related examples, see: J. Org. Chem. 2018, 83, 4922 and Org. Lett. 2006, 8, 1189.

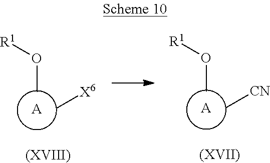

As shown in Scheme 11a, compounds of formula (VII), wherein X³ is OH or C₁-C₄-alkoxy may also be obtained from compounds of formula (XVIII) where X⁶ is chloro, bromo, iodo, or trifluoromethanesulfonyl-O—, at temperatures between 20° C. and 130° C., preferably between 70° C. and 110° C., using a metal source such as XPhos Pd G1 in a pressure vessel, typically a stainless steel autoclave, loaded with carbon monoxide, at a pressure typically between 1 to 50 bar, more preferably between 5 to 15 bar, in the presence of an organic base, for instance triethylamine or diisopropylethylamine, and an appropriate solvent (e.g., methanol or ethanol). For related examples, see: J. Med. Chem. 2014, 57, 2692 and Adv. Synth. Catal. 2006, 348, 1255.

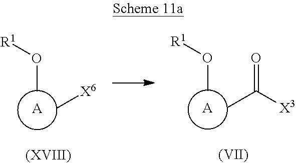

Alternatively, as shown in Scheme 11b, compounds of formula (II) may also be obtained by reacting compounds of formula (XIX) where X⁷ is chloro, bromo or iodo, with a lithium reagent (e.g., n-butyl lithium, sec-butyl lithium, tert-butyl lithium or lithium diisopropylamine) at temperatures between −78° C. and −30° C. in an appropriate solvent, for instance hexane, diethyl ether or tetrahydrofuran, followed by the addition of carbon dioxide. For related examples, see: J. Am. Chem. Soc. 2018, 140, 9140 and J. Am. Chem. Soc. 2021, 143, 1539.

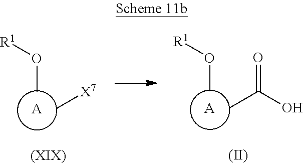

As shown in Scheme 12, compounds of formula (XIX), wherein X⁷ is chloro, bromo or iodo, are either known or may be prepared by reacting compounds of formula (XX) with an electrophilic halogen reagent like bromine, dibromohydantoin, N-bromo- or N-chloro-succinimide, at temperatures between −78° C. and 10° C., optionally with a base, typically lithium diisopropylamine or n-butyl lithium, in a suitable solvent (e.g., chloroform, methyltetrahydrofuran or dimethylformamide). For related examples, see: Tetrahedron Lett. 2003, 44, 823; J. Am. Chem. Soc. 2010, 132, 8858 and Synthesis 2005, 16, 2782.

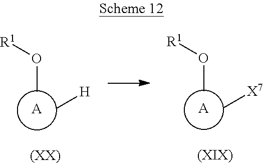

As shown in Scheme 13, compounds of formula (II) may also be prepared by reacting compounds of formula (XX) at temperature between −78° C. and 10° C. with a base, typically lithium diisopropylamine or n-butyl lithium, optionally in the presence of a catalyst (e.g., potassium tert-butoxide), in a suitable solvent (e.g., diethyl ether, cyclopentyl methyl ether, methyl tert-butyl ether or tetrahyrofuran) followed by the addition of carbon dioxide. Compounds of formula (XX) are either known or commercially available. For related examples, see: Bioorg. Med. Chem. 2004, 12, 5579; J. Am. Chem. Soc. 2010, 132, 8858 and Synthesis 2005, 16, 2782.

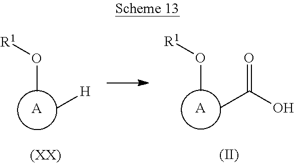

As shown in Scheme 14, compounds of formula (XIX), wherein X⁷ is chloro, bromo or iodo are either known or may be obtained from the tautomeric compounds of formulae (XXI) and (XXII), by treatment with phosphorous compounds, for instance phophorous oxychloride, phosphorous pentachloride or phosphorous tribromide in a suitable solvent (e.g., toluene, acetonitrile, dichloromethane or chloroform) optionally in the presence of a substoichiometric amount of a catalyst (e.g., triphenylphosphine or 4-(dimethylamino)pyridine). For related examples, see: Tetrahedron Lett. 2012, 53, 674; Synth. Commun. 2016, 46, 1619 and J. Org. Chem. 2011, 76, 4149.

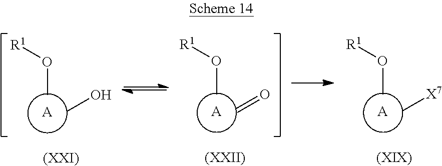

As shown in Scheme 15, compounds of formula (XXI) in equilibrium with compounds of formula (XXII) are either known or may be prepared by one-pot nucleophilic addition followed by cyclization using compounds of formula (XXIII), wherein X⁸ are C₁-C₄alkyl or C₅-C₆cycloalkyl, or form together a saturated heterocycle with the nitrogen they are attached to, and X³ is OH or C₁-C₄-alkoxy, and compounds of formula (XXIV), wherein X⁹ are independently carbon atoms substituted by R⁷, R⁸ or R⁹ as defined for the compound of formula (I), or nitrogen atoms, and X¹⁰ is H or a protective group (e.g., tetrahydropyran, 2-(trimethylsilyl)ethoxymethyl or benzyl), preferably in a suitable solvent, for instance ethanol, isopropanol, dimethylformamide, acetic acid or acetonitrile at temperatures between 50° C. and 110° C., optionally in the presence of a base (e.g., potassium carbonate, triethylamine). For related examples, see: US 2018/0230157; Synthesis 2006, 1, 59 and WO 2010/016005. Compounds of formula (XXIV) are either known or commercially available.

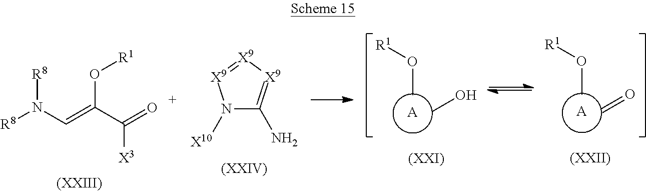

As shown in Scheme 16, compounds of formula (XXI) in equilibrium with compounds of formula (XXII) can be prepared by intramolecular cyclisation of compounds of formula (XXV), wherein X⁸ are C₁-C₄alkyl or C₅-C₆cycloalkyl, or form both a saturated heterocycle with the nitrogen they are attached to, and X⁹ are independently carbon atoms substituted by R⁷, R⁸ or R⁹ as defined for the compound of formula (I), or nitrogen atoms, in a suitable solvent, for instance water or ethanol at temperatures between 20° C. and 100° C. For related examples, see: WO 2005/012262.

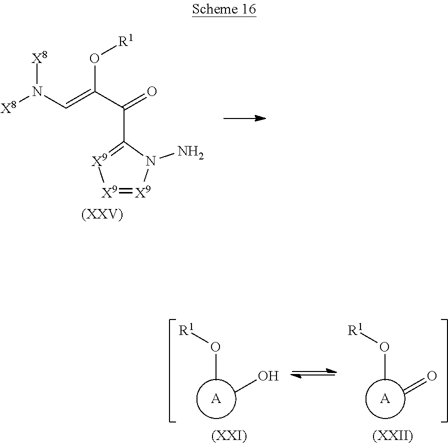

As shown in Scheme 17, compounds of formula (XXI) are in equilibrium with compounds of formula (XXII), and can be prepared by amination and direct cyclization of compounds of formula (XXVI), wherein X⁸ are C₁-C₄alkyl or C₅-C₆cycloalkyl, or form both a saturated heterocycle with the nitrogen they are attached to, and X⁹ are independently carbon atoms substituted by R⁷, R⁸ or R⁹ as defined for the compound of formula (I), or nitrogen atoms, and X¹⁰ is H or a protective group (e.g., tetrahydropyran, 2-(trimethylsilyl)ethoxymethyl or benzyl) by reaction with an amination reagent (e.g., amino 4-nitrobenzoate, N-(tert-Butoxycarbonyl)-2-nitrobenzenesulfonamide, hydroxylamine-O-sulfonic acid or sodium diformylamide) in a suitable solvent, for instance dimethylsulfoxide, dimethylformamide, dichloromethane, N-Methyl-2-pyrrolidone or ethanol at temperatures between 0° C. and 110° C., more preferably between 20° C. and 50° C., optionally in the presence of a base (e.g., KOH, NaOH). For related examples, see: J. Med. Chem. 1996, 39, 582; Organometallics 2014, 33, 4035 and Chem. Eur. J. 2019, 25, 1963.

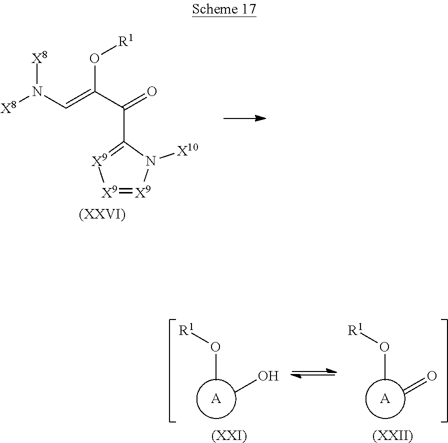

As shown in Scheme 18, compounds of formula (XXV), wherein X⁸ are C₁-C₄alkyl or C₅-C₆cycloalkyl, or form both a saturated heterocycle with the nitrogen they are attached to, and X⁹ are independently carbon atoms substituted by R⁷, R⁸ or R⁹ as defined for the compound of formula (I), or nitrogen atoms, can be obtained by amination of compounds of formula (XXVII), wherein X⁸ are C₁-C₄alkyl or C₅-C₆cycloalkyl, or form both a saturated heterocycle with the nitrogen they are attached to, and X⁹ are independently carbon atoms substituted by R⁷, R⁸ or R⁹ as defined for the compound of formula (I), or nitrogen atoms, in a suitable solvent, for instance, dimethylsulfoxide, N-methyl-2-pyrrolidone or dimethylacetamide, at temperatures between 20° C. and 120° C. For related examples, see: Dalton Trans. 2016, 45, 15644, J. Med. Chem. 1996, 39, 582 or Chem. Eur. J. 2019, 25, 1963.

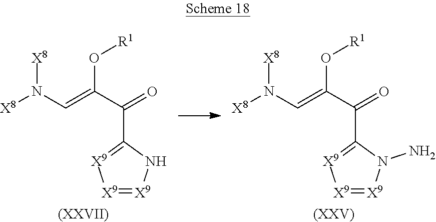

As shown in Scheme 19, compounds of formula (XXVI), wherein X⁸ are C₁-C₄alkyl or C₅-C₆cycloalkyl, or form both a saturated heterocycle with the nitrogen they are attached to, X⁹ are independently carbon atoms substituted by R⁷, R⁸ or R⁹ as defined for the compound of formula (I), or nitrogen atoms, and X¹⁰ is H or a protective group (e.g., tetrahydropyran, 2-(trimethylsilyl)ethoxymethyl or benzyl) can be obtained from compounds of formula (XVVIII), wherein X⁹ are independently carbon atoms substituted by R⁷, R⁸ or R⁹ as defined for the compound of formula (I), or nitrogen atoms, and X¹⁰ is H or a protective group (e.g., tetrahydropyran, 2-(trimethylsilyl)ethoxymethyl or benzyl) by reacting with a formamide equivalent, for instance dimethylformamide dimethyl acetal or 1-(dimethoxymethyl)pyrrolidine, neat or in a suitable solvent (e.g., toluene, dimethylformamide), at temperatures between 50° C. and 120° C. For related examples, see: WO 2021/2296621; WO 2008/149379 and Bioorg. Med. Chem. Lett. 2022, 61, 128552.

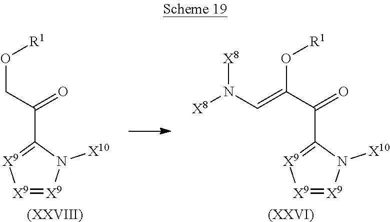

As shown in Scheme 20, compounds of formula (XXVI), wherein X⁸ are C₁-C₄alkyl or C₅-C₆cycloalkyl, or form both a saturated heterocycle with the nitrogen they are attached to, X⁹ are independently carbon atoms substituted by R⁷, R⁸ or R⁹ as defined for the compound of formula (I), or nitrogen atoms, and X¹⁰ is H or a protective group (e.g., tetrahydropyran, 2-(trimethylsilyl)ethoxymethyl or benzyl) can be prepared from compounds of formula (XXIX) and compounds of formula (XXX), wherein X⁷ is chloro, bromo or iodo, X⁹ are independently carbon atoms substituted by R⁷, R⁸ or R⁹ as defined for the compound of formula (I), or nitrogen atoms, and X¹⁰ is H or a protective group (e.g., tetrahydropyran, 2-(trimethylsilyl)ethoxymethyl or benzyl) using an organometallic reagent (e.g., methyl magnesium bromide, isopropylmagnesium chloride lithium chloride, butyl lithium, tert-butyl lithium or phenyl magnesium chloride) at temperatures between −78° C. and 15° C., preferably between −20° C. and 10° C. in a suitable solvent (e.g., tetrahydrofuran, diethyl ether, cyclopentyl methyl ether or 2-methyl tetrahydrofuran). For related examples, see: WO 2009/130193 and WO 2015/057205.

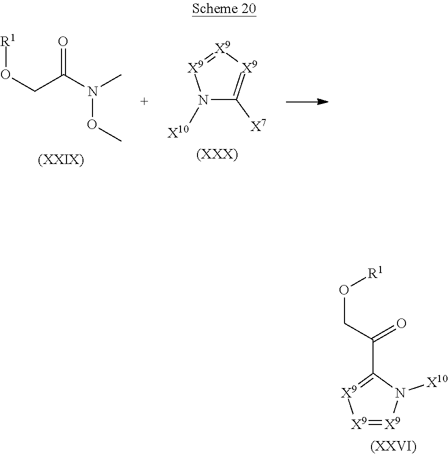

As shown in Scheme 21, compounds of formula (XXIX) are either known or can be obtained by reacting compounds of formula (XXXI), with N,O-dimethylhydroxylamine hydrochloride in the presence of not of a mixture of oxalyl chloride or thionyl chloride and dimethylformamide in a suitable solvent or a mixture of solvents (e.g., dimethylformamide, dichloromethane or 2-methyl tetrahydrofuran) at temperatures between −10° C. and 80° C., more preferably between 0° C. and 30° C. or in presence of a coupling agent (e.g., T3P, HATU, COMU) in a suitable solvent or mixture of solvents, for instance dimethylformamide, dichloromethane, trichloromethane, tetrahydrofuran, methyltetrahydrofuran at temperatures between 0° C. and 80° C. For related examples, see: WO 2011/023706 and J. Med. Chem. 2004, 47, 2405. Compounds of formula (XXXI) are either known or commercially available.

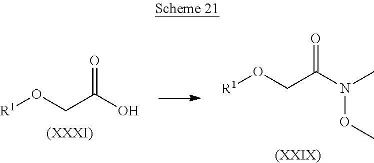

As shown in Scheme 22, compounds of formula (XXIII), wherein X³ is OH or C₁-C₄-alkoxy, X⁸ are C₁-C₄alkyl or C₅-C₆cycloalkyl, or form both a saturated heterocycle with the nitrogen they are attached to, may be prepared by reacting compounds of formula (XXXII), wherein X³ is OH or C₁-C₄-alkoxy in the presence of C₁-C₆-alkoxy- or C₁-C₆-cycloalkoxy-N,N,N′,N′tetra(C₁-C₆-alkyl or C₁-C₆-cycloalkyl)methanediamine (e.g., methoxy- or tert-butoxy-N,N,N′,N′tetramethylmethanediamine) neat or in a suitable solvent or mixture of solvents (e.g., dimethylformamide, 2-methyl tetrahydrofuran) at temperatures between 25° C. and 120° C. For related examples, see: Tetrahedron 1998, 54, 9799; J. Het. Chem. 2014, 51, 954 and Synth. Commun. 2021, 51, 2160. Compounds of formula (XXXII) are either known or commercially available.

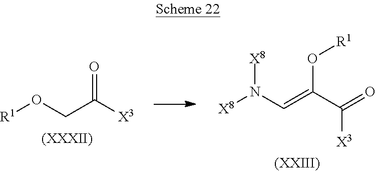

As shown in Scheme 23a, compounds of formula (XXI) are in equilibrium with compounds of formula (XXII), and may be prepared from reacting nucleophilic compounds of formula (XXXIII) in equilibrium with compounds of formula (XXXIV), with electrophilic compounds of formula (IX), wherein X² is a suitable leaving group such as fluoro, chloro, bromo, iodo, BF₃K, B(OH)₂ or B(pinacol), in a suitable solvent (e.g., dichloromethane, 1,2-dichloromethane, acetonitrile, tetrahydrofuran, 2-methyl tetrahydrofuran, N-methylpyrrolidone, dimethylacetamide) at temperatures between 40° C. and 80° C. and using a catalyst (e.g., Cu(OAc)₂), and preferably in the presence of an oxidant such as O₂; alternatively using a suitable palladium pre-catalyst, such as RockPhos Pd G3, in the presence of a base (e.g., K₃PO₄) and suitable solvent (e.g., dimethyl ether or toluene) at temperatures between 20° C. and 80° C. For related examples, see Org. Lett. 2003, 5, 1381; Tetrahedron Lett. 1998, 39, 2933; Tetrahedron Lett. 2003, 44, 3863 and Org. Lett. 2013, 15, 2876.

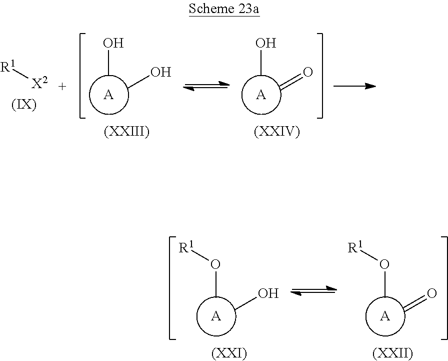

Alternatively, as shown in Scheme 23b, compounds of formula (XXI) in equilibrium with compounds of formula (XXII), may be prepared from compounds of formula (XXXV) in equilibrium with compounds of formula (XXXVI), wherein X⁷ is chloro, bromo or iodo by reaction with compounds of formula (IX), optionally in the presence of a base (e.g. KO-t-Bu, K₃PO₄, K₂CO₃, triethylamine, or Cs₂CO₃), in a suitable solvent or mixture of solvents (e.g., N-methylpyrrolidone, dimethylacetamide, acetonitrile, tetrahydrofuran, 2-methyl tetrahydrofuran, sulfolane, dimethylsulfoxide) at temperatures between 50° C. and 110° C. and preferably using a metal catalyst complex (e.g., Cu or Pd). For related examples, see Eur. J. Org. Chem. 2011, 18, 3353; J. Org. Chem. 2009, 74, 7951; Tetrahedron Lett. 2012, 53, 5318; WO 2008/110313 and WO 2012/136604. Compounds of formula (IX) are either known or commercially available.

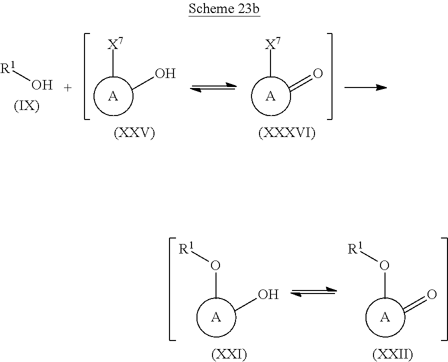

As shown in Scheme 24, compounds of formula (XXXV) are in equilibrium with compounds of formula (XXXVI), and may be obtained from compounds of formula (XXXVII) in equilibrium with compounds of formula (XXXVIII), wherein X⁷ is chloro, bromo or iodo, after treatment with hydrochloric acid or hydrogen bromide or in the optional presence of transition metal (e.g., zinc, palladium) or sodium nitrite followed by the addition of a copper salt, for instance bromide or chloride in a suitable solvent or mixture of solvents (e.g., water, acetonitrile, ethanol) at temperatures between 0° C. and 50° C. For a related example, see: Mendeleev Commun. 2017, 27, 285.

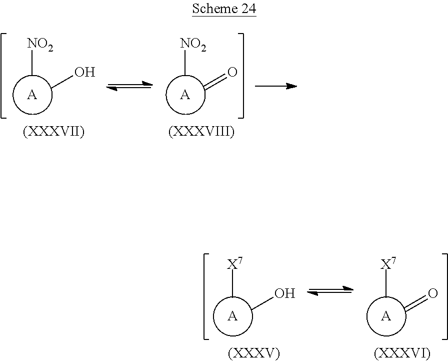

As shown in Scheme 25, compounds of formula (XXXVII) are in equilibrium with compounds of formula (XXXVIII), and may be obtained from compounds of formula (XXXIX), wherein X⁹ are independently carbon atoms substituted by R⁷, R⁸ or R⁹ as defined for the compound of formula (I), or nitrogen atoms, X¹⁰ is H or a protective group (e.g., tetrahydropyran, 2-(trimethylsilyl)ethoxymethyl or benzyl) after treatment with sodium or potassium nitrite, sodium hypochloride or sodium azide in the presence of a strong acide (e.g., hydrochloric acid, trifluoroacetic acid, sulfuric acid, nitric acid) in a suitable solvent, typically water or acetonitrile at temperatures between −10° C. and 30° C. For related examples, see: J. Org. Chem. 2010, 75, 8487; Bioorg. Med. Chem. Lett. 2020, 30, 127216 and J. Org. Chem. 1987, 52, 5538. The resulting diazonium salt was reacted with a nitro compounds (e.g., 4-(2-nitroethenyl)morpholine, 2-nitroacetaldehyde, 2-Nitropropanedial, 1,3-Diethyl 2-nitropropanedioate) in a suitable solvent or a mixture of solvents, for instance, water or ethanol, at temperatures between 0° C. and 60° C., optionally in the presence of a base (e.g., Na₂CO₃, K₂CO₃, KOH), or alternatively optionally in the presence of a strong acid (e.g., nitric acid, hydrochloric acid). For related examples, see: Mendeleev Commun. 2017, 27, 285; WO 2017/144708 or Khimiya Geterotsiklicheskikh Soedinenii 1986, 5, 662. Compounds of formula (XXXIX) are either known or commercially available.

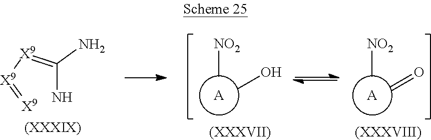

As shown in Scheme 26, compounds of formula (VII), wherein X³ is OH or C₁-C₄-alkoxy, may be obtained by reacting compounds of formula (XL), wherein X³ is OH or C₁-C₄-alkoxy with compounds of formula (XLI), wherein X¹¹ is a leaving group, for instance chloro, bromo, iodo, 0-mesyl, O-tosyl and X¹² a carbonyl or a carbonyl equivalent, for instance an aldehyde, an acetal, an acyl chloride or bromide, an ester or an orthoester, at temperatures between 30° C. and 125° C., optionally in a microwave reactor in a suitable solvent or a mixture of solvents (e.g., dimethylformamide, dimethylsufloxide, toluene, ethanol, iso-propanol, water) and optionally in the presence of a catalyst, for instance hydrogen bromide or sodium carbonate. For related examples, see: J. Med. Chem. 2021, 64, 1197; Tetrahedron Lett. 2017, 58, 4816 and Chem. Pharm. Bull. 1992, 40, 1170. Compounds of formula (XLI) are either known or commercially available.

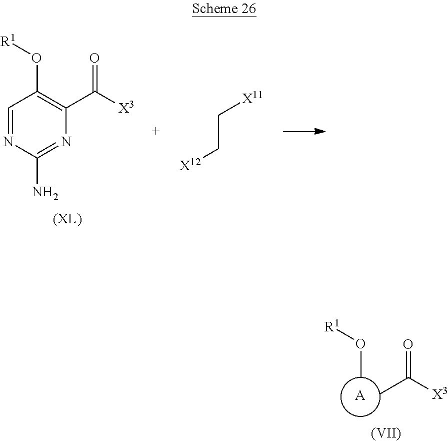

As shown in Scheme 27, compounds of formula (XL), wherein R¹ is as defined for compounds of formula (I), and X³ is OH or C₁-C₄-alkoxy, may be obtained by nucleophilic displacement of sulfones of compounds of formula (XLII), wherein OH or C₁-C₄-alkoxy can be obtained by and X¹³ is an alkyl or cycloalkyl group, for instance methyl, ethyl or cyclohexyl in the presence of an aminating reagent (e.g., NH₄OH, NH₃, NH₄OAc) and the optional presence of a base, for instance triethylamine in a suitable solvent or mixture of solvents (e.g., dimethyl sulfoxide, water, tetrahydrofuran, 1,4-dioxane, iso-propanol, ethanol, methanol, dichloromethane). For related examples, see: Tetrahedron 2009, 65, 1697; Heterocycles 1977, 8, 299 and Org. Biomol. Chem. 2015, 13, 10620

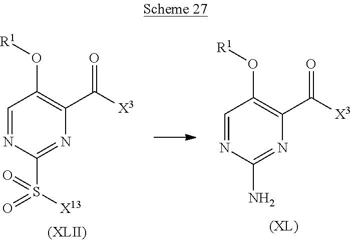

As shown in Scheme 28, compounds of formula (XLII), wherein R¹ is as defined for compounds of formula (I), X³ is OH or C₁-C₄-alkoxy and X¹³ is an alkyl or cycloalkyl group, for instance methyl, ethyl or cyclohexyl, may be obtained by oxidation of compounds of formula (XLIII), wherein R¹, X³ and X¹³ are as defined for compounds of formula (XLII), in the presence of an oxidant (e.g., 3-chloroperoxybenzoic acid, hydrogen peroxide, oxone, chlorine), in a suitable solvent or mixture of solvents, for instance dichloromethane, acetonitrile, chloroform, water, toluene or 2-methyl-tetrahydrofuran, at temperatures between −10° C. and 50° C. For related examples, see: Chem. Eur. J. 2021, 27, 14826; J. Org. Chem. 2017, 82, 2664 and J. Chem. Soc. (C) Organic 1967, 7, 568.

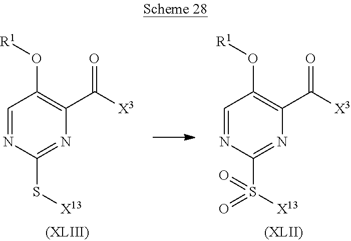

As shown in Scheme 29, compounds of formula (XLIII), wherein R¹ is as defined for compounds of formula (I), X³ is OH or C₁-C₄-alkoxy, for instance methoxy, and X¹³ is an alkyl or cycloalkyl group, for instance methyl, ethyl or cyclohexyl, may be obtained by reacting compounds of formula (XLIV), wherein X³ and X¹³ are as defined for compounds of formula (XLIV), and X¹⁵ is a suitable leaving group such as halogen, in the presence of base (e.g., KO-t-Bu, K₃PO₄, K₂CO₃, triethylamine, or Cs₂CO₃), in a suitable solvent (e.g., N-methylpyrrolidone, dimethylacetamide, acetonitrile, tetrahydrofuran, 2-methyl tetrahydrofuran, sulfolane, dimethylsulfoxide) at temperatures between 25° C. and 110° C., and under reflux and optionally using a metal catalyst and ligand complex (e.g., Cul, N,N-dimethylglycine). For related examples, see Eur. J. Org. Chem. 2011, 18, 3353; J. Org. Chem. 2009, 74, 7951; Tetrahedron Lett. 2012, 53, 5318; WO 2008/110313 and WO 2012/136604. Compounds of formula (XLIV) are either known or commercially available.

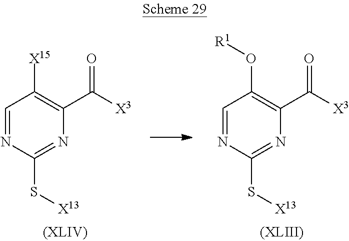

As already indicated, surprisingly, it has now been found that the compounds of formula (I) of the present invention have, for practical purposes, a very advantageous level of biological activity for protecting plants against diseases that are caused by fungi.

The compounds of formula (I) can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) is applied to the plants, to parts thereof or the locus thereof.

It is also possible to use compounds of formula (I) as a fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) can be used for controlling fungi in related areas, for example in the protection of technical materials, including wood and wood related technical products, in food storage, in hygiene management.

In addition, the invention could be used to protect non-living materials from fungal attack, e.g., lumber, wall boards and paint.

The compounds of formula (I), and compositions containing such compounds, are for example, effective in controlling a broad spectrum of plant diseases, such as foliar and/or soil-borne pathogens of ornamental, turf, vegetable, field, cereal, and fruit crops, including fungi and fungal vectors of disease as well as phytopathogenic bacteria and viruses. These pathogens may include:
Oomycetes, including Phytophthora species such as Phytophthora cactorum, Phytophthora capsici, Phytophthora cinnamomi, Phytophthora citricola, Phytophthora citrophthora, Phytophthora erythroseptica, Phytophthora fragariae, Phytophthora infestans, Phytophthora nicotianae, Phytophthora porri, and Phytophthora sojae; Pythium species such as Pythium aphanidermatum, Pythium arrhenomanes, Pythium graminicola, Pythium irregulare and Pythium ultimum; other Peronosporales such as Bremia lactucae, Hyaloperonospora parasitica, Sclerophthora macrospora, Sclerospora graminicola; Peronospora species including Peronospora destructor, Peronospora farinosa f. sp. spinaciae and Peronospora viciae f. sp. pisi; Plasmopara species including Plasmopara halstedii and Plasmopara viticola; Pseudoperonospora species including Pseudoperonospora cubensis and Pseudoperonospora humili; Peronosclerospora species including Peronosclerospora maydis, Peronosclerospora philippinensis and Peronosclerospora sorghi; Albuginales such as Albugo candida, Albugo occidentalis, and Albugo tragopogonis; and Saprolegniales such as Aphanomyces species, including Aphanomyces cochliodes;
Ascomycetes, including Mycosphaerellales such as Actinothyrium graminis, Asperisporium caricae, Cercospora species including Cercospora arachidicola, Cercospora beticola, Cercospora brassicicola, Cercospora canescens, Cercospora cf. flagellaris, Cercospora janseana, Cercospora kikuchii, Cercospora lagenariae, Cercospora sojinae, Cercospora sorghi, Cercospora zeae-maydis; Dothistroma septosporum, Fulvia fulva, Mycosphaerella species including Mycosphaerella pomi and Mycosphaerella linicola; Neopseudocercosporella brassicae, Neopseudocercosporella capsellae, Nothopassalora personata, Nothophaeocrytopus gaeumannii, Passalora bataticola, Passalora koepkei, Pseudocercospora griseola, Pseudocercospora musaei, Pseudocercospora vitis, Pseudocercospora fijiensis, Ramularia species including Ramularia beticola and Ramularia collo-cygni; Ramulariopsis gossypii, Ramulariopsis pseudoglycines, Ramulispora sorghi, Scolecostigmina palmivora, Septoria species including Septoria apiicola, Septoria glycines and Septoria lycopersici; Zasmidium citri-griseum, and Zymosepotria tritici; Helotiales such as Blumeriella jaapii, Botrytis species including Botrytis cinerea and Botrytis fabae; Cadophora gregata, Civorinia allii, Claireedia homoeocarpa, Diplocarpon coronariae, Diplocarpon rosae, Drepanopeziza campestris, Gloeotinia temulenta, Hymenoscyphus fraxineus, Leptotrochila medicaginis, Marssonina graminicola, Monilinia species including Monilinia fructicola, Monilinia fructigena and Monilinia laxa; Neofabraea perennans, Neofabraea vagabunda, Oculimacula yallundae, Pezicula spp., Pseudopeziza medicaginis, Pseudopeziza tracheiphila, Pyrenopeziza species including Pyrenopeziza brassicae; Rhabdocline pseudotsugae, Rhynchosporium species including Rhynchosporium secalis; Sclerotinia species including Sclerotinia minor, Sclerotinia borealis and Sclerotinia sclerotiorum; Hypocreales such as Acremonium strictum, Albifimbria verrucaria, Claviceps africana, Claviceps purpurea, Fusarium species including Fusarium avenaceum, Fusarium culmorum, Fusarium fujikuroi, Fusarium graminearum, Fusarium incarnatum, Fusarium langsethiae, Fusarium moniliforme, Fusarium oxysporum, Fusarium oxysporum f.sp. cubense, Fusarium poae, Fusarium proliferatum, Fusarium pseudograminearum, Fusarium subglutinans, Fusarium sulphureum, Fusarium tricinctum and Fusarium verticillioides; Gliocladium spp., Neocosmospora phaseoli, Neocosmospora solani, Neonectria candida, Paramyrothecium roridum, Sarocladium oryzae, Trichoderma spp. Including Trichoderma harzianum, Trichoderma pseudokoningii and Trichoderma viride; Trichothecium roseum and Ustilaginoidea virens; Magnaporthales such as Gaeumannomyces avenae, Gaeumannomyces graminis, Gaeumannomyces graminis tritici, Gaeumannomyces wongoonoo, Magnaporthiopsis poae, Pyricularia species including Pyricularia grisea and Pyricularia oryzae; Pleosporales, such as Alternaria species including Alternaria allii, Alternaria alternata, Alternaria arachidis, Alternaria brassicae, Alternaria brassicicola, Alternaria dauci, Alternaria grandisi, Alternaria helianthicola, Alternaria linariae, Alternaria mali, Alternaria porri, Alternaria solani and Alternaria tomato; Boeremia coffeae, Ascochyta species including Ascochyta pisi and Ascochyta rabiei; Bipolaris maydis, Bipolaris oryzae, Bipolaris sorokiniana, Cochliobolus spp., Corynespora cassiicola, Curvularia species including Curvularia australiensis, Curvularia cactivora and Curvularia lunata; Didymella species including Didymella pinodella and Didymela pinodes; Drechslera species including Drechslera glycines; Epicoccum nigrum, Exserohilum turcicum, Helminthosporium species including Helminthosporium solani; Hendersonia creberrima, Leptosphaerulina crassiasca, Neocamarosporium betae, Ophiosphaerella agrostidis, Ophiosphaerella herpotricha, Ophiosphaerella korrae, Ophiosphaerella narmari, Parastagonospora nodorum, Phaeosphaeria herpotrichoides, Phaerosphaeria maydis, Phoma spp., Plenodomus lindquistii, Plenodomus lingam, Pleospora spp., Pseudopyrenochaeta lycopersici, Pyrenophora species including Pyrenophora poae, Pyrenophora teres and Pyrenophora tritici-repentis; Remotididymella destructiva, Stagonospora tainanensis, Stagonosporopsis cucurbitacearum, Stemphylium species including Stemphylium botryosum, Stemphylium solani and Stemphylium vesicarium; Diaporthales such as Anisogramma anonmala, Apiognomonia errabunda, Cytospora platani, Diaporthe species including Diaporthe amygdali, Diaporthe helianthin, Diaporthe neoviticola and Diaporthe phaseolorum; Dicarpella spp., Discula destructiva, Gnomoniopsis fructicola, Greeneria uvicola, Juglanconis juglandina, Ophiognomonia clavigignenti-juglandacearum, Stenocarpella maydis and Tubakia dryina; Dothideales such as Aureobasidium species including Aureobasidium pullulans; Discosphaerina fulvida; Erysiphales such as Blumeria graminis, Brasiliomyces malachrae, Erysiphe species including Erysiphe betae, Erysiphe cruciferarum, Erysiphe diffusa, Erysiphe heraclei, Erysiphe necator and Erysiphe pisi; Golovinomyces cichoracearum, Golovinomyces orontii, Leveillula taurica, Oidium arachidisi, Phyllactinia guttata, Podosphaera species including Podosphaera fuliginea, Podosphaera fusca, Podosphaera leucotricha, Podosphaera macularis, Podosphaera mors-uvae, Podosphaera pannosa, Podosphaera tridactyla and Podosphaera xanthii; Glomerellales such as Colletotrichum species including Colletotrichum acutatum, Colletotrichum cereale, Colletotrichum chrysanthemi, Colletotrichum cliviicola, Colletotrichum coccodes, Colletotrichum fragariae, Colletotrichum gloeosporioides, Colletotrichum graminicola, Colletotrichum lentis, Colletotrichum lindemuthianum, Colletotrichum musae, Colletotrichum orbiculare, Colletotrichum siamense and Colletotrichum truncatum; Glomerella cingluata, Glomerella gossypii, Musicillium theobromae, Plectosphaerella cucumerina, Verticillium species including Verticillium dahlia; Venturiales such as Venturia species including Venturia carpophila, Venturia effusa, Venturia inaequalis, Venturia oleaginea, and Venturia pyrina; Xylariales such as Eutypa lata, Microdochium albescens, Microdochium majus, Microdochium nivale, Microdochium paspali, Microdochium sorghi, Physalospora abdita, Rosellinia spp. and Seimatosporium mariae; Botryosphaeriales such as Botryosphaeria species including Botryosphaeria dothidea; Diplodia seriata, Dothiorella aromatica, Lasiodiplodia theobromae, Macrophoma theicola, Macrophomina phaseolina, Phyllosticta ampelicida and Phyllosticta cucurbitacearum; Eurotiales such as Aspergillus species including Aspergillus flavus, Aspergillus fumigatus, Aspergillus niger and Aspergillus terreus; Penicillium species including Penicillium digitatum, Penicillium expansum and Penicillium italicum; Microascales such as Berkeleyomyces basicola, Thielaviopsis paradoxa, Ceratocystis species including Ceratocystis fimbriata, Ceratocystis manginecans and Ceratocystis platani; Scedosporium species including Scedosporium apiospermum and Scedosporium prolificans; Myriangiales such as Elsinoe species including Elsinoe ampelina and Elsinoe perseae; Ophiostomatales such as Leptographium lundbergii, Leptographium microsporum, Ophiostoma novo-ulmi, Ophiostoma piceae and Sporothrix spp.; Pezizomycetes such as Phymatotrichopsis omnivore and Polyscytalum pustulans; Phyllachorales such as Gibellina cerealis, Phyllachora maydis and Phyllachora pomigena; Amphisphaeriales such as Griphosphaeria corticola, Lepteutypa cupressi and Pestalotia rhododendri; Capnodiales such as Capnodium ramosum and Schizothyrium pomi; Chaetothyriales such as Phialophroa spp.; Cladosporiales such as Cladosporium species including Cladosporium oxysporum; Rhytismatales such as Lophodermium seditiosum and Naemacyclus spp.; Saccharomycetales such as Cephaloascus species including Cephaloascus fragrans; Geotrichum candidum, Candida species including Candida glabrata, Candida krusei, Candida lusitaniae, Candida parapsilosis, Candida albicans and Candida tropicalis; Sordariales such as Chaetomium spp. and Monosporascus cannonballus; Sordariomycetes such as Wongia garrettii and Wongia griffinii; Taphrinales such as Taphrina bullata and Taphrina deformans; Onygenales such as Ajellomyces capsulatus, Blastomyces dermatitidis, Coccidioides species including Coccidioides immitis; Epidermophyton spp., Histoplasma spp., Microsporum spp., Trichophyton spp., and Paracoccidioides species including Paracoccioides brasiliensis; and others such as Hymenula cerealis, Petriellidum spp., and Septocyta ruborum; Basidiomycetes, including Pucciniales such as Cerotelium fici, Chrysomyxa arctostaphyli, Coleosporium ipomoeae, Cronartium ribicola, Gymnosporangium juniperi-virginianae, Gymnosporangium sabinae, Hemileia species including Hemileia vastatrix; Melampsora medusae, Melampsora lini, Phakopsora ampelopsidis, Phakopsora pachyrhizi, Phragmidium mucronatum, Puccinia species including Puccinia allii, Puccinia arachidis, Puccinia asparagi, Puccinia cacabata, Puccinia coronata, Puccinia graminis, Puccinia helianthi, Puccinia hieracii, Puccinia hordei, Puccinia horiana, Puccinia melanocephala, Puccinia polysora, Puccinia porri, Puccinia recondita, Puccinia sorghi, Puccinia striiformis, Puccinia striiformis f.sp. hordei, Puccinia striiformis f.sp. tritici and Puccinia triticina; Pucciniastrum coryli, Tranzschelia discolor, Uromyces species including Uromyces betae, Uromyces pisi and Uromyces viciae-fabae; Tilletiales such as Neovossia moliniae, and Tilletia species including Tilletia caries and Tilletia controversa; Ustilaginales such as Sporisorium reilianum and Ustilago species including Ustilago maydis, Ustilago segetum var. nuda, Ustilago segetum var. tritici and Ustilago striiformis; Urocystidales such as Urocystis species including Urocystis agropyri; Agaricales such as Marasmiellus inoderma, Mycena spp., Moniliophthora roreri and Moniliophthora perniciosa; Cantharellales such as Sclerotium spp. and Typhula species including Typhula incarnata and Typhula ishikariensis; Ceratobasidiales such as Waitea circinata, and Rhizoctonia species including Rhizoctonia cerealis, Rhizoctonia solani and Rhizoctonia theobromae; Atheliales such as Athelia rolfsii; Corticiales such as Corticium invisum and Laetisaria fuciformis; Cystodilobasidiales such as Itersonilia perplexans; Entylomatales such as Entyloma calendulae f.sp. dahliae and Entylomella microspore; Exobasidiales such as Exobasidium vexans; Hymenochaetales such as Phellinus igniarius; Russulales such as Stereum hirsutum; and Tremellales such as Cryptococcus species including Cryptococcus neoformans;
Zygomycetes, including Mucorales such as Choanephora cucurbitarum, Mucor spp., Rhizopus oryzae, Absidia corymbifera and Rhizomucor pusillus;
Blastocladiomycetes, including Physoderma maydis;
as well as diseases caused by other species and genera closely related to those listed above.

In addition to their fungicidal activity, the compounds of formula (I), and the compositions containing such compounds, may also have activity against diseases caused by Actinobacteria such as Streptomyces scabiei; Proteobacteria such as Erwinia amylovora, Pectobacterium carotovorum, Xanthomonas species including Xanthomonas axonopodis, Xanthomonas campestris, Xanthomonas oryzae and Xanthomonas vesicatoria; and Pseudomonas species including Pseudomonas syringae; Cercozoa such as Polymyxa betae, Polymyxa graminis and Spongospora subterranea; and Bigyra such as Labyrinthula zosterae.
as well as diseases caused by other species and genera closely related to those listed above.

The compounds of formula (I) may be used for example on turf, ornamentals, such as flowers, shrubs, broad-leaved trees or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g., imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CrylA(b) toxin); YieldGard Rootworm® (maize variety that expresses a CryIIIB(b1) toxin); YieldGard Plus® (maize variety that expresses a CrylA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CrylA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as d-endotoxins, e.g., Cry1Ab, Cry1Ac, Cry1F, Cry1Fa2, Cry2Ab, Cry3A, Cry3Bb1 or Cry9C, or vegetative insecticidal proteins (Vip), e.g., Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by d-endotoxins, for example Cry1Ab, Cry1Ac, Cry1F, CrylFa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known.

In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO93/07278, WO95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The compounds of formula (I) may be used in controlling or preventing phytopathogenic diseases, especially caused by phytopathogenic fungi, such as Botrytis cinerea on Rosaceae, Vitaceae, Solanaceae, Cucurbitaceae, and Fabaceae; Glomerella lagenarium on Cucurbitaceae; Sclerotinia sclerotiorum on Fabaceae, Brassicaceae, and Asteraceae, such as soybean, rapeseed, and sunflower respectively; Alternaria solani on Solanaceae, such as tomato and potato; Monographella nivalis on Poaceae; or Pyrenophora teres on Poaceae, such as barley.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula (I) may be used in unmodified form or, preferably, together with the adjuvants conventionally employed in the art of formulation. To this end they may be conveniently Formulated in known manner to emulsifiable concentrates, coatable pastes, directly sprayable or dilutable solutions or suspensions, dilute emulsions, wettable powders, soluble powders, dusts, granulates, and also encapsulations e.g., in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g., natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular Formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, divinyl glycol, divinyl glycol abietate, divinyl glycol butyl ether, divinyl glycol ethyl ether, divinyl glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, vinyl carbonate, 1,1,1-trichloroethane, 2-heptanone, alpha pinene, d-limonene, vinyl glycol, vinyl glycol butyl ether, vinyl glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, mvinyl chloride, m-xylene, n-hexane, n-octylamine, octadecanoic acid, octyl amine acetate, oleic acid, oleylamine, o-xylene, phenol, polyvinyl glycol (PEG400), propionic acid, propylene glycol, propylene glycol monomethyl ether, p-xylene, toluene, triethyl phosphate, trivinyl glycol, xylene sulfonic acid, paraffin, mineral oil, trichlorovinyl, perchlorovinyl, ethyl acetate, amyl acetate, butyl acetate, methanol, ethanol, isopropanol, and higher molecular weight alcohols such as amyl alcohol, tetrahydrofurfuryl alcohol, hexanol, octanol, etc., vinyl glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.18 ethoxylate; alcohol-alkylene oxide addition products, such as tridecyl alcohol-C.16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyvinyl glycol esters of fatty acids, such as polyvinyl glycol stearate; block copolymers of vinyl oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) are normally used in the form of agrochemical compositions and can be applied to the crop area or plant to be treated, simultaneously or in succession with further compounds.

These further compounds can be e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) may be used in the form of compositions for controlling or protecting against phytopathogenic microorganisms, comprising as active ingredient at least one compound of formula (I) or of at least one preferred individual compound as defined herein, in free form or in agrochemically usable salt form, and at least one of the above-mentioned adjuvants.

The invention therefore provides a composition, preferably a fungicidal composition, comprising at least one compound formula (I) an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally-active compounds, for example an additional fungicidal active ingredient in addition to the compound of formula (I).

The following mixtures of the compounds of formula (I) with active ingredients are preferred (where the abbreviation “TX” means “one compound selected from the compounds defined in the Tables A-1 to A-17, and their subtables, or a compound selected from P-1.1 to P-1.19 listed in Table T1 (below)”): a compound selected from the group of substances consisting of petroleum oils+TX, 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chinomethionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dinopenton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fenpyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure Bi+TX, trimedlure B₂+TX, trimedlure C+TX, trunc-call+TX, 2-(octylthio)ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, 0-5-dichloro-4-iodophenyl O-ethyl ethylphosphonothioate+TX, 0,0-diethyl 0-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imibenconazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, −simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl+TX, R-metalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorotha-Ionil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)−N-(2-chloro-6-fluorophenyl)−1,3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-oI+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate+TX (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate+TX (may be prepared from the methods described in WO 2020/056090), methyl N-[[4-[1-(4-cyclopropyl-2,6-difluoro-phenyl)pyrazol-4-yl]-2-methyl-phenyl]methyl]carbamate+TX (may be prepared from the methods described in WO 2020/097012), methyl N-[[4-[1-(2,6-difluoro-4-isopropyl-phenyl)pyrazol-4-yl]-2-methyl-phenyl]methyl]carbamate+TX (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+TX (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+TX (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+TX (may be prepared from the methods described in WO 2020/109391), N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)−N—((3R)−1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)−N—((3R)−1, 1, 3-trimethylindan-4-yl) pyridine-3-carboxamide+TX, α-(1, 1-dimethylethyl)−α-[4′-(trifluoromethoxy) [1,1′-biphenyl]-4-yl]-5-pyrimidinemethanol+TX, fluoxapiprolin+TX, enoxastrobin+TX, methyl (Z)-3-methoxy-2-[2-methyl-5-[4-(trifluoromethyl)triazol-2-yl]phenoxy]prop-2-enoate+TX, methyl (Z)-3-methoxy-2-[2-methyl-5-(4-propyltriazol-2-yl)phenoxy]prop-2-enoate+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-[3-(trifluoromethyl)pyrazol-1-yl]phenoxy]prop-2-enoate+TX (these compounds may be prepared from the methods described in WO2020/079111), methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate+TX (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N—[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428).

The references in brackets behind the active ingredients, e.g., [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula (I) is selected from one compound as represented in Tables A-1 to A-17, and their subtables (below), or a compound selected from P-1.1 to P-1.19 listed in Table T1 (below) is preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound as represented in Tables A-1 to A-17, and their subtables (below), or a compound P-1.1 to P-1.19 listed in Table T1 (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying a compound as represented in Tables A-1 to A-17, and their subtables (below), or a compound P-1.1 to P-1.19 listed in Table T1 (below) and the active ingredient(s) as described above, is not essential for working the present invention.

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291.

Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO 96/15121 and also with anthelmintic active cyclic depsipeptides such as those described in WO 96/11945, WO 93/19053, WO 93/25543, EP 0 626 375, EP 0 382 173, WO 94/19334, EP 0 382 173, and EP 0 503 538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO 95/19363 or WO 04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195, RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of the invention is related to the use of a compound of formula (I) or of a preferred individual compound as defined herein, of a composition comprising at least one compound of formula (I) or at least one preferred individual compound as above-defined, or of a fungicidal or insecticidal mixture comprising at least one compound of formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of the invention is related to a method of controlling or preventing an infestation of plants, e.g., useful plants such as crop plants, propagation material thereof, e.g. seeds, harvested crops, e.g., harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of Formula (I), or an agrochemical composition which contains at least one of said compounds, is foliar application. The frequency of application and the rate of application will depend on the risk of infestation by the corresponding pathogen or insect. However, the compounds of Formula (I) can also penetrate the plant through the roots via the soil (systemic action) by drenching the locus of the plant with a liquid Formulation, or by applying the compounds in solid form to the soil, e.g. in granular form (soil application).

In crops of water rice such granulates can be applied to the flooded rice field. The compounds of Formula (I) may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of formula (I) per kg of seed, preferably from 0.01 to 10 g per kg of seed are generally sufficient.

Suitably, a composition comprising a compound of formula (I) according to the present invention is applied either preventative, meaning priorto disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly Formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of Formula (I) optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

The compounds according to the following Tables A-1 to A-17 below can be prepared following the methods described above. The examples which follow are intended to illustrate the invention and show preferred compounds of formula (Ia).

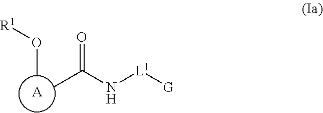

Table A: Compounds of the formula (Ia) wherein R¹, R¹, R⁸ and R⁹ are defined in Tables A-1 to A-17, and NH-L¹-G is as defined in Table Z below

TABLE Z

| | -NH-L¹-G |
|---|---|
| 1 | (2,4-dimethylphenyl)methylhydrazino |
| 2 | (2,4-dichlorophenyl)methylhydrazino |
| 3 | 1-aminotetralin-6-carbonitrile |
| 4 | 5-methoxytetralin-1-amino |
| 5 | 7-chlorochroman-4-amino |
| 6 | (1S)-4,6-dichloroindan-1-amino |
| 7 | 1-aminoindane-5-carbonitrile |
| 8 | 5,7-dichloroindan-1-amino |
| 9 | O-[(2,4- |
| | dichlorophenyl)methyl]hydroxylamino |
| 10 | 3-(2,4-dichlorophenyl)propan-1-amino |
| 11 | 2-(2,4-dimethylphenyl)-2-fluoro- |
| | ethylamino |
| 12 | 2-(2,4-dichlorophenyl)-2,2-difluoro- |
| | ethylamino |
| 13 | 2-(2,4-difluorophenyl)-2,2-difluoro- |
| | ethylamino |
| 14 | 2-(2,4-dichlorophenyl)-2-fluoro-ethylamino |
| 15 | (2R)-2-(2,4-dichlorophenyl)-2-fluoro- |
| | ethylamino |
| 16 | (2S)-2-(2,4-dichlorophenyl)-2-fluoro- |
| | ethylamino |
| 17 | 2-(2,4-dichlorophenyl)-2-fluoro-propan-1- |
| | amino |
| 18 | [1-(2,4- |
| | dichlorophenyl)cyclopropyl]methylamino |
| 19 | 2-(4,6-dichloro-3-pyridyl)-2-fluoro- |
| | ethylamino |
| 20 | 2-(2-chloro-4-methoxy-phenyl)-2-fluoro- |
| | ethylamino |
| 21 | 2-fluoro-2-(4-methoxy-2-methyl- |
| | phenyl)ethylamino |
| 22 | 2-(3,5-dichloro-2-pyridyl)-2-fluoro- |
| | ethylamino |
| 23 | 2-(2,4-dichlorophenyl)ethylamino |

Table A-1: Compounds of the formula (Ia-A-1) wherein R¹, R¹, R⁸ and R⁹ are defined in Tables A-1a to A-1j, and NH-L¹-G is as defined in Table Z above:

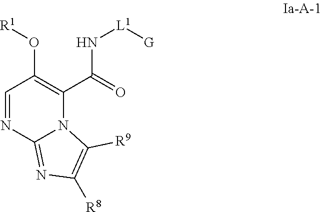

Table A-1a: This subtable provides 23 compounds A-1a.01 to A-1a.23 of formula (Ia-A-1) wherein R¹ is 3-cyclopropyl-2-fluorophenyl, R⁸ and R⁹ are hydrogen and substituents NH-L¹-G are as defined in Table Z above.

Table A-1b: This subtable provides 23 compounds A-1 b.01 to A-1 b.23 of formula (Ia-A-1) wherein R¹ is 3-(trifluoromethyl)phenyl, R⁸ and R⁹ are hydrogen and substituents NH-L¹-G are as defined in Table Z above.

Table A-1c: This subtable provides 23 compounds A-1c.01 to A-1c.23 of formula (Ia-A-1) wherein R¹ is 3-ethynylphenyl, R⁸ and R⁹ are hydrogen and substituents NH-L¹-G are as defined in Table Z above.

Table A-1d: This subtable provides 23 compounds A-1d.01 to A-1d.23 of formula (Ia-A-1) wherein R¹ is 3-chlorophenyl, R⁸ and R⁹ are hydrogen and substituents NH-L¹-G are as defined in Table Z above.

Table A-1e: This subtable provides 23 compounds A-1e.01 to A-1e.23 of formula (Ia-A-1) wherein R¹ is 5-cyanopyridin-3-yl, R⁸ and R⁹ are hydrogen and substituents NH-L¹-G are as defined in Table Z above.

Table A-1f: This subtable provides 23 compounds A-1f.01 to A-1f.23 of formula (Ia-A-1) wherein R¹ is 3-cyclopropylphenyl, R⁸ and R⁹ are hydrogen and substituents NH-L¹-G are as defined in Table Z above.

Table A-1q: This subtable provides 23 compounds A-1 g.01 to A-1 g.23 of formula (Ia-A-1) wherein R¹ is 3-cyclopropyl-2-fluorophenyl, R⁸ is methyl and R⁹ is hydrogen and substituents NH-L¹-G are as defined in Table Z above.

Table A-1h: This subtable provides 23 compounds A-1h.01 to A-1h.23 of formula (Ia-A-1) wherein R¹ is 3-(trifluoromethyl)phenyl, R⁸ is chloro and R⁹ is hydrogen and substituents NH-L¹-G are as defined in Table Z above.

Table A-1i: This subtable provides 23 compounds A-1i.01 to A-1i.23 of formula (Ia-A-1) wherein R¹ is 5-cyanopyridin-3-yl, R⁸ is hydrogen and R⁹ is chloro and substituents NH-L¹-G are as defined in Table Z above.

Table A-1j: This subtable provides 23 compounds A-1j.01 to A-1j.23 of formula (Ia-A-1) wherein R¹ is 3-cyclopropylphenyl, R⁸ is methyl and R⁹ is hydrogen and substituents NH-L¹-G are as defined in Table Z above.

Table A-2: Compounds of the formula (Ia-A-2) wherein R¹, R¹, R⁸ and R⁹ are defined in Tables A-2a to A-2k, and NH-L1-G is as defined in Table Z above:

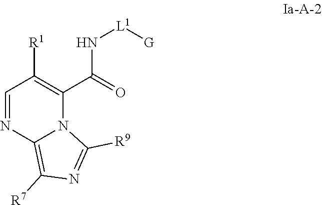

Table A-2a: This subtable provides 23 compounds A-2a.01 to A-2a.23 of formula (Ia-A-2) wherein R¹ is 3-cyclopropyl-2-fluorophenyl, R⁷ and R⁹ are hydrogen and substituents NH-L¹-G are as defined in Table Z above.

As an example, compound A-2a.01 is:

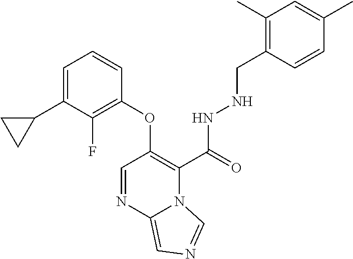 [SMILES: Cc1ccc(CNNC(=O)c2c(Oc3cccc(C4CC4)c3F)cnc3cncn23)c(C)c1]

Table A-2b: This subtable provides 23 compounds A-2b.01 to A-2b.23 of formula (Ia-A-2) wherein R¹ is 3-(trifluoromethyl)phenyl, R⁷ and R⁹ are hydrogen and substituents NH-L¹-G are as defined in Table Z above.

Table A-2c: This subtable provides 23 compounds A-2c.01 to A-2c.23 of formula (Ia-A-2) wherein R¹ is 3-ethynylphenyl, R⁷ and R⁹ are hydrogen and substituents NH-L¹-G are as defined in Table Z above.

Table A-2d: This subtable provides 23 compounds A-2d.01 to A-2d.23 of formula (Ia-A-2) wherein R¹ is 3-chlorophenyl, R⁷ and R⁹ are hydrogen and substituents NH-L¹-G are as defined in Table Z above.

Table A-2e: This subtable provides 23 compounds A-2e.01 to A-2e.23 of formula (Ia-A-2) wherein R¹ is 5-cyanopyridin-3-yl, R⁷ and R⁹ are hydrogen and substituents NH-L¹-G are as defined in Table Z above.

Table A-2f: This subtable provides 23 compounds A-2f.01 to A-2f.23 of formula (Ia-A-2) wherein R¹ is 5-(cyclopropyl)pyridin-3-yl, R⁷ and R⁹ are hydrogen and substituents NH-L¹-G are as defined in Table Z above.

Table A-2q: This subtable provides 23 compounds A-2 g.01 to A-2 g.23 of formula (Ia-A-2) wherein R¹ is 3-cyclopropylphenyl, R⁷ and R⁹ are hydrogen and substituents NH-L¹-G are as defined in Table Z above.

Table A-2h: This subtable provides 23 compounds A-2h.01 to A-2h.23 of formula (Ia-A-2) wherein R¹ is 3-cyclopropyl-2-fluorophenyl, R⁷ is hydrogen R⁹ is methyl and substituents NH-L¹-G are as defined in Table Z above.

Table A-2i: This subtable provides 23 compounds A-2i.01 to A-2i.23 of formula (Ia-A-2) wherein R¹ is 3-(trifluoromethyl)phenyl, R⁷ is hydrogen R⁹ is methyl and substituents NH-L¹-G are as defined in Table Z above.

Table A-2j: This subtable provides 23 compounds A-2j.01 to A-2j.23 of formula (Ia-A-2) wherein R¹ is 5-cyanopyridin-3-yl, R⁷ is hydrogen R⁹ is methyl and substituents NH-L¹-G are as defined in Table Z above.

Table A-2k: This subtable provides 23 compounds A-2k.01 to A-2k.23 of formula (Ia-A-2) wherein R¹ is 3-cyclopropylphenyl, R⁷ is hydrogen R⁹ is methyl and substituents NH-L¹-G are as defined in Table Z above.

Table A-3: Compounds of the formula (Ia-A-3) wherein R¹, R¹, R⁸ and R⁹ are defined in Tables A-3a to A-3k, and NH-L¹-G is as defined in Table Z above:

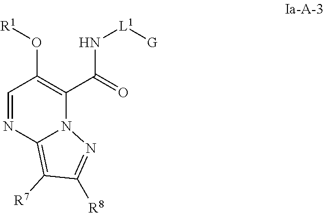

Table A-3a: This subtable provides 23 compounds A-3a.01 to A-3a.23 of formula (Ia-A-3) wherein R¹ is 3-cyclopropyl-2-fluorophenyl, R⁷ and R⁸ are hydrogen and substituents NH-L¹-G are as defined in Table Z above.

Table A-3b: This subtable provides 23 compounds A-3b.01 to A-3b.23 of formula (Ia-A-3) wherein R¹ is 3-(trifluoromethyl)phenyl, R⁷ and R⁸ are hydrogen and substituents NH-L¹-G are as defined in Table Z above.

Table A-3c: This subtable provides 23 compounds A-3c.01 to A-3c.23 of formula (Ia-A-3) wherein R¹ is 3-ethynylphenyl, R⁷ and R⁸ are hydrogen and substituents NH-L¹-G are as defined in Table Z above.

Table A-3d: This subtable provides 23 compounds A-3d.01 to A-3d.23 of formula (Ia-A-3) wherein R¹ is 3-chlorophenyl, R⁷ and R⁸ are hydrogen and substituents NH-L¹-G are as defined in Table Z above.

Table A-3e: This subtable provides 23 compounds A-3e.01 to A-3e.23 of formula (Ia-A-3) wherein R¹ is 5-cyanopyridin-3-yl, R⁷ and R⁸ are hydrogen and substituents NH-L¹-G are as defined in Table Z above.

Table A-3f: This subtable provides 23 compounds A-3f.01 to A-3f.23 of formula (Ia-A-3) wherein R¹ is 5-(cyclopropyl)pyridin-3-yl, R⁷ and R⁸ are hydrogen and substituents NH-L¹-G are as defined in Table Z above.

Table A-3q: This subtable provides 23 compounds A-3 g.01 to A-3 g.23 of formula (Ia-A-3) wherein R¹ is 3-cyclopropylphenyl, R⁷ and R⁸ are hydrogen and substituents NH-L¹-G are as defined in Table Z above.

Table A-3h: This subtable provides 23 compounds A-3h.01 to A-3h.23 of formula (Ia-A-3) wherein R¹ is 3-cyclopropyl-2-fluorophenyl, R⁷ is methyl and R⁸ are hydrogen and substituents NH-L¹-G are as defined in Table Z above.

Table A-3i: This subtable provides 23 compounds A-3i.01 to A-3i.23 of formula (Ia-A-3) wherein R¹ is 3-(trifluoromethyl)phenyl, R⁷ is chloro and R⁸ are hydrogen and substituents NH-L¹-G are as defined in Table Z above.

As an example, compound A-3i-15 is:

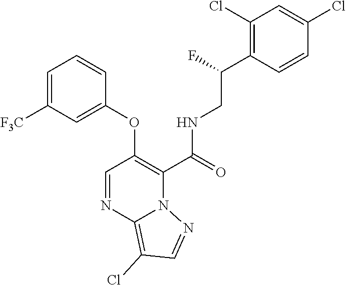 [SMILES: O=C(NC[C@H](F)c1ccc(Cl)cc1Cl)c1c(Oc2cccc(C(F)(F)F)c2)cnc2c(Cl)cnn12]

Table A-3j: This subtable provides 23 compounds A-3j.01 to A-3j.23 of formula (Ia-A-3) wherein R¹ is 5-cyanopyridin-3-yl, R⁷ hydrogen and R⁸ is chloro and substituents NH-L¹-G are as defined in Table Z above.

Table A-3k: This subtable provides 23 compounds A-3k.01 to A-3k.23 of formula (Ia-A-3) wherein R¹ is 3-cyclopropylphenyl, R⁷ hydrogen and R⁸ is fluoro and substituents NH-L¹-G are as defined in Table Z above.

Table A-4: Compounds of the formula (Ia-A-4) wherein R¹, R¹, R⁸ and R⁹ are defined in Tables A-4a to A-4d, and NH-L¹-G is as defined in Table Z above:

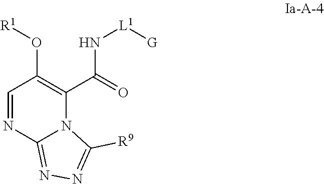

Table A-4a: This subtable provides 23 compounds A-4a.01 to A-4a.23 of formula (Ia-A-4) wherein R¹ is 3-cyclopropyl-2-fluorophenyl, R⁹ is hydrogen and substituents NH-L¹-G are as defined in Table Z above.

Table A-4b: This subtable provides 23 compounds A-4b.01 to A-4b.23 of formula (Ia-A-4) wherein R¹ is 3-(trifluoromethyl)phenyl, R⁹ is hydrogen and substituents NH-L¹-G are as defined in Table Z above.

Table A-4c: This subtable provides 23 compounds A-4c.01 to A-4c.23 of formula (Ia-A-4) wherein R¹ is 5-cyanopyridin-3-yl, R⁹ is hydrogen and substituents NH-L¹-G are as defined in Table Z above.

Table A-4d: This subtable provides 23 compounds A-4d.01 to A-4d.23 of formula (Ia-A-4) wherein R¹ is 3-cyclopropylphenyl, R⁹ is hydrogen and substituents NH-L¹-G are as defined in Table Z above.

Table A-5: Compounds of the formula (Ia-A-5) wherein R¹, R¹, R⁸ and R⁹ are defined in Tables A-5a to A-5d, and NH-L¹-G is as defined in Table Z above:

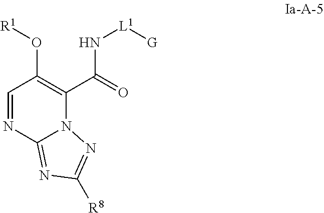

Table A-5a: This subtable provides 23 compounds A-5a.01 to A-5a.23 of formula (Ia-A-5) wherein R¹ is 3-cyclopropyl-2-fluorophenyl, R⁸ is hydrogen and substituents NH-L¹-G are as defined in Table Z above.

Table A-5b: This subtable provides 23 compounds A-5b.01 to A-5b.23 of formula (Ia-A-5) wherein R¹ is 3-(trifluoromethyl)phenyl, R⁸ is hydrogen and substituents NH-L¹-G are as defined in Table Z above.

Table A-5c: This subtable provides 23 compounds A-5c.01 to A-5c.23 of formula (Ia-A-5) wherein R¹ is 5-(cyclopropyl)pyridin-3-yl, R⁸ is hydrogen and substituents NH-L¹-G are as defined in Table Z above.

Table A-5d: This subtable provides 23 compounds A-5d.01 to A-5d.23 of formula (Ia-A-5) wherein R¹ is 3-cyclopropylphenyl, R⁸ is hydrogen and substituents NH-L¹-G are as defined in Table Z above.

Table A-6: Compounds of the formula (Ia-A-6) wherein R¹, R¹, R⁸ and R⁹ are defined in Tables A-6a to A-6c, and NH-L¹-G is as defined in Table Z above:

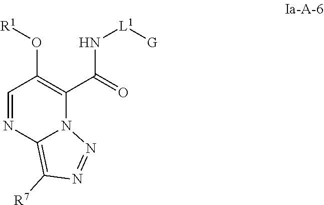

Table A-6a: This subtable provides 23 compounds A-6a.01 to A-6a.23 of formula (Ia-A-6) wherein R¹ is 3-cyclopropyl-2-fluorophenyl, R⁷ is hydrogen and substituents NH-L¹-G are as defined in Table Z above.

Table A-6b: This subtable provides 23 compounds A-6b.01 to A-6b.23 of formula (Ia-A-6) wherein R¹ is 3-(trifluoromethyl)phenyl, R⁷ is hydrogen and substituents NH-L¹-G are as defined in Table Z above.

Table A-6c: This subtable provides 23 compounds A-6c.01 to A-6c.23 of formula (Ia-A-6) wherein R¹ is 3-cyclopropylphenyl, R⁷ is hydrogen and substituents NH-L¹-G are as defined in Table Z above.

As an example, compound A-6c.02 is:

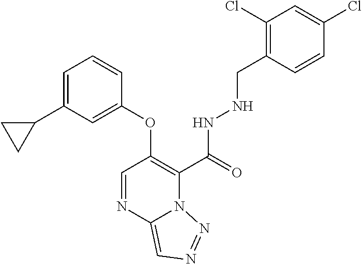 [SMILES: O=C(NNCc1ccc(Cl)cc1Cl)c1c(Oc2cccc(C3CC3)c2)cnc2cnnn12]

Table A-7: Compounds of the formula (Ia-A-7) wherein R¹, R¹, R⁸ and R⁹ are defined in Tables A-7a to A-7f, and NH-L¹-G is as defined in Table Z above:

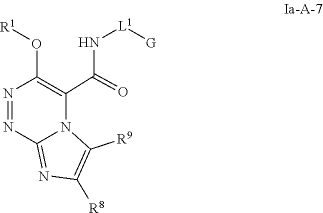

Table A-7a: This subtable provides 23 compounds A-7a.01 to A-7a.23 of formula (Ia-A-7) wherein R¹ is 3-cyclopropyl-2-fluorophenyl, R⁸ and R⁹ are hydrogen and substituents NH-L¹-G are as defined in Table Z above.

Table A-7b: This subtable provides 23 compounds A-7b.01 to A-7b.23 of formula (Ia-A-7) wherein R¹ is 3-(trifluoromethyl)phenyl, R⁸ and R⁹ are hydrogen and substituents NH-L¹-G are as defined in Table Z above.

Table A-7c: This subtable provides 23 compounds A-7c.01 to A-7c.23 of formula (Ia-A-7) wherein R¹ is 3-cyclopropylphenyl, R⁸ and R⁹ are hydrogen and substituents NH-L¹-G are as defined in Table Z above.

Table A-7d: This subtable provides 23 compounds A-7d.01 to A-7d.23 of formula (Ia-A-7) wherein R¹ is 3-cyclopropyl-2-fluorophenyl, R⁸ is chloro and R⁹ is hydrogen and substituents NH-L¹-G are as defined in Table Z above.

As an example, compound A-7d.03 is:

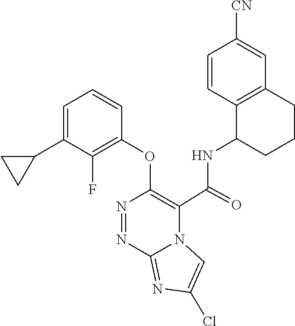 [SMILES: N#Cc1ccc2c(c1)CCCC2NC(=O)c1c(Oc2cccc(C3CC3)c2F)nnc2nc(Cl)cn12]

Table A-7e: This subtable provides 23 compounds A-7e.01 to A-7e.23 of formula (Ia-A-7) wherein R¹ is 3-(trifluoromethyl)phenyl, R⁸ is hydrogen and R⁹ is chloro and substituents NH-L¹-G are as defined in Table Z above.

Table A-7f: This subtable provides 23 compounds A-7f.01 to A-7f.23 of formula (Ia-A-7) wherein R¹ is 3-cyclopropylphenyl, R⁸ is methyl and R⁹ is hydrogen and substituents NH-L¹-G are as defined in Table Z above.

Table A-8: Compounds of the formula (Ia-A-8) wherein R¹, R¹, R⁸ and R⁹ are defined in Tables A-8a to A-8h, and NH-L¹-G is as defined in Table Z above:

as

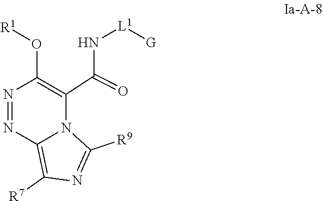

Table A-8a: This subtable provides 23 compounds A-8a.01 to A-8a.23 of formula (Ia-A-8) wherein R¹ is 3-cyclopropyl-2-fluorophenyl, R⁷ and R⁹ are hydrogen and substituents NH-L¹-G are as defined in Table Z above.

Table A-8b: This subtable provides 23 compounds A-8b.01 to A-8b.23 of formula (Ia-A-8) wherein R¹ is 3-(trifluoromethyl)phenyl, R⁷ and R⁹ are hydrogen and substituents NH-L¹-G are as defined in Table Z above.

Table A-8c: This subtable provides 23 compounds A-8c.01 to A-8c.23 of formula (Ia-A-8) wherein R¹ is 5-cyanopyridin-3-yl, R⁷ and R⁹ are hydrogen and substituents NH-L¹-G are as defined in Table Z above.

Table A-8d: This subtable provides 23 compounds A-8d.01 to A-8d.23 of formula (Ia-A-8) wherein R¹ is 5-(cyclopropyl)pyridin-3-yl, R⁷ and R⁹ are hydrogen and substituents NH-L¹-G are as defined in Table Z above.

Table A-8e: This subtable provides 23 compounds A-8e.01 to A-8e.23 of formula (Ia-A-8) wherein R¹ is 3-cyclopropylphenyl, R⁷ and R⁹ are hydrogen and substituents NH-L¹-G are as defined in Table Z above.

Table A-8f: This subtable provides 23 compounds A-8f.01 to A-8f.23 of formula (Ia-A-8) wherein R¹ is 3-cyclopropyl-2-fluorophenyl, R⁷ is hydrogen R⁹ is methyl and substituents NH-L¹-G are as defined in Table Z above.

Table A-8 g: This subtable provides 23 compounds A-8 g.01 to A-8 g.23 of formula (Ia-A-8) wherein R¹ is 3-(trifluoromethyl)phenyl, R⁷ is hydrogen R⁹ is methyl and substituents NH-L¹-G are as defined in Table Z above.

Table A-8h: This subtable provides 23 compounds A-8h.01 to A-8h.23 of formula (Ia-A-8) wherein R¹ is 3-cyclopropylphenyl, R⁷ is hydrogen R⁹ is methyl and substituents NH-L¹-G are as defined in Table Z above.

As an example, compound A-8h.22 is:

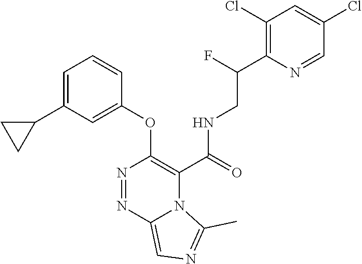 [SMILES: Cc1ncc2nnc(Oc3cccc(C4CC4)c3)c(C(=O)NCC(F)c3ncc(Cl)cc3Cl)n12]

Table A-9: Compounds of the formula (Ia-A-9) wherein R¹, R¹, R⁸ and R⁹ are defined in Tables A-9a to A-9h, and NH-L¹-G is as defined in Table Z above:

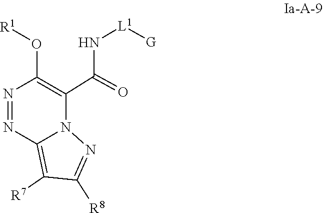

Table A-9a: This subtable provides 23 compounds A-9a.01 to A-9a.23 of formula (Ia-A-9) wherein R¹ is 3-cyclopropyl-2-fluorophenyl, R⁷ and R⁸ are hydrogen and substituents NH-L¹-G are as defined in Table Z above.

Table A-9b: This subtable provides 23 compounds A-9b.01 to A-9b.23 of formula (Ia-A-9) wherein R¹ is 3-(trifluoromethyl)phenyl, R⁷ and R⁸ are hydrogen and substituents NH-L¹-G are as defined in Table Z above.

Table A-9c: This subtable provides 23 compounds A-9c.01 to A-9c.23 of formula (Ia-A-9) wherein R¹ is 5-cyanopyridin-3-yl, R⁷ and R⁸ are hydrogen and substituents NH-L¹-G are as defined in Table Z above.

As an example, compound A-9c.19 is:

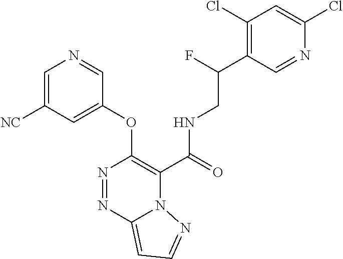 [SMILES: N#Cc1cncc(Oc2nnc3ccnn3c2C(=O)NCC(F)c2cnc(Cl)cc2Cl)c1]

Table A-9d: This subtable provides 23 compounds A-9d.01 to A-9d.23 of formula (Ia-A-9) wherein R¹ is 3-cyclopropylphenyl, R⁷ and R⁸ are hydrogen and substituents NH-L¹-G are as defined in Table Z above.

Table A-9e: This subtable provides 23 compounds A-9e.01 to A-9e.23 of formula (Ia-A-9) wherein R¹ is 3-cyclopropyl-2-fluorophenyl, R⁷ is chloro and R⁸ are hydrogen and substituents NH-L¹-G are as defined in Table Z above.

Table A-9f: This subtable provides 23 compounds A-9f.01 to A-9f.23 of formula (Ia-A-9) wherein R¹ is 3-(trifluoromethyl)phenyl, R⁷ hydrogen and R⁸ is chloro and substituents NH-L¹-G are as defined in Table Z above.

Table A-9q: This subtable provides 23 compounds A-9 g.01 to A-9 g.23 of formula (Ia-A-9) wherein R¹ is 5-cyanopyridin-3-yl, R⁷ hydrogen and R⁸ is fluoro and substituents NH-L¹-G are as defined in Table Z above.

Table A-9h: This subtable provides 23 compounds A-9h.01 to A-9h.23 of formula (Ia-A-9) wherein R¹ is 3-cyclopropylphenyl, R⁷ hydrogen and R⁸ is methyl and substituents NH-L¹-G are as defined in Table Z above.

Table A-10: Compounds of the formula (Ia-A-10) wherein R¹, R¹, R⁸ and R⁹ are defined in Tables A-10a to A-10c, and NH-L¹-G is as defined in Table Z above:

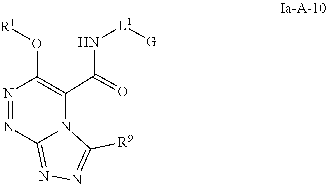

Table A-10a: This subtable provides 23 compounds A-10a.01 to A-10a.23 of formula (Ia-A-10) wherein R¹ is 3-cyclopropyl-2-fluorophenyl, R⁹ is hydrogen and substituents NH-L¹-G are as defined in Table Z above.

Table A-10b: This subtable provides 23 compounds A-10b.01 to A-10b.23 of formula (Ia-A-10) wherein R¹ is 3-(trifluoromethyl)phenyl, R⁹ is hydrogen and substituents NH-L¹-G are as defined in Table Z above.

As an example, compound A-10b.21 is:

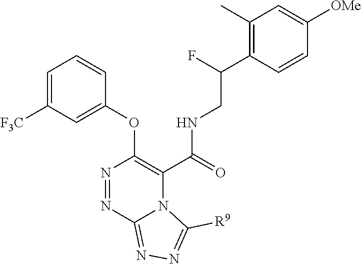

Table A-10c: This subtable provides 23 compounds A-10c.01 to A-10c.23 of formula (Ia-A-10) wherein R¹ is 3-cyclopropylphenyl, R⁹ is hydrogen and substituents NH-L¹-G are as defined in Table Z above.

Table A-11: Compounds of the formula (Ia-A-11-1) wherein R¹, R⁷, R⁸ and R⁹ are defined in Tables A-11a to A-11c, and NH-L¹-G is as defined in Table Z above:

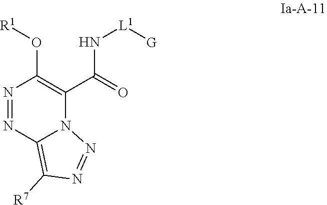

Table A-11a: This subtable provides 23 compounds A-11a.01 to A-11a.23 of formula (Ia-A-11) wherein R¹ is 3-cyclopropyl-2-fluorophenyl, R⁷ is hydrogen and substituents NH-L¹-G are as defined in Table Z above.

Table A-11 b: This subtable provides 23 compounds A-11 b.01 to A-11 b.23 of formula (Ia-A-11) wherein R¹ is 3-(trifluoromethoxy)phenyl, R⁷ is hydrogen and substituents NH-L¹-G are as defined in Table Z above.

Table A-11c: This subtable provides 23 compounds A-11c.01 to A-11c.23 of formula (Ia-A-11) wherein R¹ is 3-cyclopropylphenyl, R⁷ is hydrogen and substituents NH-L¹-G are as defined in Table Z above.

Table A-12: Compounds of the formula (Ia-A-12) wherein R¹, R¹, R⁸ and R⁹ are defined in Tables A-12a to A-12c, and NH-L¹-G is as defined in Table Z above:

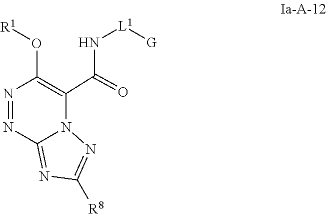

Table A-12a: This subtable provides 23 compounds A-12a.01 to A-12a.23 of formula (Ia-A-12) wherein R¹ is 3-cyclopropyl-2-fluorophenyl, R⁸ is hydrogen and substituents NH-L¹-G are as defined in Table Z above.

Table A-12b: This subtable provides 23 compounds A-12b.01 to A-12b.23 of formula (Ia-A-12) wherein R¹ is 3-(trifluoromethyl)phenyl, R⁸ is hydrogen and substituents NH-L¹-G are as defined in Table Z above.

Table A-12c: This subtable provides 23 compounds A-12c.01 to A-12c.23 of formula (Ia-A-12) wherein R¹ is 3-cyclopropylphenyl, R⁸ is hydrogen and substituents NH-L¹-G are as defined in Table Z above.

Table A-13: Compounds of the formula (Ia-A-13) wherein R¹, R¹, R⁸ and R⁹ are defined in Table A-13a and NH-L¹-G is as defined in Table Z above:

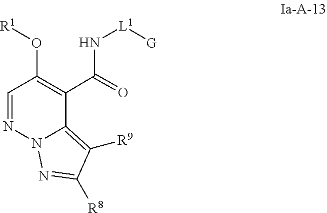

Table A-13a: This subtable provides 23 compounds A-13a.01 to A-13a.23 of formula (Ia-A-13) wherein R¹ is 3-cyclopropyl-2-fluorophenyl, R⁸ and R⁹ are hydrogen and substituents NH-L¹-G are as defined in Table Z above.

Table A-14: Compounds of the formula (Ia-A-14) wherein R¹, R¹, R⁸ and R⁹ are defined in Tables A-14a to A-14f, and NH-L¹-G is as defined in Table Z above:

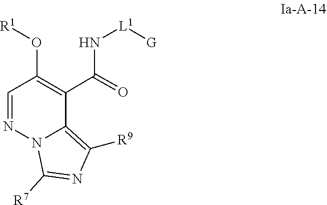

Table A-14a: This subtable provides 23 compounds A-14a.01 to A-14a.23 of formula (Ia-A-14) wherein R¹ is 3-cyclopropyl-2-fluorophenyl, R⁷ and R⁹ are hydrogen and substituents NH-L¹-G are as defined in Table Z above.

Table A-14b: This subtable provides 23 compounds A-14b.01 to A-14b.23 of formula (Ia-A-14) wherein R¹ is 3-(trifluoromethyl)phenyl, R⁷ and R⁹ are hydrogen and substituents NH-L¹-G are as defined in Table Z above.

Table A-14c: This subtable provides 23 compounds A-14c.01 to A-14c.23 of formula (Ia-A-14) wherein R¹ is 5-cyanopyridin-3-yl, R⁷ and R⁹ are hydrogen and substituents NH-L¹-G are as defined in Table Z above.

Table A-14d: This subtable provides 23 compounds A-14d.01 to A-14d.23 of formula (Ia-A-14) wherein R¹ is 3-cyclopropylphenyl, R⁷ and R⁹ are hydrogen and substituents NH-L¹-G are as defined in Table Z above.

Table A-14e: This subtable provides 23 compounds A-14e.01 to A-14e.23 of formula (Ia-A-14) wherein R¹ is 3-cyclopropyl-2-fluorophenyl, R⁷ is hydrogen R⁹ is methyl and substituents NH-L¹-G are as defined in Table Z above.

Table A-14f: This subtable provides 23 compounds A-14f.01 to A-14f.23 of formula (Ia-A-14) wherein R¹ is 3-(trifluoromethyl)phenyl, R⁷ is hydrogen R⁹ is methyl and substituents NH-L¹-G are as defined in Table Z above.

Table A-15: Compounds of the formula (Ia-A-15) wherein R¹, R¹, R⁸ and R⁹ are defined in Tables A-15a to A-15f, and NH-L¹-G is as defined in Table Z above:

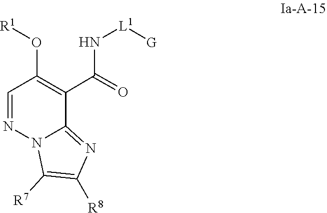

Table A-15a: This subtable provides 23 compounds A-15a.01 to A-15a.23 of formula (Ia-A-15) wherein R¹ is 3-cyclopropyl-2-fluorophenyl, R⁷ and R⁸ are hydrogen and substituents NH-L¹-G are as defined in Table Z above.

Table A-15b: This subtable provides 23 compounds A-15b.01 to A-15b.23 of formula (Ia-A-15) wherein R¹ is 3-(trifluoromethyl)phenyl, R⁷ and R⁸ are hydrogen and substituents NH-L¹-G are as defined in Table Z above.

Table A-15c: This subtable provides 23 compounds A-15c.01 to A-15c.23 of formula (Ia-A-15) wherein R¹ is 5-cyanopyridin-3-yl, R⁷ and R⁸ are hydrogen and substituents NH-L¹-G are as defined in Table Z above.

Table A-15d: This subtable provides 23 compounds A-15d.01 to A-15d.23 of formula (Ia-A-15) wherein R¹ is 5-(cyclopropyl)pyridin-3-yl, R⁷ and R⁸ are hydrogen and substituents NH-L¹-G are as defined in Table Z above.

Table A-15e: This subtable provides 23 compounds A-15e.01 to A-15e.23 of formula (Ia-A-15) wherein R¹ is 3-cyclopropyl-2-fluorophenyl, R⁷ is chloro and R⁸ are hydrogen and substituents NH-L¹-G are as defined in Table Z above.

Table A-15f: This subtable provides 23 compounds A-15f.01 to A-15f.23 of formula (Ia-A-15) wherein R¹ is 3-cyclopropylphenyl, R⁷ is methyl and R⁸ are hydrogen and substituents NH-L¹-G are as defined in Table Z above.

Table A-16: Compounds of the formula (Ia-A-16) wherein R¹, R¹, R⁸ and R⁹ are defined in Tables A-16a to A-16e, and NH-L¹-G is as defined in Table Z above:

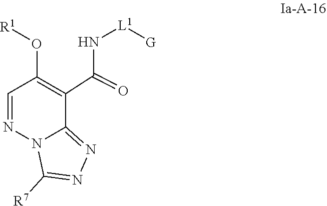

Table A-16a: This subtable provides 23 compounds A-16a.01 to A-16a.23 of formula (Ia-A-16) wherein R¹ is 3-cyclopropyl-2-fluorophenyl, R⁷ is hydrogen and substituents NH-L¹-G are as defined in Table Z above.

Table A-16b: This subtable provides 23 compounds A-16b.01 to A-16b.23 of formula (Ia-A-16) wherein R¹ is 3-(trifluoromethyl)phenyl, R⁷ is hydrogen and substituents NH-L¹-G are as defined in Table Z above.

Table A-16c: This subtable provides 23 compounds A-16c.01 to A-16c.23 of formula (Ia-A-16) wherein R¹ is 5-cyanopyridin-3-yl, R⁷ is hydrogen and substituents NH-L¹-G are as defined in Table Z above.

Table A-16d: This subtable provides 23 compounds A-16d.01 to A-16d.23 of formula (Ia-A-16) wherein R¹ is 5-(cyclopropyl)pyridin-3-yl, R⁷ is hydrogen and substituents NH-L¹-G are as defined in Table Z above.

Table A-16e: This subtable provides 23 compounds A-16e.01 to A-16e.23 of formula (Ia-A-16) wherein R¹ is 3-cyclopropylphenyl, R⁷ is hydrogen and substituents NH-L¹-G are as defined in Table Z above.

Table A-17: Compounds of the formula (Ia-A-17) wherein R¹, R⁷, R⁸ and R⁹ are defined in Tables A-17a to A-17c, and NH-L¹-G is as defined in Table Z above:

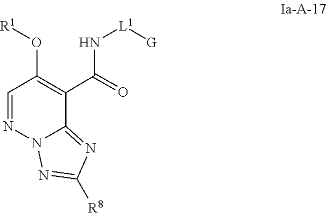

Table A-17a: This subtable provides 23 compounds A-17a.01 to A-17a.23 of formula (Ia-A-17) wherein R¹ is 3-cyclopropyl-2-fluorophenyl, R⁸ is hydrogen and substituents NH-L¹-G are as defined in Table Z above.

Table A-17b: This subtable provides 23 compounds A-17b.01 to A-17b.23 of formula (Ia-A-17) wherein R¹ is 3-(trifluoromethyl)phenyl, R⁸ is hydrogen and substituents NH-L¹-G are as defined in Table Z above.

Table A-17c: This subtable provides 23 compounds A-17c.01 to A-17c.23 of formula (Ia-A-17) wherein R¹ is 3-cyclopropylphenyl, R⁸ is hydrogen and substituents NH-L¹-G are as defined in Table Z above.

Also made available are certain intermediate compounds of formulae (II), (V), (VI) and (XVIII), some of which are novel. For example:
A compound of formula (II), wherein R¹ and A are as defined in any one of Tables A-1 to A-17, and their subtables:

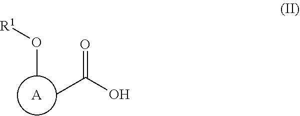

and more specifically, compounds of formula (II-A-1i) to (II-A-17i), as shown in the Table II below, wherein R¹, R⁷, R⁸ and R⁹ are as defined in any one of Tables A-1 to A-17, and their respective subtables:

TABLE II

| | |
|---|---|
| 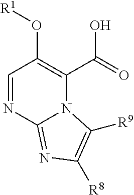 | II-A-1i |
| 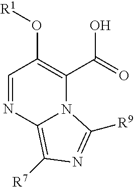 | II-A-2i |
| 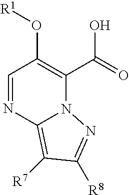 | II-A-3i |
| 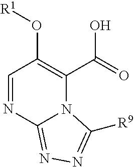 | II-A-4i |
| 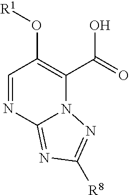 | II-A-5i |
| 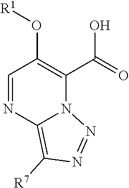 | II-A-6i |
| 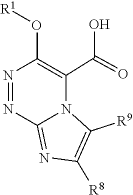 | II-A-7i |
| 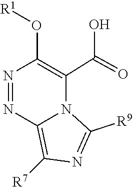 | II-A-8i |
| 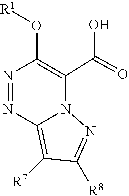 | II-A-9i |
| 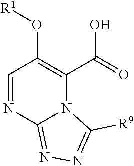 | II-A-10i |
| 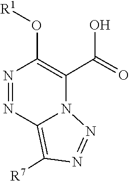 | II-A-11i |
| 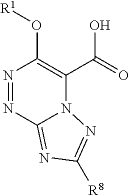 | II-A-12i |
| 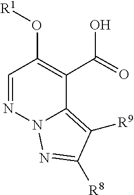 | II-A-13i |
| 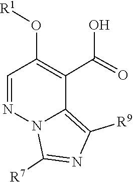 | II-A-14i |
| 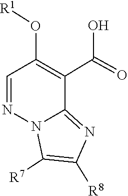 | II-A-15i |
| 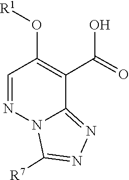 | II-A-16i |
| 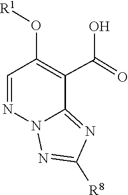 | II-A-17i |

A compound of formula (V), wherein X² is a suitable leaving group such as fluoro, chloro, bromo, iodo, BF₃K, B(OH)₂ or B(pinacol), and wherein A is as defined in any one of Tables A-1 to A-17, and their respective subtables, and NH-L¹-G is as defined in Table Z:

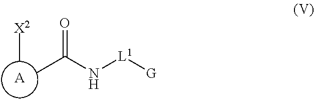

and more specifically, compounds of formula (V-A-1i) to (V-A-17i), as shown in the Table V below, wherein X² is a suitable leaving group such as fluoro, chloro, bromo, iodo, BF3K, B(OH)₂ or B(pinacol), and wherein R⁷, R⁸ and R⁹ are as defined in any one of Tables A-1 to A-17, and their respective subtables, and NH-L¹-G is as defined in Table Z:

TABLE V

| | |
|---|---|
| 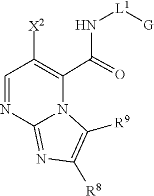 | V-A-1i |
| 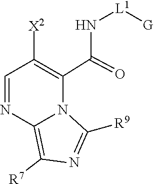 | V-A-2i |
| 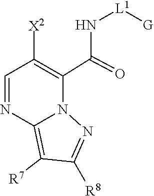 | V-A-3i |
| 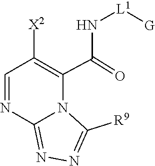 | V-A-4i |
| 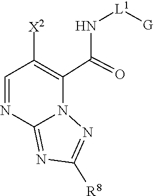 | V-A-5i |
| 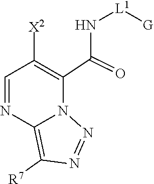 | V-A-6i |
| 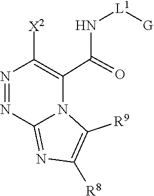 | V-A-7i |
| 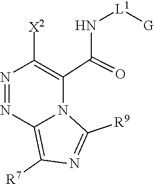 | V-A-8i |
| 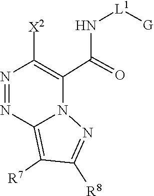 | V-A-9i |
| 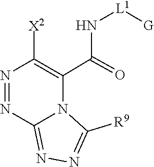 | V-A-10i |
| 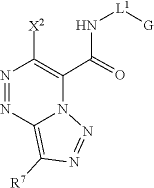 | V-A-11i |
| 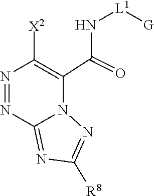 | V-A-12i |
| 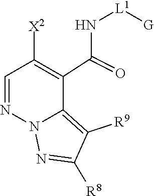 | V-A-13i |
| 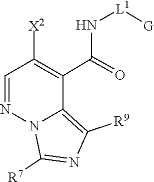 | V-A-14i |
| 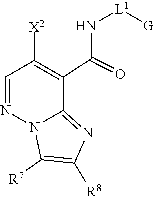 | V-A-15i |
| 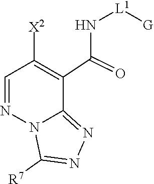 | V-A-16i |
| 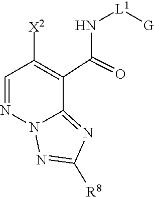 | V-A-17i |

A compound of formula (VI), wherein X¹ is C₁-C₄-alkoxy and X² is a suitable leaving group such as fluoro, chloro, bromo, iodo, BF₃K, B(OH)₂ or B(pinacol), and wherein A is as defined in any one of Tables A-1 to A-17, and their respective subtables:

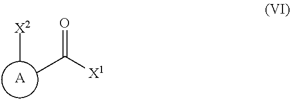

and more specifically, compounds of formula (VI-A-1i) to (VI-A-17i), as shown in the Table VI below, wherein X¹ is C₁-C₄-alkoxy, such as methoxy or ethoxy, and X² is a suitable leaving group such as fluoro, chloro, bromo, iodo, BF3K, B(OH)₂ or B(pinacol), and wherein R⁷, R⁸ and R⁹ are as defined in any one of Tables A-1 to A-17, and their respective subtables:

TABLE VI

| | |
|---|---|
| 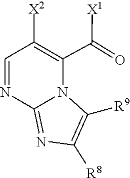 | VI-A-1i |
| 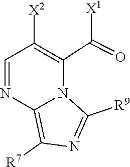 | VI-A-2i |
| 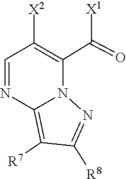 | VI-A-3i |
| 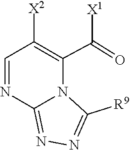 | VI-A-4i |
| 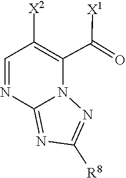 | VI-A-5i |
| 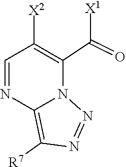 | VI-A-6i |
| 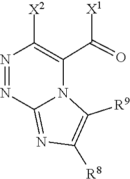 | VI-A-7i |
| 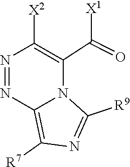 | VI-A-8i |
| 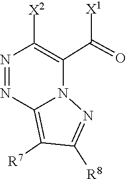 | VI-A-9i |
| 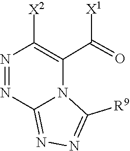 | VI-A-10i |
| 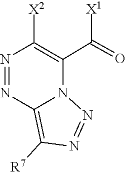 | VI-A-11i |
| 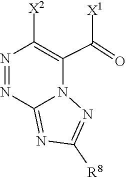 | VI-A-12i |
| 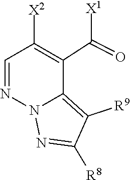 | VI-A-13i |
| 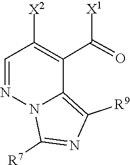 | VI-A-14i |
| 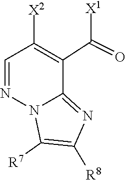 | VI-A-15i |
| 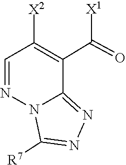 | VI-A-16i |
| 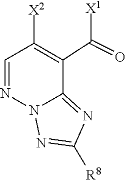 | VI-A-17i |

A compound of formula (XVIII), wherein X⁶ is chloro, bromo, iodo, trifluoromethanesulfonyl-O—, and wherein R¹ and A are as defined in any one of Tables A-1 to A-17, and their subtables:

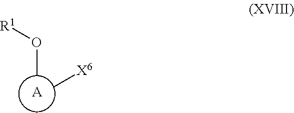

and more specifically, compounds of formula (XVIII-A-1i) to (XVIII-A-17i), as shown in the Table XVIII below, wherein X⁶ is chloro, bromo, iodo, trifluoromethanesulfonyl-O—, and wherein R¹, R⁷, R⁸ and R⁹ are as defined in any one of Tables A-1 to A-17, and their respective subtables:

TABLE XVIII

| | |
|---|---|
| 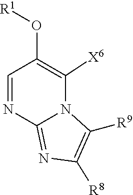 | XVIII-A-1i |
| 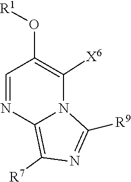 | XVIII-A-2i |
| 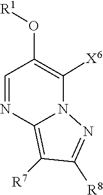 | XVIII-A-3i |
| 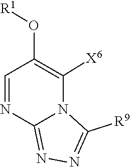 | XVIII-A-4i |
| 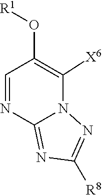 | XVIII-A-5i |
| 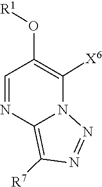 | XVIII-A-6i |
| 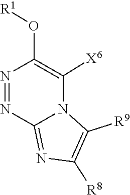 | XVIII-A-7i |
| 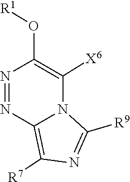 | XVIII-A-8i |
| 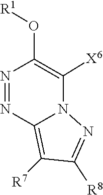 | XVIII-A-9i |
| 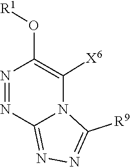 | XVIII-A-10i |
| 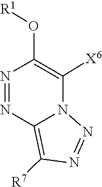 | XVIII-A-11i |
| 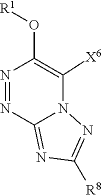 | XVIII-A-12i |
| 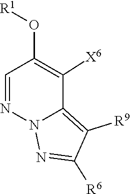 | XVIII-A-13i |
| 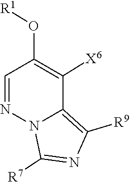 | XVIII-A-14i |
| 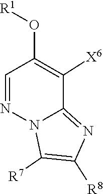 | XVIII-A-15i |
| 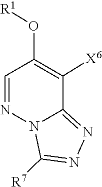 | XVIII-A-16i |
| 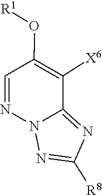 | XVIII-A-17i |

In further aspect, the present invention accordingly makes available compounds of formulae (II), (V), (VI), and (XVIII) wherein in each case, as applicable, R¹, R², R³, R⁴, R⁵, L¹, G, and A, including R⁷, R⁸, and R⁹, are as defined for formula (I) in the first aspect; and X² is a suitable leaving group such as fluoro, chloro, bromo, iodo, BF₃K, B(OH)₂ or B(pinacol); X³ is OH or C₁-C₄-alkoxy; and X⁶ is chloro, bromo, iodo, trifluoromethanesulfonyl-O—. Furthermore, the corresponding embodiments illustrated for formula (I) also apply to the compounds of formulae (II), (V), (VI), and (XVIII).

### EXAMPLES

The Examples which follow serve to illustrate the invention and are not meant in any way to limit the invention.

The compounds of the invention can be distinguished from known compounds by virtue of greater efficacy at low application rates, which can be verified by a person skilled in the art using the experimental procedures outlined in the Examples, using lower application rates, if necessary, for example 60 ppm, 20 ppm or 2 ppm.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius (° C.) and “m.p.” means melting point. LC/MS or LC-MS or LCMS means Liquid Chromatography Mass Spectroscopy and the description of the apparatus, and the methods is as follows.

¹H NMR and ¹⁹F NMR measurements were recorded on a Bruker 400 MHz spectrometer, chemical shifts are given in ppm relevant to a TMS (¹H) and CFCl₃ (¹⁹F) standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

### Method A:

Spectra were recorded on a Mass Spectrometer 6410 Triple Quadruple Mass Spectrometer from Agilent Technologies equipped with an electrospray source (Positive and Negative Polarity Switch, Capillary (kV) 4.00, Scan Type MS2 Scan, Fragmentor (V) 100.00, Gas Temperature (° C.) 350, Gas Flow (L/min) 11, Nebulizer Gas (psi) 45, Mass range: 110 to 1000 Da) and an Agilent 1200 Series HPLC: DAD Wavelength range: 210 to 400 nm, Column: KINETEX EVO C₁₈, Column length: 50 mm, Internal diameter of column: 4.6 mm, Particle Size: 2.6 μm, Column oven temperature: 40° C.

### Gradient Conditions:

Solvent A: Water with 0.1% formic acid:Acetonitrile: 95:5 v/v
Solvent B: Acetonitrile with 0.1% formic acid

| Time | | | Flow rate |
|---|---|---|---|
| (minutes) | A (%) | B (%) | (ml/min) |
| 0 | 90 | 10 | 1.8 |
| 0.9 | 0 | 100 | 1.8 |
| 1.8 | 0 | 100 | 1.8 |
| 2.2 | 90 | 10 | 1.8 |

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Method B:

Spectra were recorded on a Mass Spectrometer 6410 Triple Quadruple Mass Spectrometer from Agilent Technologies equipped with an electrospray source (Positive and Negative Polarity Switch, Capillary (kV) 7.00, Scan Type MS2 Scan, Fragmentor (V) 120.00, Gas Temperature (° C.) 350, Gas Flow (L/min) 11, Nebulizer Gas (psi) 40, Mass range: 110 to 650 Da) and an Agilent 1200 Series HPLC: DAD Wavelength: 254 nm, Column: KINETEX EVO C₁₈, Column length: 50 mm, Internal diameter of column: 4.6 mm, Particle Size: 2.6 μm, Column oven temperature: 40° C.

### Gradient Conditions:

Solvent A: Water with 0.1% formic acid:Acetonitrile:95:5 v/v
Solvent B: Acetonitrile with 0.1% formic acid

| Time | | | Flow rate |
|---|---|---|---|
| (minutes) | A (%) | B (%) | (ml/min) |
| 0 | 90 | 10 | 1.8 |
| 0.9 | 0 | 100 | 1.8 |
| 1.8 | 0 | 100 | 1.8 |
| 2.2 | 90 | 10 | 1.8 |
| 2.5 | 90 | 10 | 1.8 |

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Method C:

Spectra were recorded on a Mass Spectrometer Acquity QDA Mass Spectrometer from Waters equipped with an electrospray source (Positive and Negative Polarity Switch, Capillary (kV) 0.8, Cone Voltage (V) 25.00, Full Scan, Source Temperature (° C.) 120, Desolvatation Gas Flow (L/Hr) 1000, Desolvation Temperature (° C.) 600, Gas Flow® Cone (L/Hr) 50, Mass range: 110 to 850 Da) and HPLC: DAD Wavelength range: 230 to 400 nm, a column Acquity UPLC HSS T3 C18 Column length: 30 mm, Internal diameter of column: 2.1 mm, Particle Size: 1.8 μm, Column oven temperature: 40° C.

### Gradient Conditions:

Solvent A: Water with 0.1% formic acid:Acetonitrile: 95:5 v/v
Solvent B: Acetonitrile with 0.05% formic acid

| Time | | | Flow rate |
|---|---|---|---|
| (minutes) | A (%) | B (%) | (ml/min) |
| 0 | 90 | 10 | 0.6 |
| 0.2 | 90 | 10 | 0.6 |
| 0.3 | 50 | 50 | 0.6 |
| 0.6 | 0 | 100 | 0.6 |
| 1.3 | 0 | 100 | 0.6 |
| 1.4 | 90 | 10 | 0.6 |
| 1.6 | 90 | 10 | 0.6 |

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Method D:

Spectra were recorded on a Mass Spectrometer Acquity SDQ Mass Spectrometer from Waters equipped with an electrospray source (Positive and Negative Polarity Switch, Capillary (kV) 3.0, Full Scan, Cone voltage (V) 41.0, Source Temperature (° C.) 150, Desolvation Temperature 500° C., Gas Flow® Cone (L/Hr) 50, Mass range: 110 to 800 Da) and HPLC ‘H’ class: DAD Wavelength range: 210 to 400 nm, a column Acquity UPLC HSS T3 C18 Column length: 30 mm, Internal diameter of column: 2.1 mm, Particle Size: 1.8 μm, Column oven temperature: 40° C.

### Gradient Conditions:

Solvent A: Water with 0.1% formic acid:Acetonitrile: 95:5 v/v
Solvent B: Acetonitrile with 0.05% formic acid

| Time | | | Flow rate |
|---|---|---|---|
| (minutes) | A (% | B (%) | (ml/min) |
| 0 | 90 | 10 | 0.6 |
| 0.2 | 90 | 10 | 0.6 |
| 0.3 | 50 | 50 | 0.6 |
| 0.6 | 0 | 100 | 0.6 |
| 1.3 | 0 | 100 | 0.6 |
| 1.4 | 90 | 10 | 0.6 |
| 1.6 | 90 | 10 | 0.6 |

### Method E:

Spectra were recorded on a Mass Spectrometer from Waters (QDa) (Polarity: positive and negative ions), Detector Gain 1, Temperature Probe: 500° C., Cone Voltage: 10V, ESI Capillary Positive Voltage 0.8—Negative Voltage 0.8, Sampling Frequency 5 Hz, Mass range: 100 to 850 Da.

Chiral column (analytical)SFC:Waters Acquity UPC²/QDa PDA Detector Waters Acquity UPC² Column: Daicel SFC CHIRALPAK® AY, 3 m, 0.3 cm×10 cm, 40° C. Mobile phase: A: CO₂ B: IPA isocratic: 25% B ABPR: 1800 psi Flow rate: 2.0 ml/min Detection: 240 nm Sample concentration: 1 mg/mL in MeOH/ACN Injection: 2 μL

### Formulation Examples

The Examples which follow serve to illustrate the invention.

| Wettable powders | a) | b) | c) |
|---|---|---|---|
| active ingredient [compound of formula (i)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyvinyl glycol ether (7-8 mol | — | 2% | — |
| of vinyl oxide) | | | |
| highly dispersed silicic acid | 5% | 10% | 10% |
| kaolin | 62% | 27% | — |

The active ingredient is thoroughly mixed with e adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (i)] | 25% | 50% | 75% |
| light mineral oil | 5% | 5% | 5% |
| highly dispersed silicic acid | 5% | 5% | — |
| kaolin | 65% | 40% | — |
| talcum | — | | 20% |

The active ingredient is thoroughly mixed with the adjuvants and the mixture is thoroughly ground in a suitable mill, affording powders that can be used directly for seed treatment.

Emulsifiable concentrate

| | |
|---|---|
| active ingredient [compound of formula (i)] | 10% |
| octylphenol polyvinyl glycol ether (4-5 mol of vinyl oxide) | 3% |
| calcium dodecylbenzenesulfonate | 3% |
| castor oil polyglycol ether (35 mol of vinyl oxide) | 4% |
| cyclohexanone | 30% |
| xylene mixture | 50% |

Emulsions of any require dilution, which can be use in plant protection, can be obtained from this concentrate by dilution with water.

| Dusts | a) | b) | c) |
|---|---|---|---|
| active ingredient [compound of formula (i)] | 5% | 6% | 4% |
| talcum | 95% | — | — |
| kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

Extruded granules

| | |
|---|---|
| active ingredient [compound of formula (i)] | 15% |
| sodium lignosulfonate | 2% |
| carboxymethylcellulose | 1% |
| kaolin | 82% |

The active ingredient is mixed an ground with the adjuvants, and the mixture is moistened with water. The mixture is extruded and then dried in a stream of air.

Coated granules

| | |
|---|---|
| active ingredient [compound of formula (i)] | 8% |
| polyvinyl glycol (mol. wt. 200) | 3% |
| kaolin | 89% |

The finely ground active ingredient is uniformly applied, in a mixer, to kaolin moistened with polyvinyl glycol. Non-dusty coated granules are obtained in this manner.

Suspension concentrate

| | |
|---|---|
| active ingredient [compound of formula (i)] | 40% |
| propylene glycol | 10% |
| nonylphenol polyvinyl glycol ether (15 mol of vinyl oxide) | 6% |
| sodium lignosulfonate | 10% |
| carboxymethylcellulose | 1% |
| silicone oil (in the form of a 75% emulsion in water) | 1% |
| water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of | 0.5% |
| a 20% solution in water) | |
| monoazo-pigment calcium salt | 5% |
| silicone oil (in the form of a 75% emulsion in water) | 0.2% |
| water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow-Release Capsule Suspension

28 parts of a combination of the compound of formula (I) are mixed with 2 parts of an aromatic solvent and 7 parts of toluene diisocyanate/polymvinyl-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### LIST OF ABBREVIATIONS

Abbreviations used in synthesis schemes and preparatory examples
ACN acetonitrile
Boc t-butoxycarbonyl
DBU 1,8-diazabicyclo[5.4.0]undec-7-ene
DCM dichloromethane
DDQ 2,3-dichloro-5,6-dicyano-1,4-benzoquinone
DMSO dimethyl sulfoxide
DMSO-d⁶ deuterated dimethylsulfoxide
DPEN diphenylvinyldiamine
Et3N triethylamine
EtOAc ethyl acetate
hr/hrs hour/hours
MeCN acetonitrile
MeOH ethanol
Ms methanesulfonyl (mesyl)
n-Bu n-butyl
NHC N-heterocyclic carbene
NPhth phthalimide-1-yl
OMs mesylate group
OTf triflate group
OTs tosylate group
PdCl2dppf 1,1′-bis(diphenylphosphino)ferrocene]palladium(II) dichloride
TBME tert-butyl methyl ether
TEA triethylamine
TEMPO (2,2,6,6-tetramethylpiperidin-1-yl)oxidanyl
Tf trifluoromethanesulfonyl (triflyl)
TFA trifluoroacetic acid
THE tetrahydrofuran
Ts p-toluenesulfonyl (tosyl)
X-Phos 2-dicyclohexylphosphino-2′,4′,6′-triisopropylbiphenyl
aq. aqueous
° C. degrees Celsius
equiv. equivalent
h hour(s)
LC/MS or LC-MS liquid chromatography mass spectrometry
M molar
MHz megahertz
min minutes
mp or MP melting point
NMR nuclear magnetic resonance
ppm parts per million
RT or rt room temperature
Rt retention time
RBF round-bottom flask

### Preparation Examples

The following examples further illustrate, but do not limit, the invention. Those skilled in the art will promptly recognize appropriate variations from the procedures both as to reactants and as to reaction conditions and techniques.

Unless indicated otherwise, ¹H NMR spectra are recorded at 400 MHz and ¹⁹F NMR spectra are recorded at 377 MHz, and chemical shifts are recorded in ppm. The following abbreviations are used: s=singlet; br s=broad singlet; d=doublet; br d=broad doublet; dd=double doublet; dt=double triplet; t=triplet, tt=triple triplet, q=quartet, quin=quintuplet, sept=septet; m=multiplet.

Throughout this description, temperatures are given in degrees Celsius (° C.). “MP” means melting point. “Rt” means retention time. LC/MS means Liquid Chromatography Mass Spectrometry. LC/MS apparatus and methods are: Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Example P1: This Example Illustrates the Preparation of 6-(3-cyclopropylphenoxy)-N-[2-(2,4-dichlorophenyl)-2-fluoro-ethyl]imidazo[1,2-a]pyrimidine-5-carboxamide (Compound 1.1 of Table T1)

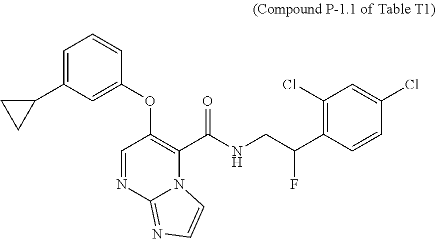 [SMILES: O=C(NCC(F)c1ccc(Cl)cc1Cl)c1c(Oc2cccc(C3CC3)c2)cnc2nccn12]

### a) Preparation of ethyl 2-(3-cyclopropylphenoxy)acetate

In a single-neck round-bottom flask, caesium carbonate (5.82 g, 17.8 mmol) was added to a solution of 3-cyclopropylphenol (2.0 g, 14.9 mmol) in acetonitrile (22 mL). To this, ethyl 2-bromoacetate (2.98 g, 17.8 mmol) was added and the resulting reaction mixture was stirred at rt for 4 hr. The progress of the reaction was monitored by LCMS. The reaction mixture was then diluted with water and extracted with EtOAc. The organic layer was washed with brine, dried over sodium sulphate and concentrated under reduced pressure. The resulting crude residue was purified by silica gel chromatography (cyclohexane/EtOAc) to afford ethyl 2-(3-cyclopropylphenoxy)acetate.

LCMS (Method B): retention time 1.48 min, 221 (M+H)

### b) Preparation of ethyl 2-(3-cyclopropylphenoxy)-3-(dimethylamino)prop-2-enoate

In a sealed glass reactor, ethyl 2-(3-cyclopropylphenoxy)acetate (1.5 g, 6.8 mmol) and 1-tert-butoxy-N,N,N′,N′tetramethyl-methanediamine (9.2 g, 48.0 mmol) solution were heated to 90° C. for 2 hr. The progress of the reaction was monitored by LCMS. The reaction mixture was then diluted with water and extracted with EtOAc. The organic layer was washed with brine, dried over sodium sulphate and concentrated under reduced pressure to obtain a crude residue. The resulting crude residue was purified by silica gel chromatography (cyclohexane/EtOAc) to afford ethyl 2-(3-cyclopropylphenoxy)-3-(dimethylamino)prop-2-enoate.

LCMS (Method B): retention time 1.52 min, 276 (M+H)

### c) Preparation of 6-(3-cyclopropylphenoxy)-8H-imidazo[1,2-a]pyrimidin-5-one

In a round-bottom flask, the mixture of ethyl 2-(3-cyclopropylphenoxy)-3-(dimethylamino)prop-2-enoate (1.0 g, 3.63 mmol), 2H-imidazol-2-amine (0.30 g, 3.63 mmol) and sodium acetate (0.30 g, 3.63 mmol) in acetic acid (10 mL) was stirred at 110° C. for 4 hr. The progress of the reaction was monitored by LCMS.

The reaction mixture was then concentrated under reduced pressure followed by two successive co-distillations with toluene under reduced pressure to obtain a crude residue. The resulting crude residue was purified by silica gel chromatography (cyclohexane/EtOAc) to afford 6-(3-cyclopropylphenoxy)-8H-imidazo[1,2-a]pyrimidin-5-one as a brown solid.

LCMS (Method B): retention time 1.24 min, 268 (M+H)

### d) Preparation of 5-chloro-6-(3-cyclopropylphenoxy)imidazo[1,2-a]pyrimidine

In a round-bottom flask, the mixture of 6-(3-cyclopropylphenoxy)-8H-imidazo[1,2-a]pyrimidin-5-one (0.40 g, 1.49 mmol), N,N-dimethylaniline (0.78 g, 6.13 mmol) and phosphorus(V) oxychloride (10.6 mL, 113 mmol) was stirred at 90° C. for 12 hr. The progress of the reaction was monitored by LCMS. After the completion, the reaction mixture was diluted with ice water and extracted with EtOAc. The organic layer was washed with brine, dried over sodium sulphate and concentrated under reduced pressure to obtain a crude residue. The resulting crude residue was purified by silica gel chromatography (cyclohexane/EtOAc) to afford 5-chloro-6-(3-cyclopropylphenoxy)imidazo[1,2-a]pyrimidine.

LCMS (Method B): retention time 1.42 min, 286 (M+H)

### e) Preparation of methyl 6-(3-cyclopropylphenoxy)imidazo[1,2-a]pyrimidine-5-carboxylate

An autoclave vessel was charged with 5-chloro-6-(3-cyclopropylphenoxy)imidazo[1,2-a]pyrimidine (0.08 g, 0.28 mmol), triethylamine (0.08 mL, 0.56 mmol), Pd(dppf)Cl₂CH₂Cl₂ (0.057 g, 0.07 mmol) and methanol (20 mL). The reactor was flushed three times with carbon monoxide gas and loaded with 10 bar of carbon monoxide. The reaction mixture was heated to 80° C. for 1.5 hr. The progress of the reaction was monitored by LCMS. The reaction mixture was then diluted with water and extracted with EtOAc. The organic layer was washed with brine, dried over sodium sulphate and concentrated under reduced pressure to obtain a crude residue. The resulting crude residue was purified by silica gel chromatography (cyclohexane/EtOAc) to afford methyl 6-(3-cyclopropylphenoxy)imidazo[1,2-a]pyrimidine-5-carboxylate as a yellow solid.

LCMS (Method B): retention time 1.36 min, 310 (M+H)

### f) Preparation of lithium 6-(3-cyclopropylphenoxy)imidazo[1,2-a]pyrimidine-5-carboxylate

To a solution of methyl 6-(3-cyclopropylphenoxy)imidazo[1,2-a]pyrimidine-5-carboxylate (0.10 g, 0.32 mmol) in tetrahydrofuran (0.45 mL) and water (0.22 mL) was added lithium hydroxide monohydrate (0.041 g, 0.96 mmol). The reaction was allowed to proceed at rt for 2 hr. The progress of the reaction was monitored by TLC and LCMS. The reaction mixture was then concentrated under reduced pressure at 30° C. followed by two successive co-distillations with toluene to afford lithium 6-(3-cyclopropylphenoxy)imidazo[1,2-a]pyrimidine-5-carboxylate which was used as such in the next step.

LCMS (Method B): retention time 0.45 min, 296 (M+H)

### g) Preparation of 6-(3-cyclopropylphenoxy)-N-[2-(2,4-dichlorophenyl)-2-fluoro-ethyl]imidazo[1,2-a]pyrimidine-5-carboxamide (Compound P-1.1, Table T1)

To lithium 6-(3-cyclopropylphenoxy)imidazo[1,2-a]pyrimidine-5-carboxylate (0.10 g, 0.27 mmol) in EtOAc (1.35 mL), 2-(2,4-dichlorophenyl)-2-fluoro-ethanamine (0.067 g, 0.32 mmol) was added 1-propanephosphonic anhydride solution (T3P, 50% in EtOAc, 0.48 mL, 0.81 mmol) and triethylamine (0.08 mL, 0.59 mmol) at 0° C. This reaction mixture was stirred at rt for 2 hr. The progress of the reaction was monitored by TLC and LCMS. After completion, the reaction mixture was diluted with water and extracted with EtOAc. The organic layer was washed with brine, dried over sodium sulfate and concentrated under reduced pressure. The resulting crude residue was purified by silica gel chromatography (cyclohexane/EtOAc) to afford 6-(3-cyclopropylphenoxy)-N-[2-(2,4-dichlorophenyl)-2-fluoro-ethyl]imidazo[1,2-a]pyrimidine-5-carboxamide as a yellow solid.

¹H NMR (400 MHz, CDCl₃) δ ppm 8.79 (d, J=1.38 Hz, 1H), 8.36 (s, 1H), 7.95 (d, J=1.38 Hz, 2H), 7.36-7.38 (m, 1H), 7.29-7.34 (m, 1H), 7.19-7.25 (m, 1H), 7.07-7.12 (m, 1H), 6.94-6.99 (m, 1H), 6.79-6.84 (m, 1H), 6.73-6.77 (m, 1H), 5.85-6.00 (m, 1H), 4.06-4.21 (m, 1H), 3.83-3.98 (m, 1H), 1.88-1.95 (m, 1H), 1.00-1.08 (m, 2H), 0.70-0.75 (m, 2H).

LCMS (Method B): retention time 1.55 min, 485 (M+H)

### Example P2: This Example Illustrates the Preparation of 6-(3-cyclopropylphenoxy)-N-[2-(2,4-dichlorophenyl)-2-fluoro-ethyl]-[1,2,4]triazolo[1,5-a]pyrimidine-7-carboxamide (Compound P-1.2 of Table T1)

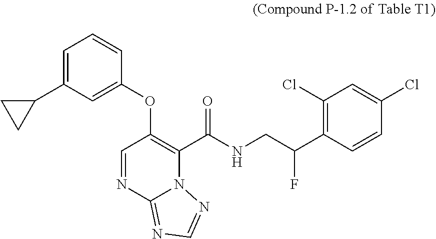 [SMILES: O=C(NCC(F)c1ccc(Cl)cc1Cl)c1c(Oc2cccc(C3CC3)c2)cnc2ncnn12]

Ethyl 2-(3-cyclopropylphenoxy)-3-(dimethylamino)prop-2-enoate was prepared as described in example 1, steps a) and b).

### a) Preparation of 6-(3-cyclopropylphenoxy)-4H-[1,2,4]triazolo[1,5-a]pyrimidin-7-one

To a round-bottom flask equipped with a nitrogen balloon, ethyl 2-(3-cyclopropylphenoxy)-3-(dimethylamino)prop-2-enoate (3 g, 10.35 mmol) and 4H-1,2,4-triazol-3-amine (0.96 g, 10.35 mmol) were dissolved in acetic acid (30 mL). To that, sodium acetate (0.86 g, 10.35 mmol) was added and stirred at 90° C. for 18 hr under nitrogen. The progress was monitored by TLC and LCMS analysis. After full consumption of the starting material, the reaction mixture was cooled to room temperature, diluted with ice-cold water extracted with EtOAc. The combined organic layers were dried over sodium sulfate and concentrated under reduced pressure. The resulting crude residue was purified by silica gel chromatography (0-30% EtOAc in cyclohexane) to afford 6-(3-cyclopropylphenoxy)-4H-[1,2,4]triazolo[1,5-a]pyrimidin-7-one as a white solid.

¹H NMR (400 MHz, DMSO-d6) δ ppm 8.29-8.34 (m, 2H) 7.15 (t, J=7.86 Hz, 1H) 6.78 (s, 1H) 6.78 (d, J=7.70 Hz, 2H) 6.72 (d, J=7.39 Hz, 1H) 1.89 (s, 1H) 0.88-0.96 (m, 2H) 0.59-0.72 (m, 2H) LCMS (Method B): retention time 1.21 min, 269 (M+H)

### b) Preparation of 7-chloro-6-(3-cycloproylphenoxy)-[1.2.4]triazolo[1,5-a]pyrimidine

To a round-bottom flask equipped with a nitrogen balloon and a condenser, 6-(3-cyclopropylphenoxy)-4H-[1,2,4]triazolo[1,5-a]pyrimidin-7-one (0.95 g, 3.38 mmol) was added to phosphorus oxychloride (24 mL, 257 mmol). To that, N,N-dimethylaniline (1.77 g, 13.88 mmol) was added and the resulting reaction mixture was stirred at 85° C. for 12 hr. The progress of the reaction was monitored by TLC and LCMS analysis.

After completion, the reaction mixture was cooled down to rt and added at once to crushed ice and extracted with EtOAc. The combined organic layers were dried over sodium sulfate and concentrated in vacuo to obtain a crude residue which was then purified by silica gel chromatography (0-20% EtOAc in cyclohexane) to afford 7-chloro-6-(3-cyclopropylphenoxy)-[1,2,4]triazolo[1,5-a]pyrimidine as a pale yellow solid.

¹H NMR (400 MHz, CDCl₃) δ ppm 8.70 (s, 1H), 8.59 (d, J=1.50 Hz, 1H), 7.22-7.29 (m, 1H), 6.88 (d, J=7.75 Hz, 1H), 6.70-6.77 (m, 2H), 1.82-1.94 (m, 1H), 0.93-1.06 (m, 2H), 0.64-0.76 (m, 2H).

LCMS (Method B): retention time 1.43 min, 287 (M+H)

### c) Preparation of methyl 6-(3-cyclopropylphenoxy)-[1,2,4]triazolo[1,5-a]pyrimidine-7-carboxylate

An autoclave vessel was charged with 7-chloro-6-(3-cyclopropylphenoxy)-[1,2,4]triazolo[1,5-a]pyrimidine (0.85 g, 2.82 mmol), triethylamine (0.57 g, 5.63 mmol), Pd(dppf)Cl₂CH₂Cl₂ (0.58 g, 0.70 mmol) and methanol (30 mL). The reactor was flushed three times with carbon monoxide gas and loaded with 10 bar of carbon monoxide. The reaction mixture was heated to 80° C. for 1.5 hr. The progress of the reaction was monitored by LCMS. After completion, the reaction mixture was cooled down to rt and filtered through celite. The filtrate was then evaporated under reduced pressure. The crude compound was purified by silica gel chromatography (0-20% EtOAc in cyclohexane) to afford methyl 6-(3-cyclopropylphenoxy)-[1,2,4]triazolo[1,5-a]pyrimidine-7-carboxylate as a pale yellow gummy mass.

¹H NMR (400 MHz, CDCl₃) δ ppm 8.65 (s, 1H), 8.48 (s, 1H), 7.14-7.22 (m, 1H), 6.83 (d, J=7.75 Hz, 1H), 6.67-6.75 (m, 2H), 3.96 (s, 3H), 1.77-1.85 (m, 1H), 0.83-1.01 (m, 2H), 0.59-0.65 (m, 2H).

LCMS (Method B): retention time 1.4 min, 311 (M+H)

### d) Preparation of 6-(3-cyclopropylphenoxy)-[1,2,4]triazolo[1,5-a]pyrimidine-7-carboxylic acid

To a solution of methyl 6-(3-cyclopropylphenoxy)-[1,2,4]triazolo[1,5-a]pyrimidine-7-carboxylate (0.30 g, 0.93 mmol) in tetrahydrofuran (1.3 mL) and water (0.6 mL) was added lithium hydroxide (0.12 g, 2.80 mmol). The reaction mixture was stirred at 10° C. for 1 hour. The progress of the reaction was monitored by TLC and LCMS. After completion of the reaction, the reaction mixture was diluted with water and washed with EtOAc. The aqueous layer was then acidified with 2N HCl at 0° C. and extracted with EtOAc. The combined organic layers were dried over sodium sulfate and concentrated under reduced pressure to obtain 2-(3-cyclopropylphenoxy)imidazo[1,5-b]pyridazine-3-carboxylic acid as a beige solid.

LCMS (Method B): retention time 1.12 min, 297 (M+H)

### e) Preparation of 6-(3-cyclopropylphenoxy)-N-[2-(2,4-dichlorophenyl)-2-fluoro-ethyl]-[1,2,4]triazolo[1,5-a]pyrimidine-7-carboxamide (Compound P-1.2, Table T

To 6-(3-cyclopropylphenoxy)-[1,2,4]triazolo[1,5-a]pyrimidine-7-carboxylic acid (0.3 g, 0.91 mmol) in EtOAc (4.5 mL), 2-(2,4-dichlorophenyl)-2-fluoro-ethanamine (0.23 g, 1.1 mmol) was added, followed by the addition of 1-propanephosphonic anhydride solution (T3P, 50% in EtOAc, 1.74 g, 2.73 mmol) and triethylamine (0.58 g, 5.47 mmol). The reaction mixture was stirred at rt for 18 hr. The reaction progress was monitored by TLC and LCMS. After completion, the reaction mixture was diluted with water, extracted with EtOAc and the organic layer was washed with brine, dried over sodium sulfate and concentrated under reduced pressure. The resulting crude residue was purified by silica gel chromatography (0-30% EtOAc in cyclohexane) to afford 6-(3-cyclopropylphenoxy)-N-[2-(2,4-dichlorophenyl)-2-fluoro-ethyl]-[1,2,4]triazolo[1,5-a]pyrimidine-7-carboxamide as a white solid.

¹H NMR (400 MHz, CDCl₃) δ ppm 8.99 (br s, 1H), 8.74 (s, 1H), 8.58 (s, 1H), 7.46 (br d, J=8.44 Hz, 1H), 7.41 (s, 1H), 7.26 (br t, J=8.50 Hz, 2H), 6.90 (br d, J=7.46 Hz, 1H), 6.75-6.81 (m, 2H), 6.07-5.94 (m, 1H), 4.12-4.29 (m, 1H), 3.76-3.91 (m, 1H), 1.83-1.93 (m, 1H), 1.00 (m, 2H), 0.71 (m, 2H).

LCMS (Method C): retention time 1.22 min, 486.2 (M+H)

### Example P3: This Example Illustrates the Preparation of 6-(3-cyclopropylphenoxy)-N-[2-(2,4-dichlorophenyl)-2-fluoro-ethyl]pyrazolo[1,5-a]pyrimidine-7-carboxamide (Compound P-1.3 of Table T1)

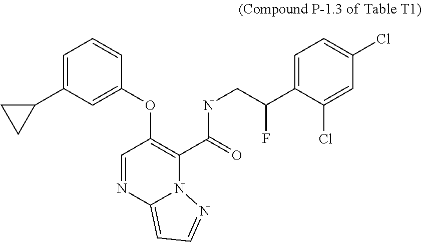 [SMILES: O=C(NCC(F)c1ccc(Cl)cc1Cl)c1c(Oc2cccc(C3CC3)c2)cnc2ccnn12]

Ethyl 2-(3-cyclopropylphenoxy)-3-(dimethylamino)prop-2-enoate was prepared as described in example 1, steps a) and b).

### a) Preparation of 6-(3-cyclopropylphenoxy)-4H-pyrazolo[1,5-a]pyrimidin-7-one

In a single-neck round-bottom flask, a mixture of ethyl 2-(3-cyclopropylphenoxy)-3-(dimethylamino)prop-2-enoate (1.0 g, 3.63 mmol), 3H-pyrazol-3-amine (0.30 g, 3.63 mmol) and sodium acetate (0.30 g, 3.63 mmol) in acetic acid (2 mL) was stirred at 90° C. for 24 hr. The progress of the reaction was monitored by LCMS. The reaction mixture was then concentrated under reduced pressure, A crude residue was obtained after two successive co-distillations with toluene under reduced pressure. The resulting residue was eventually purified by silica gel chromatography (cyclohexane/EtOAc) to afford 6-(3-cyclopropylphenoxy)-4H-pyrazolo[1,5-a]pyrimidin-7-one as a brown solid.

LCMS (Method B): retention time 1.24 min, 268 (M+H)

### b) Preparation of 7-chloro-6-(3-cyclopropylphenoxy)pyrazolo[1,5-a]pyrimidine

In a round-bottom flask, to a mixture of 6-(3-cyclopropylphenoxy)-4H-pyrazolo[1,5-a]pyrimidin-7-one (0.33 g, 1.23 mmol) and phosphorus(V) oxychloride (8.76 mL 93.83 mmol), N,N-dimethylaniline (0.64 g, 5.06 mmol) was added at 0° C. The reaction mixture was heated at 90° C. for 12 hr. The progress of the reaction was monitored by LCMS. The reaction mixture was then diluted with ice water and extracted with EtOAc. The organic layer was washed with brine, dried over sodium sulphate and concentrated under reduced pressure. The resulting crude residue was purified by silica gel chromatography (cyclohexane/EtOAc) to afford 7-chloro-6-(3-cyclopropylphenoxy)pyrazolo[1,5-a]pyrimidine.

LCMS (Method B): retention time 2.53 min, 286 (M+H)

### c) Preparation of methyl 6-(3-cyclopropylphenoxy)pyrazolo[1,5-a]pyrimidine-7-carboxylate

An autoclave vessel was charged with 7-chloro-6-(3-cyclopropylphenoxy)pyrazolo[1,5-a]pyrimidine (0.08 g, 0.28 mmol), triethylamine (0.08 mL, 0.56 mmol), Pd(dppf)Cl₂CH₂Cl₂ (0.057 g, 0.07 mmol) and methanol (20 mL The reactor was flushed three times with carbon monoxide gas and loaded with 10 bar of carbon monoxide. The reaction mixture was heated to 80° C. for 1.5 hr. The progress of the reaction was monitored by LCMS. The reaction mixture was then diluted with water and extracted with EtOAc. The organic layer was washed with brine, dried over sodium sulphate and concentrated under reduced pressure. The resulting crude residue was purified by silica gel chromatography (cyclohexane/EtOAc) to afford methyl 6-(3-cyclopropylphenoxy)pyrazolo[1,5-a]pyrimidine-7-carboxylate as a yellow solid.

LCMS (Method D): retention time 1.10 min, 310 (M+H)

### d) Preparation of lithium 6-(3-cyclopropylphenoxy)pyrazolo[1,5-a]pyrimidine-7-carboxylate

To a solution of methyl 6-(3-cyclopropylphenoxy)pyrazolo[1,5-a]pyrimidine-7-carboxylate (30 mg, 0.096 mmol) in tetrahydrofuran (2 mL) and water (1 mL) was added lithium hydroxide (0.012 g, 0.29 mmol). The reaction was allowed to proceed at rt for 12 hr. The progress of the reaction was monitored by TLC and LCMS. After completion of the reaction, the reaction mixture was concentrated in vacuo to obtain lithium 6-(3-cyclopropylphenoxy)pyrazolo[1,5-a]pyrimidine-7-carboxylate as an off-white solid which was used directly in the next step.

LCMS (Method B): retention time 1.37 min, 296 (M+H)

### e) Preparation of 6-(3-cyclopropylphenoxy)-N-[2-(2,4-dichlorophenyl)-2-fluoro-ethyl]pyrazolo[1,5-a]pyrimidine-7-carboxamide (Compound P-1.3, Table T1)

To lithium 6-(3-cyclopropylphenoxy)pyrazolo[1,5-a]pyrimidine-7-carboxylate (0.02 g, 0.06 mmol) in EtOAc (2 mL), 2-(2,4-dichlorophenyl)-2-fluoro-ethanamine (0.016 g, 0.08 mmol) was added, followed by the addition of 1-propanephosphonic anhydride solution (T3P, 50% in EtOAc, 0.12 g, 0.19 mmol) and triethylamine (0.015 g, 0.14 mmol) at 0° C. This reaction mixture was stirred at rt for 24 hr. The progress of the reaction was monitored by TLC and LCMS. After completion, the reaction mixture was diluted with water and extracted with EtOAc. The organic layer was washed with brine, dried over sodium sulfate and concentrated under reduced pressure. The resulting crude residue was purified by silica gel chromatography (cyclohexane/EtOAc) to afford 6-(3-cyclopropylphenoxy)-N-[2-(2,4-dichlorophenyl)-2-fluoro-ethyl]pyrazolo[1,5-a]pyrimidine-7-carboxamide.

¹H NMR (400 MHz, CDCl₃) δ ppm 9.64 (br s, 1H), 8.43 (s, 1H), 8.19 (d, J=2.50 Hz, 1H), 7.48 (d, J=8.38 Hz, 1H), 7.36-7.43 (m, 1H), 7.18-7.26 (m, 2H), 6.88 (d, J=2.50 Hz, 1H), 6.83 (d, J=7.88 Hz, 1H), 6.72-6.79 (m, 2H), 6.05-5.93 (m, 1H), 4.14-4.24 (m, 1H), 3.80-3.82 (m, 1H), 1.85-1.89 (m, 1H), 0.96-0.98 (m, 2H), 0.67-0.72 (m, 2H).

LCMS (Method C): retention time 1.29 min, 485 (M+H)

### Example P4: This example illustrates the Preparation 7-(3-cyclopropylphenoxy)-N-[2-(2,4-dichlorophenyl)-2-fluoro-ethyl]imidazo[1,2-b]pyridazine-8-carboxamide (Compound P-1.4 of Table T1)

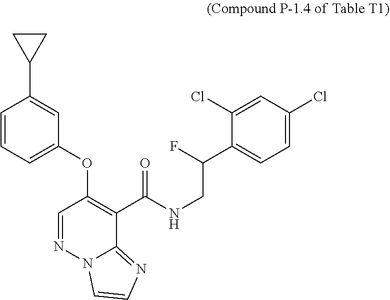 [SMILES: O=C(NCC(F)c1ccc(Cl)cc1Cl)c1c(Oc2cccc(C3CC3)c2)cnn2ccnc12]

### a) Preparation of 2-[(2-bromoimidazol-1-yl)methoxy]ethyl-trimethyl-silane

To a solution of 2-bromo-1H-imidazole (2.0 g, 14 mmol) in tetrahydrofuran (5 mL) at 0° C. was added sodium hydride (60 mass %, 0.65 g, 16 mmol). The reaction mixture was stirred for 30 minutes, then 2-(trimethylsilyl)ethoxymethyl chloride (2.8 mL, 15 mmol) was added at 0° C. The resulting reaction mixture was stirred at rt for 2 hr. The progress of the reaction was monitored by TLC and LCMS. After completion, the reaction mixture was diluted with water and extracted with EtOAc. The organic layer was then washed with brine, dried over sodium sulfate and concentrated under reduced pressure. The resulting crude residue was purified by silica gel chromatography (cyclohexane/EtOAc) to afford 2-[(2-bromoimidazol-1-yl)methoxy]ethyl-trimethyl-silane as a pale yellow liquid.

¹H NMR (400 MHz, CDCl₃) δ ppm 7.11 (d, J=1.63 Hz, 1H), 7.05 (d, J=1.38 Hz, 1H), 5.27 (s, 2H), 3.53 (dd, J=8.69, 7.69 Hz, 2H), 0.87-0.99 (m, 2H), −0.02 (s, 9H)

### b) Preparation of 2-(3-cyclopropylphenoxy)-1-[1-(2-trimethylsilylethoxymethyl)imidazol-2-yl]ethenone

In a two-neck round-bottom flask, 2-[(2-bromoimidazol-1-yl)methoxy]ethyl-trimethyl-silane (2.35 g, 8.50 mmol) was dissolved in tetrahydrofuran (42 mL) and cooled to 0° C. To this, isopropylmagnesium chloride lithium chloride complex solution (1.3 mol/L in THF, 6.5 mL, 8.50 mmol) was added dropwise and stirred for 30 minutes. After that, 2-(3-cyclopropylphenoxy)-N-methoxy-N-methyl-acetamide (1.0 g, 4.25 mmol) was added as a solution in tetrahydrofuran (2 mL). The reaction mixture was stirred at rt for 1 hour. The progress of the reaction was monitored by TLC and LCMS. After completion, the reaction mixture was quenched with a saturated solution of ammonium chloride, and diluted with water. The desired organic material was extracted with EtOAc, dried over sodium sulfate and concentrated under reduced pressure. The resulting crude residue was purified by silica gel chromatography (cyclohexane/EtOAc) to afford 2-(3-cyclopropylphenoxy)-1-[1-(2-trimethylsilylethoxymethyl)imidazol-2-yl]ethenone as a pale yellow liquid.

LCMS (Method B): retention time 1.69 min, 373 (M+H)

2-(3-Cyclopropylphenoxy)-N-methoxy-N-methyl-acetamide used in step b) was prepared as follow:

### i. Preparation of 2-(3-cyclopropylphenoxy)acetic acid

Lithium hydroxide (0.83 g, 19.40 mmol) was added to a solution of methyl 2-(3-cyclopropylphenoxy)acetate (1.48 g, 6.46 mmol) in tetrahydrofuran (9.0 mL) and water (4.5 mL). The reaction was allowed to proceed at rt for 1 hour. The progress of the reaction was monitored by TLC and LCMS. After completion, the reaction mixture was diluted with water and washed with ethyl actetate. The aqueous layer was then acidified with 2N HCl and extracted with EtOAc. The combined organic layers were dried over sodium sulfate and concentrated in vacuo to obtain 2-(3-cyclopropylphenoxy)acetic acid as a beige solid.

¹H NMR (400 MHz, CDCl₃) δ ppm 7.19 (t, J=7.73 Hz, 1H), 6.76 (d, J=7.63 Hz, 1H), 6.64-6.73 (m, 2H), 4.68 (s, 2H), 1.84-1.92 (m, 1H), 0.92-1.02 (m, 2H), 0.64-0.76 (m, 2H).

LCMS (Method B): retention time 1.24 min, 190.8 (M−H)

### ii. Preparation of 2-(3-cyclopropylphenoxy)-N-methoxy-N-methyl-acetamide

To 2-(3-cyclopropylphenoxy)acetic acid (1.34 g, 6.62 mmol) in EtOAc (26.8 mL), methoxy(methyl)ammonium chloride (0.97 g, 9.93 mmol) was added, followed by 1-propanephosphonic anhydride solution (T3P, 50% in EtOAc, 4.64 g, 7.29 mmol) and N,N-diisopropylethylamine (2.59 g, 19.9 mmol). The reaction mixture was stirred at rt for 12 hr. The reaction progress was monitored by TLC and LCMS. After completion, the reaction mixture was diluted with water, extracted with EtOAc and the organic layer was washed with brine, dried over sodium sulfate and concentrated under reduced pressure. The resulting crude residue was purified by silica gel chromatography (10-30% EtOAc in cyclohexane) to afford 2-(3-cyclopropylphenoxy)-N-methoxy-N-methyl-acetamide as a gummy mass.

¹H NMR (400 MHz, CDCl₃) δ ppm 7.16 (t, J=7.74 Hz, 1H), 6.69-6.73 (m, 3H), 4.79 (s, 2H), 3.76 (s, 3H), 3.25 (s, 3H), 1.82-1.92 (m, 1H), 1.67-1.67 (m, 1H), 0.88-1.02 (m, 2H), 0.65-0.74 (m, 2H).

LCMS (Method B): retention time 1.32 min, 236 (M+H)

### c) Preparation of 2-(3-cyclopropylphenoxy)-1-(1H-imidazol-2-yl)ethenone

In a round-bottom flask, 2-(3-cyclopropylphenoxy)-1-[1-(2-trimethylsilylethoxymethyl)imidazol-2-yl]ethanone (0.25 g, 0.47 mmol) was dissolved in methanol (2 mL) and to which hydrochloric acid (1.4 mL, 5.63 mmol) was added. The reaction mixture was stirred at 80° C. for 3 hr. The progress of the reaction was monitored by TLC and LCMS. After the completion, the reaction mixture was concentrated under reduced pressure, diluted with water and extracted with EtOAc. The organic layer was washed with sodium bicarbonate. The combined organic layers were dried under reduced pressure to afford 2-(3-cyclopropylphenoxy)-1-(1H-imidazol-2-yl)ethenone. The resulting crude residue was used as such for the next step.

LCMS (Method C): retention time 1.03 min, 243 (M+H)

### d) Preparation of 2-(3-cyclopropylphenoxy)-3-(dimethylamino)-1-(1H-imidazol-2-yl)prop-2-en-1-one

In a sealed glass reactor, 2-(3-cyclopropylphenoxy)-1-(1H-imidazol-2-yl)ethanone (0.60 g, 2.0 mmol) and 1-tert-butoxy-N,N,N′,N′-tetramethyl-methanediamine (5.0 g, 20.0 mmol) were heated to 90° C. for 2 hr. The progress of the reaction was monitored by LCMS. The reaction mixture was then diluted with water and extracted with EtOAc. The organic layer was washed with brine, dried over sodium sulphate and concentrated under reduced pressure. The resulting crude residue was purified by silica gel chromatography (cyclohexane/EtOAc) to afford 2-(3-cyclopropylphenoxy)-3-(dimethylamino)-1-(1H-imidazol-2-yl)prop-2-en-1-one.

LCMS (Method B): retention time 1.05 min, 298 (M+H)

### e) Preparation of 7-(3-cyclopropylphenoxy)-5H-imidazo[1,2-b]pyridazin-8-one

In a round-bottom flask, 2-(3-cyclopropylphenoxy)-3-(dimethylamino)-1-(1H-imidazol-2-yl)prop-2-en-1-one (0.03 g, 0.10 mmol) was dissolved in N-Methyl-2-pyrrolidone (1 mL). Potassium tert-butoxide (0.012 g, 0.10 mmol) was then added as a suspension in N-Methyl-2-pyrrolidone (0.5 mL). The resulting reaction mixture was stirred at rt for 30 minutes. After that, a solution of amino 4-nitrobenzoate (0.022 g, 0.12 mmol) in N-methyl-2-pyrrolidone (1 mL) was added and reaction mixture was stirred at rt for 18 hr. The progress of the reaction was monitored by LCMS. The reaction mixture was then diluted with water and extracted with EtOAc. The organic layer was washed with brine, dried over sodium sulphate and concentrated under reduced pressure. The resulting crude residue was purified by silica gel chromatography (cyclohexane/EtOAc) to afford a mixture of 1-(1-aminoimidazol-2-yl)-2-(3-cyclopropylphenoxy)-3-(dimethylamino)prop-2-en-1-one and 7-(3-cyclopropylphenoxy)-5H-imidazo[1,2-b]pyridazin-8-one. The mixture was taken as such for the next step.

LCMS (Method B): retention time 0.28 min, 313 (M+H) and retention time 1.9 min, 268 (M+H)

### f) Preparation of 8-chloro-7-(3-cyclopropylphenoxy)imidazo[1,2-b]pyridazine

A mixture of 1-(1-aminoimidazol-2-yl)-2-(3-cyclopropylphenoxy)-3-(dimethylamino)prop-2-en-1-one and 7-(3-cyclopropylphenoxy)-5H-imidazo[1,2-b]pyridazin-8-one (0.4 g), N,N-dimethylaniline (0.7 mL, 5 mmol) and phosphorus(V) oxychloride (8 mL 90 mmol) was stirred at 90° C. for 12 hr. The progress of the reaction was monitored by LCMS. The reaction mixture was then diluted with ice water and extracted with EtOAc. The organic layer was washed with brine, dried over sodium sulphate and concentrated under reduced pressure to obtain a crude residue. The resulting crude residue was purified by silica gel chromatography (cyclohexane/EtOAc) to afford 8-chloro-7-(3-cyclopropylphenoxy)imidazo[1,2-b]pyridazine as a brown gummy mass.

LCMS (Method B): retention time 1.47 min, 286 (M+H)

### g) Preparation of methyl 7-(3-cyclopropylphenoxy)imidazo[1,2-b]pyridazine-8-carboxylate

An autoclave was charged with 8-chloro-7-(3-cyclopropylphenoxy)imidazo[1,2-b]pyridazine (0.13 g, 0.45 mmol), triethylamine (0.13 mL, 0.91 mmol), Pd(dppf)Cl₂CH₂Cl₂ (0.093 g, 0.11 mmol) and methanol (20 mL). The reactor was flushed three times with carbon monoxide gas and loaded with 10 bar of carbon monoxide. The reaction mixture was heated to 80° C. for 1.5 hr. The progress of the reaction was monitored by LCMS. The reaction mixture was then diluted with water and extracted with EtOAc. The organic layer was washed with brine, dried over sodium sulphate and concentrated under reduced pressure. The resulting crude residue was purified by silica gel chromatography (cyclohexane/EtOAc) to afford methyl 7-(3-cyclopropylphenoxy)imidazo[1,2-b]pyridazine-8-carboxylate as a yellow solid.

LCMS (Method B): retention time 2.03 min, 310 (M+H)

### h) Preparation of lithium 7-(3-cyclopropylphenoxy)imidazo[1,2-b]pvridazine-8-carboxylate

To a solution of methyl 7-(3-cyclopropylphenoxy)imidazo[1,2-b]pyridazine-8-carboxylate (0.05 g, 0.16 mmol) in tetrahydrofuran (1 mL) and water (0.05 mL) was added lithium hydroxide monohydrate (20 mg, 0.48 mmol). The reaction was allowed to proceed at rt for 18 hr. The progress of the reaction was monitored by TLC and LCMS. After completion of the reaction, the reaction mixture was concentrated under reduced pressure at 30° C. followed by two successive co-distillations with toluene to afford lithium 7-(3-cyclopropylphenoxy)imidazo[1,2-b]pyridazine-8-carboxylate which was used as such for the next step.

LCMS (Method B): retention time 0.51 min, 296 (M+H)

### i) Preparation of 7-(3-cyclopropylphenoxy)-N-[2-(2,4-dichlorophenyl)-2-fluoro-ethyl]imidazo[1,2-b]pyridazine-8-carboxamide (Compound P-1.4, Table T1)

To lithium 7-(3-cyclopropylphenoxy)imidazo[1,2-b]pyridazine-8-carboxylate (0.0.5 g, 0.16 mmol) in EtOAc (0.83 mL), 2-(2,4-dichlorophenyl)-2-fluoro-ethanamine (0.041 g, 0.19 mmol) was added, followed by addition of 1-propanephosphonic anhydride solution (T3P, 50% in EtOAc, 0.3 mL, 0.50 mmol) and triethylamine (0.05 mL, 0.36 mmol) at 0° C. This reaction mixture was then stirred 2 hr at room temperature. The reaction progress was monitored by TLC and LCMS. After completion, the reaction mixture was diluted with water, extracted with EtOAc and the organic layer was washed with brine, dried over sodium sulfate and concentrated under reduced pressure. The resulting crude residue was purified by silica gel chromatography (cyclohexane/EtOAc) to afford 7-(3-cyclopropylphenoxy)-N-[2-(2,4-dichlorophenyl)-2-fluoro-ethyl]imidazo[1,2-b]pyridazine-8-carboxamide as a white solid.

¹H NMR (400 MHz, CDCl₃) δ ppm 10.15-10.23 (m, 1H), 8.15 (s, 1H), 7.92 (d, J=1.34 Hz, 1H), 7.73 (d, J=1.34 Hz, 1H), 7.52 (d, J=8.44 Hz, 1H), 7.38-7.41 (m, 1H), 7.31-7.35 (m, 1H), 7.27 (m, 1H), 6.89 (d, J=7.83 Hz, 1H), 6.78-6.81 (m, 2H), 6.10-5.95 (m, 1H), 4.10-4.25 (m, 1H), 3.72-3.84 (m, 1H), 1.85-1.92 (m, 1H), 0.84-1.01 (m, 2H), 0.68-0.72 (m, 2H) LCMS (Method B): retention time 1.48 min, 485 (M+H)

### Example P5: This Example Illustrates the Preparation of 6-(3-cyclopropylphenoxy)-N-[2-(2,4-dichlorophenyl)ethyl]-[1,2,4]triazolo[1,5-a]pyrimidine-7-carboxamide (Compound P-1.5 of Table T1)

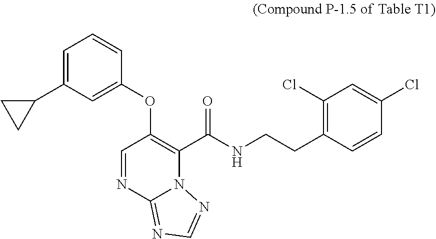 [SMILES: O=C(NCCc1ccc(Cl)cc1Cl)c1c(Oc2cccc(C3CC3)c2)cnc2ncnn12]

6-(3-Cyclopropylphenoxy)-[1,2,4]triazolo[1,5-a]pyrimidine-7-carboxylic acid was prepared as described in example 2, steps a) to d).

### a) Preparation of 6-(3-cyclopropylphenoxy)-[1,2,4]triazolo[1,5-a]pyrimidine-7-carbonyl chloride

To a solution of 6-(3-cyclopropylphenoxy)-[1,2,4]triazolo[1,5-a]pyrimidine-7-carboxylic acid (0.07 g, 0.22 mmol) in dichloromethane (1.4 mL) was added oxalyl chloride (0.03 mL, 0.34 mmol) dropwise under an atmosphere of argon. Then, a drop of dry N,N-dimethyl formamide was added to the reaction mixture. Gas evolution was observed. The reaction mixture was stirred at rt for 1 hour. The reaction mixture was concentrated in vacuo under nitrogen and the brown residue of 6-(3-cyclopropylphenoxy)-[1,2,4]triazolo[1,5-a]pyrimidine-7-carbonyl chloride obtained was used immediately for the next step.

### b) Preparation of 6-(3-cyclopropylphenoxy)-N-[2-(2,4-dichlorophenyl)ethyl]-[1,2,4]triazolo[1,5-a]pyrimidine-7-carboxamide (Compound P-1.5, Table T1)

To a mixture of 2-(2,4-dichlorophenyl)ethanamine (0.04 g, 0.21 mmol) and pyridine (0.04 mL, 0.53 mmol) in acetonitrile (0.85 mL), a solution of 6-(3-cyclopropylphenoxy)-[1,2,4]triazolo[1,5-a]pyrimidine-7-carbonyl chloride (0.07 g, 0.21 mmol) in acetonitrile (0.85 mL) was slowly added. The reaction mixture was stirred overnight at rt for 12 hr. The reaction progress was monitored by TLC and LCMS. After completion, the reaction mixture was diluted with water, extracted with EtOAc and the organic layer was washed with brine, dried over sodium sulfate and concentrated under reduced pressure. The resulting crude residue was purified by reverse-phase chromatography (0-70% acetonitrile in water) to afford 6-(3-cyclopropylphenoxy)-N-[2-(2,4-dichlorophenyl)ethyl]-[1,2,4]triazolo[1,5-a]pyrimidine-7-carboxamide as a white solid.

¹H NMR (400 MHz, CDCl₃) δ ppm 8.55 (s, 1H), 8.35 (s, 1H), 8.29 (br t, J=5.07 Hz, 1H), 7.22 (d, J=2.13 Hz, 1H), 7.04-7.16 (m, 2H), 6.95 (dd, J=8.13, 2.13 Hz, 1H), 6.76 (d, J=7.75 Hz, 1H), 6.58-6.66 (m, 2H), 3.63-3.72 (m, 2H), 2.95 (t, J=6.94 Hz, 2H), 1.70-1.80 (m, 1H), 0.80-0.93 (m, 2H), 0.51-0.62 (m, 2H).

LCMS (Method C): retention time 1.24 min, 468.3 (M+H)

### Example P6: This Example Illustrates the Preparation of 6-(3-cyclopropylphenoxy)-N-[2-(2,4-dichlorophenyl)ethyl]imidazo[1,2-a]pyrimidine-5-carboxamide (Compound P-1.6, Table T1)

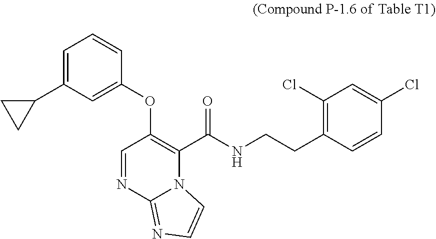 [SMILES: O=C(NCCc1ccc(Cl)cc1Cl)c1c(Oc2cccc(C3CC3)c2)cnc2nccn12]

### a) Preparation of 5-bromo-N-[2-(2,4-dichlorophenyl)ethyl]-2-methylsulfanyl-pyrimidine-4-carboxamide

To 5-bromo-2-methylsulfanyl-pyrimidine-4-carboxylic acid (2.0 g, 8.02 mmol) in EtOAc (20 mL), 2-(2,4-dichlorophenyl)ethanamine (1.67 g, 8.83 mmol) was added, followed by the addition of 1-propanephosphonic anhydride solution (T3P, 50% in EtOAc, 14.3 mL, 24.0 mmol) and triethylamine (2.25 mL, 16.0 mmol) at 0° C. This reaction mixture was stirred at rt for 2 hr. The reaction progress was monitored by TLC and LCMS. After completion, the reaction mixture was diluted with water and extracted with EtOAc. The organic layer was washed with brine, dried over sodium sulfate and concentrated under reduced pressure. The resulting crude residue was purified by silica gel chromatography (cyclohexane/EtOAc) afforded 5-bromo-N-[2-(2,4-dichlorophenyl)ethyl]-2-methylsulfanyl-pyrimidine-4-carboxamide.

LCMS (Method C): retention time 1.18 min, 419.8 (M+H)

### b) Preparation of 5-(3-cyclopropylphenoxy)-N-[2-(2,4-dichlorophenyl)ethyl]-2-methylsulfanyl-pyrimidine-4-carboxamide

In a round-bottom flask 5-bromo-N-[2-(2,4-dichlorophenyl)ethyl]-2-methylsulfanyl-pyrimidine-4-carboxamide (1.3 g, 3.1 mmol) and 3-cyclopropylphenol (0.50 g, 3.7 mmol) were added and dissolved in dimethyl sulfoxide. The reaction mixture was purged with argon for 15 minutes. Tripotassium phosphate (1.3 g, 6.2 mmol), cuprous iodide (0.059 g, 0.31 mmol) and N-benzyl-N′-(2-methyl-1-naphthyl)oxamide (0.098 g, 0.31 mmol) were added and the reaction was flushed with argon. The round-bottom flask was kept on pre-heated block and the reaction mixture was stirred for 15 minutes at 110° C. The mixture was diluted with ice water (30 mL) and extracted with EtOAc (20 mL). The combined organic layer was washed with water (20 mL), followed by brine (30 mL). The combined organic layers were dried over sodium sulphate and concentrated under reduced pressure. The resulting crude residue was purified by silica gel chromatography (cyclohexane/EtOAc) to afford 5-(3-cyclopropylphenoxy)-N-[2-(2,4-dichlorophenyl)ethyl]-2-methylsulfanyl-pyrimidine-4-carboxamide.

LCMS (Method D): retention time 1.29 min, 474 (M+H)

### c) Preparation of 5-(3-cyclopropylphenoxy)-N-[2-(2,4-dichlorophenyl)ethyl]-2-methylsulfonyl-pyrimidine-4-carboxamide

In a round-bottom flask, a mixture of 5-(3-cyclopropylphenoxy)-N-[2-(2,4-dichlorophenyl)ethyl]-2-methylsulfanyl-pyrimidine-4-carboxamide (1.0 g, 2.10 mmol) in dichloromethane (10 mL) was stirred at 0° C. To this solution, 3-chlorobenzenecarboperoxoic acid (1.82 g, 6.32 mmol) was added in portion. The resulting reaction mixture was stirred at rt for 4 hr. The reaction progress was monitored by TLC and LCMS. After completion of the reaction, the reaction mixture was quenched by sodium thiosulphate solution (peroxide amount was monitored by starch paper) and the reaction mixture was diluted with water and extracted with EtOAc. The organic layer was washed with sodium bicarbonate solution, brine solution, dried over sodium sulphate. The resulting crude residue was purified by silica gel chromatography (cyclohexane/EtOAc) to afford 5-(3-cyclopropylphenoxy)-N-[2-(2,4-dichlorophenyl)ethyl]-2-methylsulfonyl-pyrimidine-4-carboxamide.

LCMS (Method C): retention time 1.18 min, 507 (M+H)

### d) Preparation of 2-amino-5-(3-cyclopropylphenoxy)-N-[2-(2,4-ichlorophenyl)ethyl]pyrimidine-4-carboxamide

A premix pressure reactor was charged with 5-(3-cyclopropylphenoxy)-N-[2-(2,4-dichlorophenyl)ethyl]-2-methylsulfonyl-pyrimidine-4-carboxamide (0.3 g, 0.6 mmol) and tetrahydrofuran (0.2 mL). To this, ammonia in 1,4-dioxane (0.5 mol/L, 10 mL) was added and reaction was heated at 90° C. for 5 hr. The reaction progress was monitored by LCMS. After completion of the reaction, the reaction mixture was concentrated under reduced pressure, diluted with water and extracted with EtOAc twice. The combined organic layers were washed with brine, dried over sodium sulphate and concentrated under reduced pressure. The resulting crude residue was purified by silica gel chromatography (cyclohexane/EtOAc) to afford 2-amino-5-(3-cyclopropylphenoxy)-N-[2-(2,4-dichlorophenyl)ethyl]pyrimidine-4-carboxamide.

LCMS (Method C): retention time 1.22 min, 443.3 (M+H)

### e) Preparation of 6-(3-cyclopropylphenoxy)-N-[2-(2,4-dichlorophenyl)ethyl]imidazo[1,2-a]pyrimidine-5-carboxamide (Compound P-1.6. Table T1)

A microwave vessel charged with 2-amino-5-(3-cyclopropylphenoxy)-N-[2-(2,4-dichlorophenyl)ethyl]pyrimidine-4-carboxamide (0.16 g, 0.36 mmol), ethanol (2 mL) and 2-chloroacetaldehyde (0.085 g, 1.1 mmol). The reaction was irradiated at 98° C. for 12 hr. The reaction progress was monitored by LCMS. After completion, the reaction mixture was concentrated under reduced pressure, diluted with water and extracted with EtOAc twice. The combined organic layers were washed with brine, dried over sodium sulphate and concentrated under reduced pressure. The crude residue was purified by silica gel chromatography (cyclohexane/EtOAc) to afford 6-(3-cyclopropylphenoxy)-N-[2-(2,4-dichlorophenyl)ethyl]imidazo[1,2-a]pyrimidine-5-carboxamide (0.045 g, 27%) and 6-(3-cyclopropylphenoxy)-N-[2-(2,4-dichlorophenyl)ethyl]imidazo[1,2-a]pyrimidine-7-carboxamide.

¹H NMR (400 MHz, CDCl₃) δ ppm 8.83 (s, 1H), 8.30 (s, 1H), 7.94 (s, 1H), 7.64 (br s, 1H), 7.26-7.28 (m, 2H), 7.02 (d, J=8.13 Hz, 1H), 6.91-6.97 (m, 2H), 6.62-6.75 (m, 2H), 3.78-3.86 (m, 2H), 2.96-3.12 (m, 2H), 1.86-1.97 (m, 1H), 1.03-1.05 (m, 2H), 0.70-0.72 (m, 2H).

LCMS (Method D): retention time 1.15 min, 467.0 (M+H)

### Example P7: This Example Illustrates the Preparation of 6-(3-cyclopropylphenoxy)-N-[2-(2,4-dichlorophenyl)-2-fluoro-ethyl]-2-methyl-imidazo[1,2-a]pyrimidine-5-carboxamide (Compound P-1.7, Table T1)

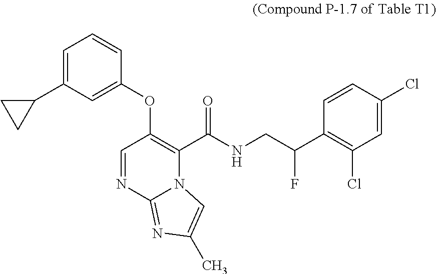 [SMILES: Cc1cn2c(C(=O)NCC(F)c3ccc(Cl)cc3Cl)c(Oc3cccc(C4CC4)c3)cnc2n1]

Ethyl 2-(3-cyclopropylphenoxy)-3-(dimethylamino)prop-2-enoate was prepared as described in example 1, steps a) and b).

### a) Preparation of 6-(3-cyclopropylphenoxy)-2-methyl-8H-imidazo[1,2-a]pyrimidin-5-one

In a single-neck round-bottom flask, a mixture of ethyl 2-(3-cyclopropylphenoxy)-3-(dimethylamino)prop-2-enoate (2.00 g, 6.90 mmol), 5-methyl-1H-imidazol-2-amine; hydrochloride (0.92 g, 6.90 mmol) and sodium acetate (0.57 g, 6.90 mmol) in acetic acid (20 mL) was stirred at 100° C. for 18 hr. Progress of the reaction was monitored by LCMS. After completion of the reaction, the reaction mixture was cooled to room temperature, diluted with ice-cold water and extracted with EtOAc. The organic layer was dried over sodium sulphate and concentrated in vacuum to obtain a crude residue, which was purified by silica gel chromatography (cyclohexane/EtOAc) to afford 6-(3-cyclopropylphenoxy)-2-methyl-8H-imidazo[1,2-a]pyrimidin-5-one as a white solid.

LCMS (Method B): retention time 1.31 min, 282 (M+H)

### b) Preparation of 5-chloro-6-(3-cyclopropylphenoxy)-2-methyl-imidazo[1,2-a]pyrimidine

In a single-neck round-bottom flask, to a mixture of 6-(3-cyclopropylphenoxy)-2-methyl-8H-imidazo[1,2-a]pyrimidin-5-one (1.00 g, 3.55 mmol) and phosphorus(V) oxychloride (25 mL), pyridine (1 mL) was added at 0° C. under stirring. The mixture was allowed to come to rt and then stirred at 70° C. for 5 hr. Progress of the reaction was monitored by LCMS. After completion of the reaction, the reaction mixture was concentrated in vacuum and quenched by adding it to crushed ice. The organic layer was washed with 150 mL saturated sodium bicarbonate solution, 150 mL water and finally with brine solution, dried over sodium sulphate and concentrated under reduced pressure to obtain a crude residue. The resulting crude residue was purified by silica gel chromatography (0-20% EtOAc in cyclohexane) to afford 5-chloro-6-(3-cyclopropylphenoxy)-2-methyl-imidazo[1,2-a]pyrimidine as a pale yellow gummy mass.

LCMS (Method C): retention time 1.12 min, 300 (M+H)

### c) Preparation of methyl 6-(3-cyclopropylphenoxy)-2-methyl-imidazo[1,2-a]pyrimidine-5-carboxylate

An autoclave vessel was charged with 5-chloro-6-(3-cyclopropylphenoxy)-2-methyl-imidazo[1,2-a]pyrimidine (0.48 g, 1.60 mmol), triethylamine (0.45 mL, 3.20 mmol), Pd(dppf)Cl₂CH₂Cl₂ (0.33 g, 0.40 mmol) in methanol (32 mL). The vessel was flushed three times with carbon monoxide gas and loaded with 10 bar of carbon monoxide. The reaction mixture was heated to 80° C. for 3 hr. The progress of the reaction was monitored by LCMS. After completion of the reaction, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (0-30% EtOAc in cyclohexane) to afford methyl 6-(3-cyclopropylphenoxy)-2-methyl-imidazo[1,2-a]pyrimidine-5-carboxylate as a yellow gummy mass.

LCMS (Method D): retention time 1.09 min, 324 (M+H)

### d) Preparation of 6-(3-cyclopropylphenoxy)-N-[2-(2,4-dichlorophenyl)-2-fluoro-ethyl]-2-methyl-imidazo[1,2-a]pyrimidine-5-carboxamide (Compound P-1.7, Table T1)

A 10 mL microwave vial was charged with methyl 6-(3-cyclopropylphenoxy)-2-methyl-imidazo[1,2-a]pyrimidine-5-carboxylate (0.10 g, 0.30 mmol) and 2-(2,4-dichlorophenyl)-2-fluoro-ethanamine (0.06 g, 0.30 mmol) and the vial was sealed and irradiated with microwave under stirring at 100° C. for 2 hr. Progress of the reaction was monitored by TLC and LCMS. After completion, the reaction mixture was diluted with water and extracted with EtOAc. The resulting organic layer was washed with brine, dried over sodium sulphate, and concentrated under reduced pressure. The resulting crude residue was purified by reverse phase combi flash by using 0-70% acetonitrile in water as an eluent to afford 6-(3-cyclopropylphenoxy)-N-[2-(2,4-dichlorophenyl)-2-fluoro-ethyl]-2-methyl-imidazo[1,2-a]pyrimidine-5-carboxamide as a yellow solid.

¹H NMR (400 MHz, CHLOROFORM-d) δ ppm 8.52 (s, 1H), 8.28 (s, 1H), 7.96 (br s, 1H), 7.20-7.37 (m, 3H), 7.09 (dd, J=8.38, 2.02 Hz, 1H), 6.93-6.97 (m, 1H), 6.88 (m, 1H), 6.72-6.81 (m, 1H), 5.81-6.04 (m, 1H), 4.04-4.18 (m, 1H), 3.84-3.97 (m, 1H), 2.55 (s, 3H), 1.76-2.04 (m, 1H), 1.00-1.06 (m, 2H), 0.69-0.74 (m, 2H).

LCMS (Method C): retention time 1.22 min, 499 (M+H)

### Example P8: This Example Illustrates the Preparation of 2-cyano-6-(3-cyclopropylphenoxy)-N-[2-(2,4-dichlorophenyl)-2-fluoro-ethyl]pyrazolo[1,5-a]pyrimidine-7-carboxamide (Compound P-1.8. Table T1)

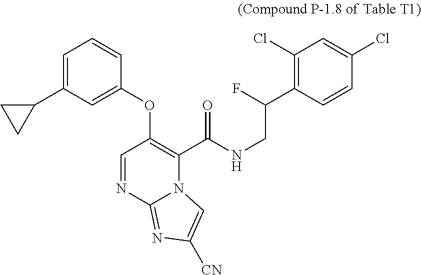 [SMILES: N#Cc1cn2c(C(=O)NCC(F)c3ccc(Cl)cc3Cl)c(Oc3cccc(C4CC4)c3)cnc2n1]

Ethyl 2-(3-cyclopropylphenoxy)-3-(dimethylamino)prop-2-enoate was prepared as described in example 1.

### a) Preparation of 2-bromo-6-(3-cyclopropylphenoxy)-4H-pyrazolo[1,5-a]pyrimidin-7-one

In a single-neck round bottom flask, a mixture of ethyl 2-(3-cyclopropylphenoxy)-3-(dimethylamino)prop-2-enoate (0.5 g, 3.45 mmol), 3-bromo-1H-pyrazol-5-amine (0.57 g, 3.45 mmol) and sodium acetate (0.28 g, 3.45 mmol) in acetic acid (10 mL) was stirred at 100° C. for 1 h. The reaction mixture was cooled down to 60° C., magnesium chloride (0.65 g, 6.90 mmol) was added, and heated to 120° C. for approximately 4 hr.

Progress of the reaction was monitored by LCMS analysis. The reaction mixture was cooled to rt, and water (30 mL) was added. The resulting precipitate was filtered to afford 2-bromo-6-(3-cyclopropylphenoxy)-4H-pyrazolo[1,5-a]pyrimidin-7-one.

LCMS (Method A): retention time 0.87 min, 348 (M+3H)

### b) Preparation of 6-(3-cyclopropylphenoxy)-7-oxo-4H-pyrazolo[1,5-a]pyrimidine-2-carbonitrile

A 30 mL-vial was charged with zinc cyanide (0.43 g, 3.65 mmol), Pd2(dba)₃ (1.57 g, 1.66 mmol), Pd(dppf)Cl₂ (0.25 g, 0.332 mmol) and Zn powder (54.4 mg, 0.83 mmol). A solution of 2-bromo-6-(3-cyclopropylphenoxy)-4H-pyrazolo[1,5-a]pyrimidin-7-one (1.15 g, 3.32 mmol) in dry DMA (17 mL) was added. The mixture was purged with argon. The vial was closed and the reaction mixture was stirred under heating at 120° C. for 2 hr. Progress of the reaction was monitored by LCMS. The reaction mixture was then quenched with water, filtered over a celite pad, and diluted with EtOAc. The organic layer was washed with water (5×40 mL). A chromatography on C18 120 g (water/20-70% acetonitrile) was carried out to afford 6-(3-cyclopropylphenoxy)-7-oxo-4H-pyrazolo[1,5-a]pyrimidine-2-carbonitrile.

LCMS (Method A): retention time 0.85 min, 293 (M+H)

### c) Preparation of 7-chloro-6-(3-cyclopropylphenoxy)pyrazolo[1,5-a]pyrimidine-2-carbonitrile

A 100 mL round-bottom flask was taken 6-(3-cyclopropylphenoxy)-7-oxo-4H-pyrazolo[1,5-a]pyrimidine-2-carbonitrile (0.32 g, 1.1 mmol) and POCl₃ (16 mL, 170 mmol) was added to it under stirring. Pyridine (0.18 mL, 2.2 mmol) was added slowly at rt and the mixture was stirred to 90° C. for 20 hr. Progress of the reaction was monitored by LCMS, showing full conversion of product. The reaction mixture was concentrated to dryness under vacuum, then diluted in EtOAc, washed three times with water, once with brine, then dried over magnesium sulfate, filtered, and concentrated on a rotavapor. The crude product 7-chloro-6-(3-cyclopropylphenoxy)pyrazolo[1,5-a]pyrimidine-2-carbonitrile was taken forward in the next step without further purification.

LCMS (Method A): retention time 1.13 min, 311 (M+H)

### d) Preparation of methyl 2-cyano-6-(3-cyclopropylphenoxy)pyrazolo[1,5-a]pyrimidine-7-carboxylate

An autoclave vessel was charged with 7-chloro-6-(3-cyclopropylphenoxy)pyrazolo[1,5-a]pyrimidine-2-carbonitrile (0.35 g, 1.10 mmol), triethylamine (0.32 mL, 4.61 mmol), Pd(dppf)Cl₂CH₂Cl₂ (0.19 g, 0.23 mmol) in methanol (23 mL). The reactor was flushed with nitrogen gas, then twice with carbon monoxide gas, then loaded with 10 bar of carbon monoxide. The reaction mixture was heated to 80° C. for 2 hr. Progress of the reaction was monitored by LCMS. After completion of the reaction, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford methyl 2-cyano-6-(3-cyclopropylphenoxy)pyrazolo[1,5-a]pyrimidine-7-carboxylate.

LCMS (Method A): retention time 1.08 min, 335 (M+H)

### e) Preparation of lithium; 2-cyano-6-(3-cyclopropylphenoxy)pyrazolo[1,5-a]pyrimidine-7-carboxylate

To a solution of methyl 2-cyano-6-(3-cyclopropylphenoxy)pyrazolo[1,5-a]pyrimidine-7-carboxylate (0.20 g, 0.60 mmol) in tetrahydrofuran (18 mL) and water (6 mL) was added lithium; hydroxide; hydrate (51 mg, 1.2 mmol). The reaction mixture was stirred at rt for 1 hour. Progress of the reaction was monitored by LCMS and TLC. After completion of reaction, the reaction mixture was concentrated under reduced pressure at 30° C. followed twice with co-distillation with toluene. The crude lithium 2-cyano-6-(3-cyclopropylphenoxy)pyrazolo[1,5-a]pyrimidine-7-carboxylate salt was taken as such for the next step.

LCMS (Method A): retention time 0.75 min, 321 (M+H)

### f) Preparation of 2-cyano-6-(3-cyclopropylphenoxy)-N-[2-(2,4-dichlorophenyl)-2-fluoro-ethyl]pyrazolo[1,5-a]pyrimidine-7-carboxamide (Compound P-1.8, Table T1)

A solution of lithium; 2-cyano-6-(3-cyclopropylphenoxy)pyrazolo[1,5-a]pyrimidine-7-carboxylate salt (0.20 g, 0.613 mmol), (1-Cyano-2-ethoxy-2-oxoethylidenaminooxy)dimethylamino-morpholino-carbenium hexafluorophosphate (0.32 g, 0.74 mmol) were stirred in EtOAc (6 mL) at room temperature. Diisopropylethylamine (0.32 mL, 1.84 mmol) was added, followed by addition of 2-(2,4-dichlorophenyl)-2-fluoroethan-1-amine (0.14 g, 0.67 mmol). Progress of the reaction was monitored by LCMS and TLC. After completion of reaction, the reaction mixture was diluted with water. It was then extracted twice with EtOAc. The combined organic layers were washed once with brine, dried over anhydrous sodium sulfate, filtered, and concentrated under reduced pressure at 60° C. The resulting crude residue was purified by chromatography on C18 40 g (30-80% ACN in water+0.1% formic acid) to give 2-cyano-6-(3-cyclopropylphenoxy)-N-[2-(2,4-dichlorophenyl)-2-fluoro-ethyl]pyrazolo[1,5-a]pyrimidine-7-carboxamide.

¹H NMR (400 MHz, CDCl₃) δ ppm 8.-1-8.53 (m, 1H), 8.-7-8.27 (m, 1H), 7.-1-7.42 (m, 2H), 7.32 (dd, J=49.59, 8.54 Hz, 1H), 7.-3-7.22 (m, 3H), 6.-7-6.95 (m, 2H), 5.90 (ddd, J=46.32, 6.72, 3.27 Hz, 1H), 4.-1-4.20 (m, 1H), 3.-8-3.85 (m, 1H), 3.-6-3.50 (m, 1H), 2.-3-2.96 (m, 1H), 2.-9-2.82 (m, 1H), 1.-2-2.04 (m, 1H), 1.81 (tt, J=8.36, 5.09 Hz, 1H), 1.-4-1.28 (m, 2H), 0.-4-1.00 (m, 2H) 0.-3-0.72 (m, 2H)

LCMS (Method A): retention time 1.19 min, 510 (M+H)

### Example P9: This Example Illustrates the Preparation of 3-chloro-6-(3-cyclopropylphenoxy)-N-[2-(2,4-dichlorophenyl)-2-fluoro-ethyl]pyrazolo[1,5-a]pyrimidine-7-carboxamide (Compound P-1.9, Table T1)

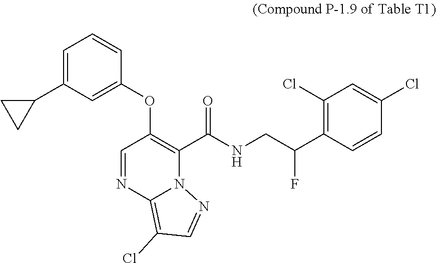 [SMILES: O=C(NCC(F)c1ccc(Cl)cc1Cl)c1c(Oc2cccc(C3CC3)c2)cnc2c(Cl)cnn12]

Ethyl 2-(3-cyclopropylphenoxy)-3-(dimethylamino)prop-2-enoate was prepared as described in example 1.

### a) Preparation of 3-chloro-6-(3-cyclopropylphenoxy)-4H-pyrazolo[1,5-a]pyrimidin-7-one

In a single-neck round-bottom flask, a mixture of ethyl 2-(3-cyclopropylphenoxy)-3-(dimethylamino)prop-2-enoate (1.50 g, 5.20 mmol), 4-chloro-1H-pyrazol-3-amine (0.61 g, 5.20 mmol) and sodium acetate (0.43 g, 5.20 mmol) in acetic acid (15 mL) was stirred at 100° C. for 12 hr. The progress of the reaction was monitored by LCMS. After completion, the reaction mixture was diluted with ice cold water and stirred for 15 minutes. The solid obtained was filtered over Buchner funnel and dried under vacuum to afford 3-chloro-6-(3-cyclopropylphenoxy)-4H-pyrazolo[1,5-a]pyrimidin-7-one as a brown solid.

LCMS (Method B): retention time 1.34 min, 302 (M+H)

### b) Preparation of 3,7-dichloro-6-(3-cyclopropylphenoxy)pyrazolo[1,5-a]pyrimidine

In a single-neck round-bottom flask, a mixture of 3-chloro-6-(3-cyclopropylphenoxy)-4H-pyrazolo[1,5-a]pyrimidin-7-one (0.80 g, 2.65 mmol), and phosphorus(V) oxychloride (18.8 mL, 201 mmol) was added.

The reaction mixture was cooled to 0° C. and pyridine (0.86 mL, 10.6 mmol) was added dropwise via syringe at 0° C. and the resulting reaction mixture was stirred at rt and then at 85° C. for 16 hr. The progress of the reaction was monitored by LCMS. After completion, the reaction mixture was cooled to room temperature, concentrated in vacuo to minimum volume and quenched by adding it to crushed ice and the organic desired material was extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford 3,7-dichloro-6-(3-cyclopropylphenoxy)pyrazolo[1,5-a]pyrimidine as a yellow gummy mass.

LCMS (Method B): retention time 1.73 min, 320 (M+H)

### c) Preparation of methyl 3-chloro-6-(3-cyclopropylphenoxy)pyrazolo[1,5-a]pyrimidine-7-carboxylate

An autoclave vessel was charged with 3,7-dichloro-6-(3-cyclopropylphenoxy)pyrazolo[1,5-a]pyrimidine (0.50 g, 1.56 mmol), triethylamine (0.44 mL, 3.12 mmol), Pd(dppf)Cl₂CH₂Cl₂ (0.32 g, 0.39 mmol) in methanol (46.8 mL). The reactor was flushed three times with carbon monoxide gas and loaded with 10 bar of carbon monoxide. The reaction mixture was heated to 80° C. for 3 hr. Progress of the reaction was monitored by LCMS. After completion, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford methyl 3-chloro-6-(3-cyclopropylphenoxy)pyrazolo[1,5-a]pyrimidine-7-carboxylate as a yellow gummy mass.

LCMS (Method B): retention time 1.67 min, 344 (M+H)

### d) Preparation of 3-chloro-6-(3-cyclopropylphenoxy)-N-[2-(2,4-dichlorophenyl)-2-fluoro-ethyl]pyrazolo[1,5-a]pyrimidine-7-carboxamide (Compound P-1.9, Table T1)

A 10 mL microwave vial was charged with methyl 3-chloro-6-(3-cyclopropylphenoxy)pyrazolo[1,5-a]pyrimidine-7-carboxylate (110 mg, 0.32 mmol) and 2-(2,4-dichlorophenyl)-2-fluoro-ethanamine (133 mg, 0.64 mmol). Then the vial was sealed and irradiated with microwave under stirring at 90° C. for 2 hr. Progress of the reaction was monitored by TLC and LCMS. After completion, the reaction mixture was diluted with water and extracted with EtOAc. The resulting organic layer was washed with brine, dried over sodium sulphate, and concentrated under reduced pressure. The resulting crude residue was purified by silica gel chromatography (cyclohexane/EtOAc as an eluent) to afford 3-chloro-6-(3-cyclopropylphenoxy)-N-[2-(2,4-dichlorophenyl)-2-fluoro-ethyl]pyrazolo[1,5-a]pyrimidine-7-carboxamide as an off-white solid.

¹H NMR (400 MHz, CDCl₃) δ ppm 9.02 (br s, 1H), 8.47 (s, 1H), 8.16 (s, 1H), 7.45 (d, J=8.44 Hz, 1H), 7.40 (s, 1H), 7.20-7.27 (m, 2H), 6.86 (d, J=7.83 Hz, 1H), 6.71-6.77 (m, 2H), 5.81-6.11 (m, 1H), 4.12-4.26 (m, 1H), 3.74-3.87 (m, 1H), 1.83-1.91 (m, 1H), 0.95-1.01 (m, 2H), 0.66-0.73 (m, 2H)

¹⁹F NMR (377 MHz, CDCl₃) δ ppm 188.27 (s, 1 F)

LCMS (Method C): retention time 1.25 min, 517 (M−H)

An analytical sample rac-3-chloro-6-(3-cyclopropylphenoxy)-N-[2-(2,4-dichlorophenyl)-2-fluoro-ethyl]pyrazolo[1,5-a]pyrimidine-7-carboxamide was analysed by Chiral phase column chromatography.

Method: SFC:Waters Acquity UPC²/QDa PDA Detector Waters Acquity UPC² Column: Daicel SFC CHIRALPAK® AY, 3 m, 0.3 cm×10 cm, 40° C. Mobile phase: A: CO₂ B: IPA isocratic: 25% B ABPR: 1800 psi Flow rate: 2.0 ml/min Detection: 240 nm Sample concentration: 1 mg/mL in MeOH/ACN Injection: 2 μl.

This gave as first eluting isomer 3-chloro-6-(3-cyclopropylphenoxy)-N-[(2R)-2-(2,4-dichlorophenyl)-2-fluoro-ethyl]pyrazolo[1,5-a]pyrimidine-7-carboxamide (Retention time 3.39 min) and as second eluting isomer 3-chloro-6-(3-cyclopropylphenoxy)-N-[(2S)-2-(2,4-dichlorophenyl)-2-fluoro-ethyl]pyrazolo[1,5-a]pyrimidine-7-carboxamide (ret time 5.59 min). Preparative samples were obtained over a preparative chiral column:

Preparative method: Sepiatec Prep SFC 100; Column: Daicel CHIRALPAK® AY, 5□m, 2.0 cm×25 cm Mobile phase: A: CO₂ B: EtOH isocratic: 25% B; Backpressure: 150 bar; GLS: —; Flow rate: 60 ml/min, Detection: UV 240 nm; Sample concentration: 1 g in 33 ml IPA/ACN/DCM (10/10/13); Injection: 950 μl

The first eluting peak (ret. Time ca. 2.5 min) corresponds to 3-chloro-6-(3-cyclopropylphenoxy)-N-[(2R)-2-(2,4-dichlorophenyl)-2-fluoro-ethyl]pyrazolo[1,5-a]pyrimidine-7-carboxamide, whilst the second eluting peak (ret time 3.9 min) corresponds to 3-chloro-6-(3-cyclopropylphenoxy)-N-[(2S)-2-(2,4-dichlorophenyl)-2-fluoro-ethyl]pyrazolo[1,5-a]pyrimidine-7-carboxamide. The analytical ee of bothe sample as measured on the analytical column described above was >98%.

### Example P10: This Example Illustrates the Preparation of 6-(3-cyclopropylphenoxy)-N-[2-(2,4-dichlorophenyl)-2-fluoro-ethyl]-2-fluoro-pyrazolo[1,5-a]pyrimidine-7-carboxamide (Compound P-1.10, Table T1)

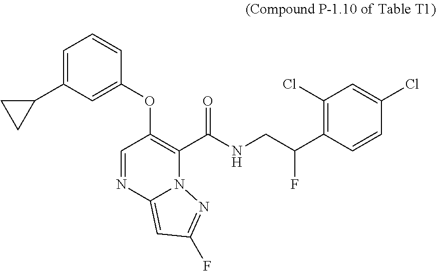 [SMILES: O=C(NCC(F)c1ccc(Cl)cc1Cl)c1c(Oc2cccc(C3CC3)c2)cnc2cc(F)nn12]

Ethyl 2-(3-cyclopropylphenoxy)-3-(dimethylamino)prop-2-enoate was prepared as described in example 1.

### a) Preparation of 6-(3-cyclopropylphenoxy)-2-fluoro-4H-pyrazolo[1,5-a]pyrimidin-7-one

In a single-neck round-bottom flask, a mixture of ethyl 2-(3-cyclopropylphenoxy)-3-(dimethylamino)prop-2-enoate (0.5 g, 1.73 mmol) (prepared in example 1), 5-fluoro-1H-pyrazol-3-amine (0.17 g, 1.73 mmol) and sodium acetate (0.14 g, 1.73 mmol) in acetic acid (5 mL) was stirred at 100° C. for 16 hr. Progress of the reaction was monitored by LCMS. After completion, the reaction mixture was cooled to rt, and diluted with ice-cold water. The resulting solid compound was then precipitated, filtered on a Buchner funnel, washed with cold water, and dried to afford 6-(3-cyclopropylphenoxy)-2-fluoro-4H-pyrazolo[1,5-a]pyrimidin-7-one as an off-white solid.

LCMS (Method B): retention time 1.32 min, 284 (M−H)

### b) Preparation of 7-chloro-6-(3-cyclopropylphenoxy)-2-fluoro-pyrazolo[1,5-a]pyrimidine

In a single-neck round-bottom flask, to a mixture of 6-(3-cyclopropylphenoxy)-2-fluoro-4H-pyrazolo[1,5-a]pyrimidin-7-one (1.5 g, 5.25 mmol) and phosphorus(V) oxychloride (37.3 mL 399 mmol), was added pyridine (1.71 mL, 21.0 mmol) at 0° C. The reaction mixture was stirred at 85° C. for 12 hr. Progress of the reaction was monitored by LCMS. After completion, the reaction mixture was concentrated to minimum volume and then diluted with ice water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford 7-chloro-6-(3-cyclopropylphenoxy)-2-fluoro-pyrazolo[1,5-a]pyrimidine as a pale yellow solid.

LCMS (Method B): retention time 1.70 min, 304 (M+H)

### c) Preparation of methyl 6-(3-cyclopropylphenoxy)-2-fluoro-pyrazolo[1,5-a]pyrimidine-7-carboxylate

An autoclave vessel was charged with 7-chloro-6-(3-cyclopropylphenoxy)-2-fluoro-pyrazolo[1,5-a]pyrimidine (0.7 g, 2.30 mmol), triethylamine (0.65 mL, 4.61 mmol), Pd(dppf)Cl₂CH₂Cl₂ (0.47 g, 0.57 mmol) in methanol (69 mL). The reactor was flushed three times with carbon monoxide gas and loaded with 10 bar of carbon monoxide. The reaction mixture was heated to 80° C. for 3 hr. Progress of the reaction was monitored by LCMS. After completion, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford methyl 6-(3-cyclopropylphenoxy)-2-fluoro-pyrazolo[1,5-a]pyrimidine-7-carboxylate as a yellow gummy mass.

LCMS (Method B): retention time 1.65 min, 328 (M+H)

### d) Preparation of 6-(3-cyclopropylphenoxy)-N-[2-(2,4-dichlorophenyl)-2-fluoro-ethyl]-2-fluoro-pyrazolo[1,5-a]pyrimidine-7-carboxamide (Compound P-1.10, Table T1)

A 10 mL microwave vial was charged with methyl 3-chloro-6-(3-cyclopropylphenoxy)pyrazolo[1,5-a]pyrimidine-7-carboxylate (100 mg, 0.30 mmol) and 2-(2,4-dichlorophenyl)-2-fluoro-ethanamine (127 mg, 0.61 mmol). Then the vial was sealed and irradiated with microwave under stirring at 90° C. for 2 hr. Progress of the reaction was monitored by TLC and LCMS. After completion, the reaction mixture was diluted with water and extracted with EtOAc. The resulting organic layer was washed with brine, dried over sodium sulphate, and concentrated under reduced pressure. The resulting crude residue was purified by silica gel chromatography (cyclohexane/EtOAc as an eluent) to afford 6-(3-cyclopropylphenoxy)-N-[2-(2,4-dichlorophenyl)-2-fluoro-ethyl]-2-fluoro-pyrazolo[1,5-a]pyrimidine-7-carboxamide as a white solid.

¹H NMR (400 MHz, CDCl₃) δ ppm 8.39-8.52 (m, 2H), 7.49 (d, J=8.38 Hz, 1H), 7.37-7.44 (m, 1H), 7.21-7.28 (m, 2H), 6.86 (d, J=7.75 Hz, 1H), 6.71-6.78 (m, 2H), 6.40 (d, J=5.13 Hz, 1H), 5.89-6.04 (m, 1H), 4.13-4.26 (m, 1H), 3.76-3.89 (m, 1H), 1.89 (tt, J=8.41, 5.03 Hz, 1H), 0.89-1.05 (m, 2H), 0.67-0.74 (m, 2H) ¹⁹F NMR (377 MHz, CDCl₃) δ ppm 117.63 (s, 1 F), −188.54 (s, 1 F) LCMS (Method C): retention time 1.29 min, 503 (M+H)

### Example P11: This Example Illustrates the Preparation of 6-(3-cyclopropylphenoxy)-N-[2-(2,4-dichlorophenyl)-2-fluoro-ethyl]-3-methyl-pyrazolo[1,5-a]pyrimidine-7-carboxamide (Compound P-1.11, Table T1)

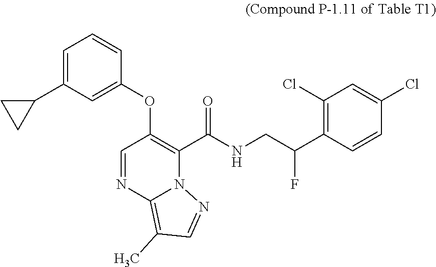 [SMILES: Cc1cnn2c(C(=O)NCC(F)c3ccc(Cl)cc3Cl)c(Oc3cccc(C4CC4)c3)cnc12]

Ethyl 2-(3-cyclopropylphenoxy)-3-(dimethylamino)prop-2-enoate was prepared as described in example 1.

### a) Preparation of 6-(3-cyclopropylphenoxy)-3-methyl-4H-pyrazolo[1,5-a]pyrimidin-7-one

In a single-neck round-bottom flask, the mixture of ethyl 2-(3-cyclopropylphenoxy)-3-(dimethylamino)prop-2-enoate (0.30 g, 1.09 mmol), 4-methyl-1H-pyrazol-3-amine (0.73 g, 7.59 mmol) and sodium acetate (90.3 mg, 1.09 mmol) in acetic acid (3 mL) was stirred at 100° C. for 16 hr. The progress of the reaction was monitored by LCMS. After completion, the reaction mixture was cooled to rt, and diluted with ice-cold water. The solid obtained was filtered and dried over vacuum to afford 6-(3-cyclopropylphenoxy)-3-methyl-4H-pyrazolo[1,5-a]pyrimidin-7-one as a brown solid.

LCMS (Method B): retention time 1.28 min, 282 (M+H)⁺

### b) Preparation of 7-chloro-6-(3-cyclopropylphenoxy)-3-methyl-pyrazolo[1,5-a]pyrimidine

In a single-neck round-bottom flask, a mixture of 6-(3-cyclopropylphenoxy)-2-methyl-4H-pyrazolo[1,5-a]pyrimidin-7-one (170 mg, 0.60 mmol) and phosphorus(V) oxychloride (4.3 mL, 45.9 mmol) was cooled to 0° C., and pyridine (19.7 μL, 2.41 mmol) was added dropwise via syringe at 0° C. The resulting reaction mixture was stirred at 80° C. for 2 hr. Progress of the reaction was monitored by LCMS. After completion, the reaction mixture was concentrated under reduced pressure, the reaction mixture was diluted with ice water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford 7-chloro-6-(3-cyclopropylphenoxy)-3-methyl-pyrazolo[1,5-a]pyrimidine as a pale yellow gummy mass.

LCMS (Method B): retention time 1.79 min, 300 (M+H)

### c) Preparation of methyl 6-(3-cyclopropylphenoxy)-3-methyl-pyrazolo[1,5-a]pyrimidine-7-carboxylate

An autoclave vessel was charged with 7-chloro-6-(3-cyclopropylphenoxy)-3-methyl-pyrazolo[1,5-a]pyrimidine (0.14 g, 0.46 mmol), triethylamine (0.13 mL, 0.93 mmol), Pd(dppf)Cl₂CH₂Cl₂ (93.3 mg, 0.11 mmol) in methanol (20 mL). The reactor was flushed three times with carbon monoxide gas and loaded with 10 bar of carbon monoxide. The reaction mixture was heated to 80° C. for 5 hr. Progress of the reaction was monitored by LCMS. After completion, the reaction mixture was cooled, filtered through Celite and concentrated in vacuo to obtain a reddish-brown crude residue. The resulting crude residue was purified by silica gel chromatography (cyclohexane/EtOAc) to afford methyl 6-(3-cyclopropylphenoxy)-3-methyl-pyrazolo[1,5-a]pyrimidine-7-carboxylate as a yellow gummy mass.

LCMS (Method B): retention time 1.63 min, 324 (M+H)

### d) Preparation of 6-(3-cyclopropylphenoxy)-N-[2-(2,4-dichlorophenyl)-2-fluoro-ethyl]-3-methyl-pyrazolo[1,5-a]pyrimidine-7-carboxamide (Compound P-1.11, Table T1)

A 10 mL microwave vial was charged with methyl 6-(3-cyclopropylphenoxy)-3-methyl-pyrazolo[1,5-a]pyrimidine-7-carboxylate (70.0 mg, 0.22 mmol) and 2-(2,4-dichlorophenyl)-2-fluoro-ethanamine (45.0 mg, 0.22 mmol). The vial was then sealed and irradiated with microwave under stirring at 100° C. for 2 hr. Progress of the reaction was monitored by TLC and LCMS. After completion, the reaction mixture was diluted with water, and extracted with EtOAc. The resulting organic layer was washed with brine, dried over sodium sulphate, and concentrated under reduced pressure. The resulting crude residue was purified by silica gel chromatography (cyclohexane/EtOAc as an eluent) to afford 6-(3-cyclopropylphenoxy)-N-[2-(2,4-dichlorophenyl)-2-fluoro-ethyl]-3-methyl-pyrazolo[1,5-a]pyrimidine-7-carboxamide.

¹H NMR (400 MHz, CDCl₃) δ ppm 9.77 (br s, 1H), 8.28 (s, 1H), 7.94 (s, 1H), 7.39 (d, J=8.31 Hz, 1H), 7.28-7.35 (m, 1H), 7.17-7.22 (m, 1H), 7.12 (t, J=7.95 Hz, 1H), 6.74 (d, J=7.82 Hz, 1H), 6.61-6.68 (m, 2H), 5.96-5.85 (m, 1H), 4.00-4.19 (m, 1H), 3.72-3.80 (m, 1H), 2.36 (s, 3H), 1.75-1.82 (m, 1H), 0.75-0.92 (m, 2H), 0.57-0.66 (m, 2H)

¹⁹F NMR (377 MHz, CDCl₃) δ ppm 187.91 (s, 1 F) LCMS (Method B): retention time 1.80 min, 499 (M+H)

### Example P12: This Example Illustrates the Preparation of 2-chloro-6-(3-cyclopropylphenoxy)-N-[2-(2,4-dichlorophenyl)-2-fluoro-ethyl]pyrazolo[1,5-a]pyrimidine-7-carboxamide (Compound P-1.12, Table T1)

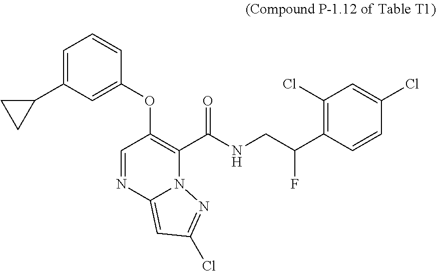 [SMILES: O=C(NCC(F)c1ccc(Cl)cc1Cl)c1c(Oc2cccc(C3CC3)c2)cnc2cc(Cl)nn12]

Ethyl 2-(3-cyclopropylphenoxy)-3-(dimethylamino)prop-2-enoate was prepared as described in example 1, steps a) and b).
a) Preparation of 2-chloro-6-(3-cyclopropylphenoxy)-4H-pyrazolo[1,5-a]pyrimidin-7-one

In a single-neck round bottom flask, a mixture of ethyl 2-(3-cyclopropylphenoxy)-3-(dimethylamino)prop-2-enoate (0.50 g, 2.00 mmol), 5-chloro-3H-pyrazol-3-amine (0.20 g, 2.00 mmol) and sodium acetate (0.20 g, 2.00 mmol) in acetic acid (10 mL) was stirred at 110° C. for 4 hr. Progress of the reaction was monitored by LCMS. After completion of the reaction, the reaction mixture was concentrated under reduced pressure and water was added. The solid obtained was filtered and dried to afford 2-chloro-6-(3-cyclopropylphenoxy)-4H-pyrazolo[1,5-a]pyrimidin-7-one as a brown solid.

LCMS (Method B): retention time 1.38 min, 302 (M+H)

### b) Preparation of 2,7-dichloro-6-(3-cyclopropylphenoxy)pyrazolo[1,5-a]pyrimidine

In a single-neck round bottom flask, a mixture of 2-chloro-6-(3-cyclopropylphenoxy)-4H-pyrazolo[1,5-a]pyrimidin-7-one (0.20 g, 0.66 mmol), pyridine (0.2 mL, 2.65 mmol) in phosphorus(V) oxychloride (5.0 mL, 50.3 mmol) was stirred at 90° C. for 4 hr. Progress of the reaction was monitored by LCMS. After completion of the reaction, the reaction mixture was diluted with ice water and extracted with EtOAc. The resulting organic layer was washed with brine, dried over sodium sulphate, and concentrated under reduced pressure to obtain a crude residue. The resulting crude residue was purified by silica gel chromatography (cyclohexane/ethylacetate) to afford 2,7-dichloro-6-(3-cyclopropylphenoxy)pyrazolo[1,5-a]pyrimidine as a yellow solid.

LCMS (Method B): retention time 1.77 min, 320 (M+H)

### c) Preparation of methyl 2-chloro-6-(3-cyclopropylphenoxy)pyrazolo[1,5-a]pyrimidine-7-carboxylate

An autoclave vessel charged with 2,7-dichloro-6-(3-cyclopropylphenoxy)pyrazolo[1,5-a]pyrimidine (0.08 g, 0.25 mmol), triethylamine (0.07 mL, 0.50 mmol), Pd(dppf)Cl₂CH₂Cl₂ (0.051 g, 0.06 mmol) in ethanol (20 mL). The reactor was flushed three times with carbon monoxide gas and loaded with 10 bar of carbon monoxide. The reaction mixture was heated to 80° C. for 2 hr. Progress of the reaction was monitored by LCMS. After completion of the reaction, the reaction mixture was diluted with water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford methyl 2-chloro-6-(3-cyclopropylphenoxy)pyrazolo[1,5-a]pyrimidine-7-carboxylate as a yellow gummy mass.

LCMS (Method B): retention time 2.92 min, 344 (M+H)

### d) Preparation of 2-chloro-6-(3-cyclopropylphenoxy)-N-[2-(2,4-dichlorophenyl)-2-fluoro-ethyl]pyrazolo[1,5-a]pyrimidine-7-carboxamide (Compound P-1.12, Table T1)

In a microwave vial, a mixture of methyl 2-chloro-6-(3-cyclopropylphenoxy)pyrazolo[1,5-a]pyrimidine-7-carboxylate (0.06 g, 0.14 mmol) and 2-(2,4-dichlorophenyl)-2-fluoro-ethanamine (0.029 g, 0.14 mmol) was stirred at 80° C. for 2 hr. The reaction progress was monitored by TLC and LCMS. After completion, the reaction mixture was diluted with water extracted with EtOAc. The resulting organic layer was washed with brine, dried over sodium sulphate, and concentrated under reduced pressure The resulting crude residue was purified by silica gel chromatography (cyclohexane/EtOAc) to afford 2-chloro-6-(3-cyclopropylphenoxy)-N-[2-(2,4-dichlorophenyl)-2-fluoro-ethyl]pyrazolo[1,5-a]pyrimidine-7-carboxamide as a brown solid.

¹H NMR (400 MHz, CDCl₃) δ ppm 8.72 (brs, 1H), 8.43 (s, 1H), 7.51 (d, J=8.38 Hz, 1H), 7.35-7.44 (m, 1H), 7.28-7.29 (m, 1H), 7.20-7.24 (m, 1H), 6.84-6.86 (d, J=7.75 Hz, 1H), 6.79 (s, 1H), 6.73-6.77 (m, 2H), 5.90-6.05 (m, 1H), 4.08-4.25 (m, 1H), 3.77-3.93 (m, 1H), 1.87 (tt, J=8.44, 5.00 Hz, 1H), 0.97-0.99 (m, 2H), 0.63-0.75 (m, 2H).

LCMS (Method B): retention time 1.88 min, 519 (M+H)

### Example P13: This Example Illustrates the Preparation of 6-(3-cyclopropylphenoxy)-N-[2-(2,4-dichlorophenyl)-2-fluoro-ethyl]-2-methyl-pyrazolo[1,5-a]pyrimidine-7-carboxamide (Compound P-1.13, Table T1)

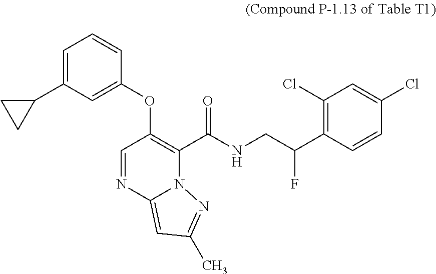 [SMILES: Cc1cc2ncc(Oc3cccc(C4CC4)c3)c(C(=O)NCC(F)c3ccc(Cl)cc3Cl)n2n1]

Ethyl 2-(3-cyclopropylphenoxy)-3-(dimethylamino)prop-2-enoate was prepared as described in example 1, steps a) and b).

### a) Preparation of 6-(3-cyclopropylphenoxy)-2-methyl-4H-pyrazolo[1,5-a]pyrimidin-7-one

In a single-neck round-bottom flask, a mixture of ethyl 2-(3-cyclopropylphenoxy)-3-(dimethylamino)prop-2-enoate (2.20 g, 7.59 mmol), 5-methyl-1H-pyrazol-3-amine (0.73 g, 7.59 mmol) and sodium acetate (0.30 g, 3.63 mmol) in acetic acid (22.0 mL) was stirred at 100° C. for 16 hr. Progress of the reaction was monitored by LCMS. After completion of the reaction, the reaction mixture was concentrated under reduced pressure, co-distilled twice with toluene under reduced pressure to obtain a crude residue. The resulting crude residue was purified by silica gel chromatography (cyclohexane/EtOAc) to afford 6-(3-cyclopropylphenoxy)-2-methyl-4H-pyrazolo[1,5-a]pyrimidin-7-one as a brown solid.

LCMS (Method B): retention time 1.28 min, 282 (M+H)

### b) Preparation of 7-chloro-6-(3-cyclopropylphenoxy)-2-methyl-pyrazolo[1,5-a]pyrimidine

In a single-neck round-bottom flask, a mixture of 6-(3-cyclopropylphenoxy)-2-methyl-4H-pyrazolo[1,5-a]pyrimidin-7-one (0.32 g, 0.79 mmol) and phosphorus(V) oxychloride (9.46 g, 60.5 mmol) was cooled to 0° C., and pyridine (0.25 g, 3.18 mmol) was added dropwise via syringe at 0° C. Resulting reaction mixture was stirred at 70° C. for 5 hr. Progress of the reaction was monitored by LCMS. After completion of the reaction, the reaction mixture was diluted with ice water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford 7-chloro-6-(3-cyclopropylphenoxy)-2-methyl-pyrazolo[1,5-a]pyrimidine as a pale yellow gummy mass.

LCMS (Method D): retention time 1.14 min, 300 (M+H)

### c) Preparation of methyl 6-(3-cyclopropylphenoxy)-2-methyl-pyrazolo[1,5-a]pyrimidine-7-carboxylate

An autoclave vessel charged with 7-chloro-6-(3-cyclopropylphenoxy)-2-methyl-pyrazolo[1,5-a]pyrimidine (0.19 g, 0.63 mmol), triethylamine (0.18 mL, 1.27 mmol), Pd(dppf)Cl₂CH₂Cl₂ (0.13 g, 0.15 mmol) in methanol (19 mL). The reactor was flushed three times with carbon monoxide gas and loaded with 10 bar of carbon monoxide. The reaction mixture was heated to 80° C. for 3 hr. Progress of the reaction was monitored by LCMS. After completion of the reaction, the reaction mixture was diluted with water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford methyl 6-(3-cyclopropylphenoxy)-2-methyl-pyrazolo[1,5-a]pyrimidine-7-carboxylate as a pale yellow gummy mass.

LCMS (Method C): retention time 1.18 min, 324 (M+H)

### d) Preparation of 6-(3-cyclopropylphenoxy)-N-[2-(2,4-dichlorophenyl)-2-fluoro-ethyl]-2-methyl-pyrazolo[1,5-a]pyrimidine-7-carboxamide (Compound P-1.13, Table T1

A 10 mL microwave vial was charged with methyl 6-(3-cyclopropylphenoxy)-2-methyl-pyrazolo[1,5-a]pyrimidine-7-carboxylate (70.0 mg, 0.22 mmol) and 2-(2,4-dichlorophenyl)-2-fluoro-ethanamine (90.0 mg, 0.43 mmol) and the vial was sealed and irradiated with microwave under stirring at 100° C. for 4 hr. The progress of the reaction was monitored by TLC and LCMS. After completion, the reaction mixture was diluted with water, and extracted with EtOAc. The resulting organic layer was washed with brine, dried over sodium sulphate, and concentrated under reduced pressure. The resulting crude residue was purified by silica gel chromatography (cyclohexane/EtOAc as an eluent) to afford 6-(3-cyclopropylphenoxy)-N-[2-(2,4-dichlorophenyl)-2-fluoro-ethyl]-2-methyl-pyrazolo[1,5-a]pyrimidine-7-carboxamide as an off-white solid.

¹H NMR (400 MHz, CDCl₃) δ ppm 9.87 (br s, 1H), 8.37 (s, 1H), 7.49 (d, J=8.50 Hz, 1H), 7.37-7.42 (m, 1H), 7.25-7.27 (m, 1H), 7.20 (t, J=7.94 Hz, 1H), 6.81 (d, J=7.75 Hz, 1H), 6.68-6.75 (m, 2H), 6.64 (s, 1H), 5.93-6.04 (dd, J=7.00, 3.25 Hz, 1H), 4.13-4.26 (m, 1H), 3.76-3.88 (m, 1H), 2.56 (s, 3H), 1.86 (tt, J=8.44, 5.00 Hz, 1H), 0.87-1.01 (m, 2H), 0.66-0.71 (m, 2H).

LCMS (Method C): retention time 1.30 min, 499 (M+H)

### Example P14: This Example Illustrates the Preparation of 6-(3-cyclopropylphenoxy)-N-[2-(2,4-dichlorophenyl)-2-fluoro-ethyl]-2-methyl-[1,2,4]triazolo[1,5-a]pyrimidine-7-carboxamide (Compound P-1.14, Table T1)

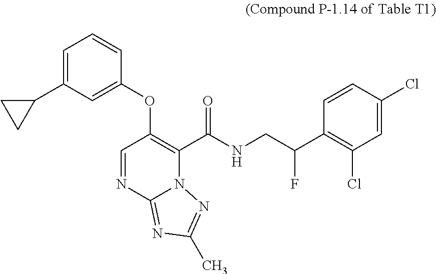 [SMILES: Cc1nc2ncc(Oc3cccc(C4CC4)c3)c(C(=O)NCC(F)c3ccc(Cl)cc3Cl)n2n1]

Note: ethyl 2-(3-cyclopropylphenoxy)-3-(dimethylamino)prop-2-enoate was prepared as described in example 1, step a) and b)

### a) Preparation of 6-(3-cyclopropylphenoxy)-2-methyl-4H-[1,2,4]triazolo[1,5-a]pyrimidin-7-one

In a single-neck round-bottom flask, a mixture of ethyl 2-(3-cyclopropylphenoxy)-3-(dimethylamino)prop-2-enoate (1.04 g, 3.59 mmol), 5-methyl-4H-1,2,4-triazol-3-amine (0.35 g, 3.59 mmol) and sodium acetate (0.30 g, 3.59 mmol) in acetic acid (10 mL) was stirred at 120° C. for 12 hr. The progress of the reaction was monitored by LCMS. After completion of the reaction, the reaction mixture was diluted with ice cold water and stirred for 15 min. The solid obtained was filtered over a Buchner funnel and dried under vacuum to afford 6-(3-cyclopropylphenoxy)-2-methyl-4H-[1,2,4]triazolo[1,5-a]pyrimidin-7-one as a white solid.

LCMS (Method B): retention time 1.20 min, 283 (M+H)

### b) Preparation of 7-chloro-6-(3-cyclopropylphenoxy)-2-methyl-[1,2,4]triazolo[1,5-a]pyrimidine 1

In a single-neck round-bottom flask, a mixture of 6-(3-cyclopropylphenoxy)-2-methyl-4H-[1,2,4]triazolo[1,5-a]pyrimidin-7-one (1.70 g, 6.02 mmol), N,N-dimethylaniline (3.15 g, 24.7 mmol) and phosphorus(V) oxychloride (42.8 mL, 458 mmol) was stirred at 90° C. for 12 hr. Progress of the reaction was monitored by LCMS. After completion of the reaction, the reaction mixture was diluted with ice water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford 7-chloro-6-(3-cyclopropylphenoxy)-2-methyl-[1,2,4]triazolo[1,5-a]pyrimidine] as a yellow solid.

LCMS (Method B): retention time 1.54 min, 301 (M+H)

### c) Preparation of methyl 6-(3-cyclopropylphenoxy)-2-methyl-[1,2,4]triazolo[1,5-alpyrimidine-7-carboxylate

An autoclave vessel was charged with 7-chloro-6-(3-cyclopropylphenoxy)-2-methyl-[1,2,4]triazolo[1,5-a]pyrimidine](0.53 g, 1.76 mmol), triethylamine (0.50 mL, 3.52 mmol), and Pd(dppf)Cl₂CH₂Cl₂ (0.36 g, 0.44 mmol) in methanol (20 mL). The reactor was flushed three times with carbon monoxide gas and loaded with 10 bar of carbon monoxide. The reaction mixture was heated to 80° C. for 2 hr. Progress of the reaction was monitored by LCMS. After completion of the reaction, the reaction mixture was diluted with water and extracted with EtOAc. The resulting organic layer was washed with brine, dried over sodium sulphate, and concentrated under reduced pressure to obtain a crude residue. The resulting crude residue was purified by silica gel chromatography (cyclohexane/EtOAc) to afford methyl 6-(3-cyclopropylphenoxy)-2-methyl-[1,2,4]triazolo[1,5-a]pyrimidine-7-carboxylate as a yellow gummy mass.

LCMS (Method B): retention time 1.42 min, 325 (M+H)

### d) Preparation of 6-(3-cyclopropylphenoxy)-N-[2-(2,4-dichlorophenyl)-2-fluoro-ethyl]-2-methyl-[1,2,4]triazolo[1,5-a]pyrimidine-7-carboxamide (Compound P-1.14, Table T1)

In a microwave vial, a mixture of methyl 6-(3-cyclopropylphenoxy)-2-methyl-[1,2,4]triazolo[1,5-a]pyrimidine-7-carboxylate (0.10 g, 0.30 mmol) and 2-(2,4-dichlorophenyl)-2-fluoro-ethanamine (0.12 g, 0.61 mmol) was stirred at 80° C. for 2 hr. Progress of the reaction was monitored by TLC and LCMS. After completion, the reaction mixture was diluted with water, extracted with EtOAc. The resulting organic layer was washed with brine, dried over sodium sulphate, and concentrated under reduced pressure. The resulting crude residue was purified by reverse phase chromatography (acetonitrile/water) to 6-(3-cyclopropylphenoxy)-N-[2-(2,4-dichlorophenyl)-2-fluoro-ethyl]-2-methyl-[1,2,4]triazolo[1,5-a]pyrimidine-7-carboxamide as an off-white solid.

¹H NMR (400 MHz, CDCl₃) δ ppm 9.12-9.0 (m, 1H), 8.69 (s, 1H), 7.46 (brd, J=8.19 Hz, 1H), 7.40 (s, 1H), 7.22-7.25 (m, 2H), 6.88 (d, J=7.58 Hz, 1H), 6.72-6.80 (m, 2H), 6.04-5.92 (m, 1H), 4.13-4.26 (m, 1H), 3.77-3.91 (m, 1H), 2.70 (s, 3H), 1.80-1.90 (m, 1H), 0.94-1.03 (m, 2H), 0.67-0.73 (m, 2H) LCMS (Method C): retention time 1.22 min, 500 (M+H)

### Example P15: This Example Illustrates the Preparation of 5-(3-cyclopropylphenoxy)-N-[2-(2,4-dichlorophenyl)-2-fluoro-ethyl]pyrazolo[1,5-b]pyridazine-4-carboxamide (Compound P-1.15 of Table T1)

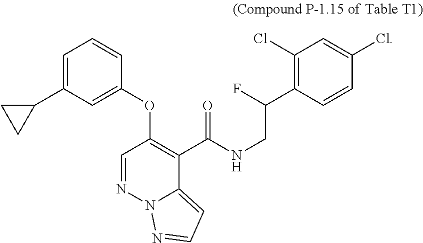 [SMILES: O=C(NCC(F)c1ccc(Cl)cc1Cl)c1c(Oc2cccc(C3CC3)c2)cnn2nccc12]

### a) Preparation of 2-[(3-iodopyrazol-1-yl)methoxy]ethyl-trimethyl-silane

To a solution of 3-iodo-1H-pyrazole (5.0 g, 25.8 mmol) in tetrahydrofuran (50 mL) at 0° C. was added sodium hydride (1.24 g, 30.9 mmol, 60 mass %). The reaction mixture was stirred for 15 min, then 2-(trimethylsilyl)ethoxymethyl chloride (5.28 mL, 28.4 mmol) was added to this at 0° C. The resulting reaction mixture was stirred at rt for 12 hr. The progress of the reaction was monitored by TLC and LCMS. After completion, the reaction mixture was diluted with water, extracted with EtOAc and the organic layer was washed with brine, dried over sodium sulphate, and concentrated under reduced pressure. The resulting crude residue was purified by silica gel chromatography (cyclohexane/EtOAc) to afford 2-[(3-iodopyrazol-1-yl)methoxy]ethyl-trimethyl-silane as a gummy mass.

LCMS (Method B): retention time 1.59 min, 325 (M+H)

### b) Preparation of 2-(3-cyclopropylphenoxy)-1-[1-(2-trimethylsilylethoxymethyl)pyrazol-3-yl] ethanone

In a two-neck round-bottom flask, 2-[(3-iodopyrazol-1-yl)methoxy]ethyl-trimethyl-silane (0.55 g., 1.70 mmol) was dissolved in tetrahydrofuran (3.4 mL) and cooled to 0° C. To this, isopropylmagnesium chloride lithium chloride complex solution (1.3 mol/L in THF, 1.3 mL, 1.70 mmol) was added dropwise and stirred for 30 minutes. After 30 minutes, 2-(3-cyclopropylphenoxy)-N-methoxy-N-methyl-acetamide (prepared separately in three steps) (0.20 g, 0.85 mmol) was added as a solution in tetrahydrofuran. The reaction mixture was stirred at rt for 2 hr. The progress of the reaction was monitored by TLC and LCMS. After completion, the reaction mixture was poured over ice cold water and acidified with 2N Hydrochloric acid, and extracted with EtOAc. The organic layer was washed with brine, dried over sodium sulphate, and concentrated under reduced pressure. The resulting crude residue was purified by silica gel chromatography (cyclohexane/EtOAc) to afford 2-(3-cyclopropylphenoxy)-1-[1-(2-trimethylsilylethoxymethyl)pyrazol-3-yl]ethanone as a yellow gummy mass.

LCMS (Method B): retention time 1.69 min, 373 (M+H)

### c) Preparation of 2-(3-cyclopropylphenoxy)-N-methoxy-N-methyl-acetamide

### i. Preparation of ethyl 2-(3-cyclopropylphenoxy)acetate

In a single-neck round-bottom flask, 3-cyclopropylphenol (2.0 g, 14.9 mmol) was dissolved in acetonitrile (22 mL) and to this caesium carbonate (5.82 g, 17.8 mmol) was added. To this, ethyl 2-bromoacetate (2.98 g, 17.8 mmol) was added and the resulting reaction mixture was stirred at rt for 4 hr. The progress of the reaction was monitored by LCMS. After completion of the reaction, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford ethyl 2-(3-cyclopropylphenoxy)acetate.

LCMS (Method B): retention time 1.48 min, 221 (M+H)

### ii. Preparation of 2-(3-cyclopropylphenoxy)acetic acid

To a solution of ethyl 2-(3-cyclopropylphenoxy)acetate (0.5 g, 2.27 mmol) in tetrahydrofuran (5.7 mL) and water (2.7 mL) was added lithium hydroxide (0.22 g, 9.08 mmol). The reaction mixture was stirred at rt for 2 hr. The progress of the reaction was monitored by TLC and LCMS. After completion of the reaction, the reaction mixture was diluted with water and washed with EtOAc. The aqueous layer was then acidified with 1N HCl and extracted with EtOAc. The combined organic layer were dried over sodium sulphate, and concentrated in vacuum to obtain 2-(3-cyclopropylphenoxy)acetic acid as a beige solid.

LCMS (Method B): retention time 1.28 min, 193 (M+H)

### iii. Preparation of 2-(3-cyclopropylphenoxy)-N-methoxy-N-methyl-acetamide

To 2-(3-cyclopropylphenoxy)acetic acid (1.34 g, 6.62 mmol) in EtOAc (26.8 mL), methoxy(methyl)ammonium chloride (0.97 g, 9.93 mmol) was added, followed by addition of 1-propanephosphonic anhydride solution (T3P, 50% in EtOAc, 4.64 g, 7.29 mmol) and N,N-diisopropylethylamine (3.5 mL, 19.9 mmol). The reaction mixture was stirred at rt for 12 hr. The progress of the reaction was monitored by TLC and LCMS. After completion, the reaction mixture was diluted with water, and extracted with EtOAc. Then the organic layer was washed with brine, dried over sodium sulphate, and concentrated under reduced pressure. The resulting crude residue was purified by silica gel chromatography (10-30% EtOAc in cyclohexane) to afford 2-(3-cyclopropylphenoxy)-N-methoxy-N-methyl-acetamide as a gummy mass.

LCMS (Method B): retention time 1.32 min, 236 (M+H)

### d) Preparation of 2-(3-cyclopropylphenoxy)-1-(1H-pyrazol-3-yl)ethanone

In a round-bottom flask, 2-(3-cyclopropylphenoxy)-1-[1-(2-trimethylsilylethoxymethyl)pyrazol-3-yl]ethanone (1.00 g, 2.70 mmol) was dissolved in methanol (11 mL) and to this 4M hydrochloric acid in dioxane (8.10 mL, 32 mmol) was added. The reaction mixture was stirred at 80° C. for 2 hr. The progress of the reaction was monitored by TLC and LCMS. After completion of the reaction, the reaction mixture was concentrated under reduced pressure, neutralised with saturated sodium bicarbonate and extracted with EtOAc. The combined organic layer was dried under reduced pressure to afford 2-(3-cyclopropylphenoxy)-1-(1H-pyrazol-3-yl)ethanone. The resulting crude residue was used as such for the next step.

LCMS (Method B): retention time 1.35 min, 241 (M−H)

### e) Preparation of 2-(3-cyclopropylphenoxy)-3-(dimethylamino)-1-(1H-pyrazol-3-yl)prop-2-en-1-one

In a sealed glass reactor, 2-(3-cyclopropylphenoxy)-1-(1H-pyrazol-3-yl)ethanone (0.80 g, 3.30 mmol) and 1-tert-butoxy-N,N,N′,N′-tetramethyl-methanediamine (7.58 mL, 33.0 mmol) solution was heated to 90° C. for 6 hr. The progress of the reaction was monitored by LCMS. After completion of the reaction, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford 2-(3-cyclopropylphenoxy)-3-(dimethylamino)-1-(1H-pyrazol-3-yl)prop-2-en-1-one as an off-white solid LCMS (Method B): retention time 1.30 min, 298 (M+H)

### f) Preparation of 5-(3-cyclopropylphenoxy)-7H-pyrazolo[1,5-b]pyridazin-4-one

In a round-bottom flask, 2-(3-cyclopropylphenoxy)-3-(dimethylamino)-1-(1H-pyrazol-3-yl)prop-2-en-1-one (0.06 g, 2.01 mmol) was dissolved in N-methyl-2-pyrrolidone (6 mL) and to this, potassium tert-butoxide (0.24 g, 2.12 mmol) was added as a solution in N-methyl-2-pyrrolidone (2.0 mL). The resulting reaction mixture was stirred at rt for 30 minutes. A solution of amino 4-nitrobenzoate (0.44 g, 2.42 mmol) in N-Methyl-2-pyrrolidone (2 mL) was added to this and reaction mixture was stirred at rt for overnight. The progress of the reaction was monitored by LCMS. After completion of the reaction, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford a mixture of 2-(3-cyclopropylphenoxy)-3-(dimethylamino)-1-(1H-pyrazol-3-yl)prop-2-en-1-one and 5-(3-cyclopropylphenoxy)-7H-pyrazolo[1,5-b]pyridazin-4-one. The mixture was taken as such for the next step.

LCMS (Method C): retention time 2.76 min, 298 (M+H) and retention time 2.95 min, 268 (M+H)

q) Preparation of 4-chloro-5-(3-cyclopropylphenoxy)pyrazolo[1,5-b]pvridazine In a round-bottom flask, 5-(3-cyclopropylphenoxy)-7H-pyrazolo[1,5-b]pyridazin-4-one (0.37 g, 1.38 mmol), pyridine (0.45 mL, 5.53 mmol) and phosphorus(V) oxychloride (9.8 mL, 105 mmol) were stirred at 90° C. for 12 hr. The progress of the reaction was monitored by LCMS. After completion of the reaction, the reaction mixture was concentrated. The crude reaction mixture was diluted with ice water, basified with saturated sodium bicarbonate, and extracted with EtOAc. The resulting organic layer was washed with brine, dried over sodium sulphate, and concentrated under reduced pressure to obtain a crude residue.

The resulting crude residue was purified by silica gel chromatography (cyclohexane/EtOAc) to afford 4-chloro-5-(3-cyclopropylphenoxy)pyrazolo[1,5-b]pyridazine as a brown gummy mass.

LCMS (Method B): retention time 1.57 min, 286 (M+H)

### h) Preparation of methyl 5-(3-cyclopropylphenoxy)pyrazolo[1,5-b]pyridazine-4-carboxylate

An autoclave vessel charged with 4-chloro-5-(3-cyclopropylphenoxy)pyrazolo[1,5-b]pyridazine (45.0 mg, 0.16 mmol), triethylamine (0.04 mL, 0.31 mmol), Pd(dppf)Cl₂CH₂Cl₂ (0.10 g, 0.13 mmol) in methanol (20 mL). The reactor was flushed three times with carbon monoxide gas and loaded with 10 bar of carbon monoxide. The reaction mixture was heated to 90° C. for 6 hr. The progress of the reaction was monitored by LCMS. After completion of the reaction, the reaction mixture was diluted with water and extracted with EtOAc. The organic layer was washed with brine, dried over sodium sulphate, and concentrated under reduced pressure to obtain a crude residue. The resulting crude residue was purified by silica gel chromatography (cyclohexane/EtOAc) to afford methyl 5-(3-cyclopropylphenoxy)pyrazolo[1,5-b]pyridazine-4-carboxylate as a yellow gummy mass.

LCMS (Method B): retention time 1.48 min, 310 (M+H)

### i) Preparation of lithium; 5-(3-cyclopropylphenoxy)pyrazolo[1,5-b]pyridazine-4-carboxylate

To a solution of methyl 5-(3-cyclopropylphenoxy)pyrazolo[1,5-b]pyridazine-4-carboxylate (0.03 g, 0.10 mmol) in tetrahydrofuran (1 mL) and water (0.06 mL) was added lithium hydroxide monohydrate (12.4 mg, 0.30 mmol). The reaction mixture was stirred at rt for 5 hr. The progress of the reaction was monitored by TLC and LCMS. After completion of the reaction, the reaction mixture was concentrated under reduced pressure at 30° C., followed twice by co-distillation with toluene to afford lithium 5-(3-cyclopropylphenoxy)pyrazolo[1,5-b]pyridazine-4-carboxylate which was used as such for the next step.

LCMS (Method B): retention time 1.34 min, 296 (M+H)

### i) Preparation of 5-(3-cyclopropylphenoxy)-N-[2-(2,4-dichlorophenyl)-2-fluoro-ethyl]pyrazolo[1,5-b]pyridazine-4-carboxamide (Compound P-1.15, Table T1)

To lithium 5-(3-cyclopropylphenoxy)pyrazolo[1,5-b]pyridazine-4-carboxylate (0.04 g, 0.13 mmol) in EtOAc (0.66 mL), 2-(2,4-dichlorophenyl)-2-fluoro-ethanamine (33.0 mg, 0.16 mmol) was added, followed by the addition of a 1-propanephosphonic anhydride solution (T3P, 50% in EtOAc, 0.23 mL, 0.40 mmol) and triethylamine (0.04 mL, 0.30 mmol) at 0° C. This reaction mixture was stirred at rt for 12 hr. The progress of the reaction was monitored by TLC and LCMS. After completion, the reaction mixture was diluted with water, extracted with EtOAc. The resulting organic layer was washed with brine, dried over sodium sulphate, and concentrated under reduced pressure. The resulting crude residue was purified by silica gel chromatography (cyclohexane/EtOAc) to afford 5-(3-cyclopropylphenoxy)-N-[2-(2,4-dichlorophenyl)-2-fluoro-ethyl]pyrazolo[1,5-b]pyridazine-4-carboxamide as a yellow gummy mass.

¹H NMR (400 MHz, C₀₀₁₃) b ppm 8.10 (d, J=2.38 Hz, 1H), 7.98 (s, 1H), 7.73 (br s, 1H), 7.31-7.39 (m, 2H), 7.29 (d, J=2.63 Hz, 2H), 7.10 (dd, J=8.38, 2.00 Hz, 1H), 7.08 (d, J=7.75 Hz, 1H), 6.86 (ddd, J=8.13, 2.50, 0.88 Hz, 1H), 6.76-6.81 (m, 1H), 5.89-6.02 (m, 1H), 3.86-4.16 (m, 2H), 1.89-1.97 (m, 1H), 0.98-1.13 (m, 2H), 0.69-0.78 (in, 2H) LCMS (Method B): retention time 1.64 min, 485 (M+H) Further examples of synthesized compounds of formula (I) are shown in Table T1.

TABLE T1
Physical data of compounds of formula (I)

| | | | Rt | Mass | Meth. |
|---|---|---|---|---|---|
| Entry | Name | Structure | (min) | charge | ** |
| P-1.1 | 6-(3-cyclopropylphenoxy)-N- [2-(2,4-dichlorophenyl)-2- fluoro-ethyl]imidazo[1,2- a]pyrimidine-5-carboxamide | 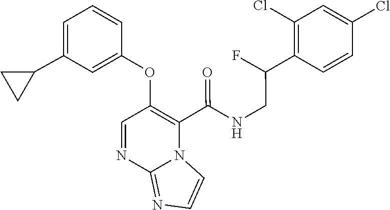 [SMILES: O=C(NCC(F)c1ccc(Cl)cc1Cl)c1c(Oc2cccc(C3CC3)c2)cnc2nccn12] | 1.55 | 485 | B |
| P-1.2 | 6-(3-cyclopropylphenoxy)-N- [2-(2,4-dichlorophenyl)-2- fluoro-ethyl]- [1,2,4]triazolo[1,5- a]pyrimidine-7-carboxamide | 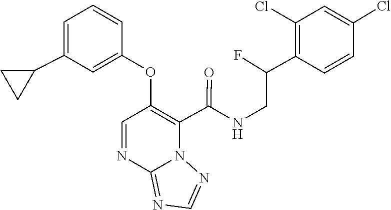 [SMILES: O=C(NCC(F)c1ccc(Cl)cc1Cl)c1c(Oc2cccc(C3CC3)c2)cnc2ncnn12] | 1.22 | 486 | C |
| P_1.3 | 6-(3-cyclopropylphenoxy)-N- [2-(2,4-dichlorophenyl)-2- fluoro-ethyl]pyrazolo[1,5- a]pyrimidine-7-carboxamide | 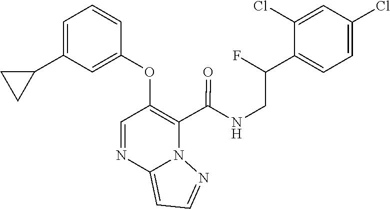 [SMILES: O=C(NCC(F)c1ccc(Cl)cc1Cl)c1c(Oc2cccc(C3CC3)c2)cnc2ccnn12] | 1.29 | 485 | C |
| P-1.4 | 7-(3-cyclopropylphenoxy)-N- [2-(2,4-dichlorophenyl)-2- fluoro-ethyl]imidazo[1,2- b]pyridazine-8-carboxamide | 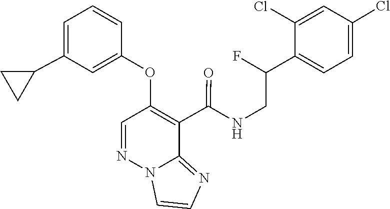 [SMILES: O=C(NCC(F)c1ccc(Cl)cc1Cl)c1c(Oc2cccc(C3CC3)c2)cnn2ccnc12] | 1.48 | 485 | B |
| P-1.5 | 6-(3-cyclopropylphenoxy)-N- [2-(2,4-dichlorophenyl)ethyl]- [1,2,4]triazolo[1,5- a]pyrimidine-7-carboxamide | 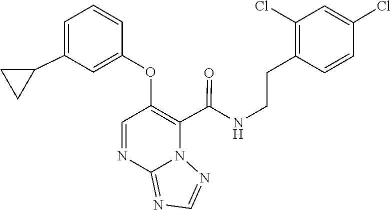 [SMILES: O=C(NCCc1ccc(Cl)cc1Cl)c1c(Oc2cccc(C3CC3)c2)cnc2ncnn12] | 1.24 | 468 | C |
| P-1.6 | 6-(3-cyclopropylphenoxy)-N- [2-(2,4- dichlorophenyl)ethyl]imidazo [1,2-a]pyrimidine-5- carboxamide | 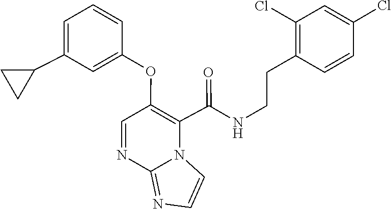 [SMILES: O=C(NCCc1ccc(Cl)cc1Cl)c1c(Oc2cccc(C3CC3)c2)cnc2nccn12] | 1.15 | 467 | D |
| P-1.7 | 6-(3-cyclopropylphenoxy)-N- [2-(2,4-dichlorophenyl)-2- fluoro-ethyl]-2-methyl- imidazo[1,2-a]pyrimidine-5- carboxamide | 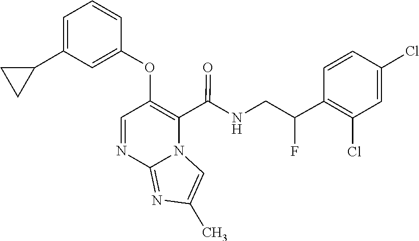 [SMILES: Cc1cn2c(C(=O)NCC(F)c3ccc(Cl)cc3Cl)c(Oc3cccc(C4CC4)c3)cnc2n1] | 1.22 | 499 | C |
| P-1.8 | 2-cyano-6-(3- cyclopropylphenoxy)-N-[2- (2,4-dichlorophenyl)-2-fluoro- ethyl]pyrazolo[1,5- a]pyrimidine-7-carboxamide | 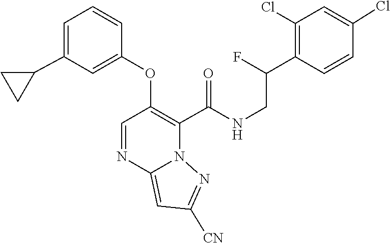 [SMILES: N#Cc1cc2ncc(Oc3cccc(C4CC4)c3)c(C(=O)NCC(F)c3ccc(Cl)cc3Cl)n2n1] | 1.19 | 510 | A |
| P-1.9 | 3-chloro-6-(3- cyclopropylphenoxy)-N-[2- (2,4-dichlorophenyl)-2-fluoro- ethyl]pyrazolo[1,5- a]pyrimidine-7-carboxamide | 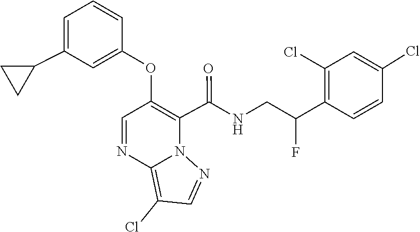 [SMILES: O=C(NCC(F)c1ccc(Cl)cc1Cl)c1c(Oc2cccc(C3CC3)c2)cnc2c(Cl)cnn12] | 1.25 | 517 | C |
| P-1.10 | 6-(3-cyclopropylphenoxy)-N- [2-(2,4-dichlorophenyl)-2- fluoro-ethyl]-2-fluoro- pyrazolo[1,5-a]pyrimidine-7- carboxamide | 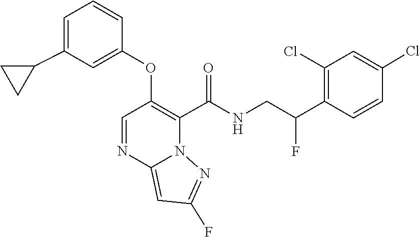 [SMILES: O=C(NCC(F)c1ccc(Cl)cc1Cl)c1c(Oc2cccc(C3CC3)c2)cnc2cc(F)nn12] | 1.29 | 503 | C |
| P-1.11 | 6-(3-cyclopropylphenoxy)-N- [2-(2,4-dichlorophenyl)-2- fluoro-ethyl]-3-methyl- pyrazolo[1,5-a]pyrimidine-7- carboxamide | 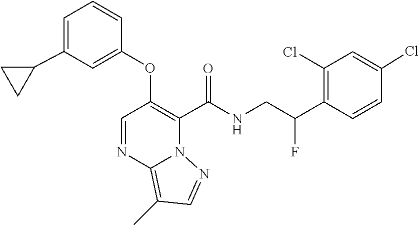 [SMILES: Cc1cnn2c(C(=O)NCC(F)c3ccc(Cl)cc3Cl)c(Oc3cccc(C4CC4)c3)cnc12] | 1.80 | 499 | B |
| P-1.12 | 2-chloro-6-(3- cyclopropylphenoxy)-N-[2- (2,4-dichlorophenyl)-2-fluoro- ethyl]pyrazolo[1,5- a]pyrimidine-7-carboxamide | 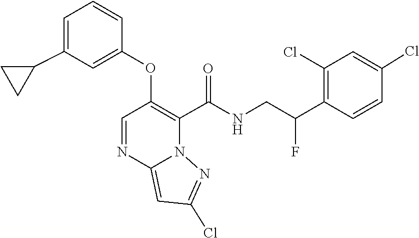 [SMILES: O=C(NCC(F)c1ccc(Cl)cc1Cl)c1c(Oc2cccc(C3CC3)c2)cnc2cc(Cl)nn12] | 1.88 | 519 | B |
| P-1.13 | 6-(3-cyclopropylphenoxy)-N- [2-(2,4-dichlorophenyl)-2- fluoro-ethyl]-2-methyl- pyrazolo[1,5-a]pyrimidine-7- carboxamide | 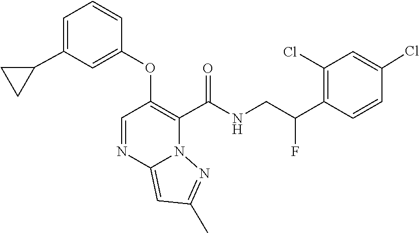 [SMILES: Cc1cc2ncc(Oc3cccc(C4CC4)c3)c(C(=O)NCC(F)c3ccc(Cl)cc3Cl)n2n1] | 1.30 | 499 | C |
| P-1.14 | 6-(3-cyclopropylphenoxy)-N- [2-(2,4-dichlorophenyl)-2- fluoro-ethyl]-2-methyl- [1,2,4]triazolo[1,5- a]pyrimidine-7-carboxamide | 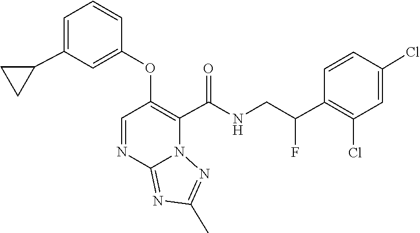 [SMILES: Cc1nc2ncc(Oc3cccc(C4CC4)c3)c(C(=O)NCC(F)c3ccc(Cl)cc3Cl)n2n1] | 1.22 | 500 | C |
| P-1.15 | 5-(3-cyclopropylphenoxy)-N- [2-(2,4-dichlorophenyl)-2- fluoro-ethyl]pyrazolo[1,5- b]pyridazine-4-carboxamide | 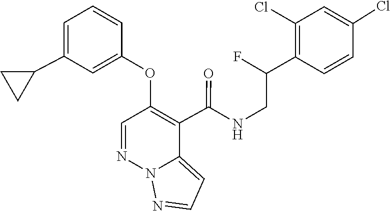 [SMILES: O=C(NCC(F)c1ccc(Cl)cc1Cl)c1c(Oc2cccc(C3CC3)c2)cnn2nccc12] | 1.64 | 485 | B |
| P-1.16 | 3-chloro-6-(3- cyclopropylphenoxy)-N-[(2R)- 2-(2,4-dichlorophenyl)-2- fluoro-ethyl]pyrazolo[1,5- a]pyrimidine-7-carboxamide | 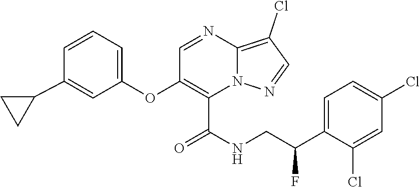 [SMILES: O=C(NC[C@H](F)c1ccc(Cl)cc1Cl)c1c(Oc2cccc(C3CC3)c2)cnc2c(Cl)cnn12] | 3.39 | 519 | E |
| P-1.17 | 3-chloro-6-(3- cyclopropylphenoxy)-N-[(2S)- 2-(2,4-dichlorophenyl)-2- fluoro-ethyl]pyrazolo[1,5- a]pyrimidine-7-carboxamide | 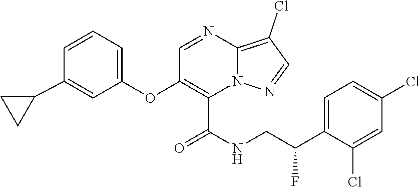 [SMILES: O=C(NC[C@@H](F)c1ccc(Cl)cc1Cl)c1c(Oc2cccc(C3CC3)c2)cnc2c(Cl)cnn12] | 5.59 | 519 | E |
| P-1.18 | 3-cyano-6-(3- cyclopropylphenoxy)-N-[2- (2,4-dichlorophenyl)-2-fluoro- ethyl]-2-methoxy- pyrazolo[1,5-a]pyrimidine-7- carboxamide | 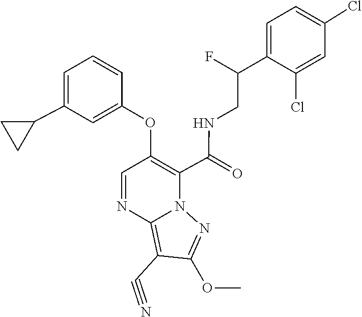 [SMILES: COc1nn2c(C(=O)NCC(F)c3ccc(Cl)cc3Cl)c(Oc3cccc(C4CC4)c3)cnc2c1C#N] | 1.21 | 540 | A |
| P-1.19 | 3-chloro-6-(3- cyclopropylphenoxy)-N-[2- (2,4-dichlorophenyl)-2-fluoro- ethyl]-2-methyl-pyrazolo[1,5- a]pyrimidine-7-carboxamide | 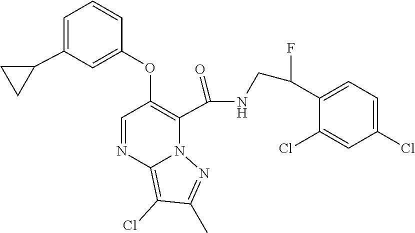 [SMILES: Cc1nn2c(C(=O)NCC(F)c3ccc(Cl)cc3Cl)c(Oc3cccc(C4CC4)c3)cnc2c1Cl] | 1.26 | 535 | C |
| P-1.20 | 3-cyano-6-(3- cyclopropylphenoxy)-N-[2- (2,4-dichlorophenyl)-2-fluoro- ethyl]-2-methyl-pyrazolo[1,5- a]pyrimidine-7-carboxamide | 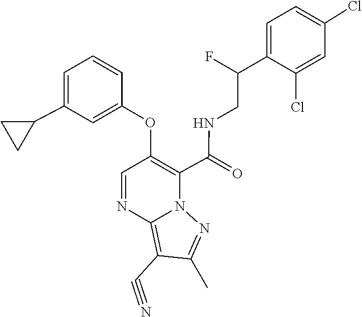 [SMILES: Cc1nn2c(C(=O)NCC(F)c3ccc(Cl)cc3Cl)c(Oc3cccc(C4CC4)c3)cnc2c1C#N] | 1.19 | 524 | A |

* [M + H] measured;
** Method used

### Biological Examples and Test Methods

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Compounds of Formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

### General Description of Test Methods

Leaf disks or leaf segments of various plant species are cut from plants grown in a greenhouse. The cut leaf disks or segments are placed in multiwell plates (24-well format) onto water agar. The leaf disks are sprayed with a test solution before (preventative) or after (curative) inoculation. Compounds to be tested are prepared as DMSO solutions (max. 10 mg/mL) which are diluted to the appropriate concentration with 0.025% Tween20 just before spraying. The inoculated leaf disks or segments are incubated under defined conditions (temperature, relative humidity, light, etc.) according to the respective test system. A single evaluation of disease level is carried out 3 to 14 days after inoculation, depending on the pathosystem. Percent disease control relative to the untreated check leaf disks or segments is then calculated.

Mycelia fragments or conidia suspensions of a fungus prepared either freshly from liquid cultures of the fungus or from cryogenic storage, are directly mixed into nutrient broth. DMSO solutions of the test compound (max. 10 mg/mL) are diluted with 0.025% Tween20 by a factor of 50 and 10 μL of this solution is pipetted into a microtiter plate (96-well format). The nutrient broth containing the fungal spores/mycelia fragments is then added to give an end concentration of the tested compound. The test plates are incubated in the dark at 24° C. and 96% relative humidity. The inhibition of fungal growth is determined photometrically after 2 to 7 days, depending on the pathosystem, and percent antifungal activity relative to the untreated check is calculated.

### Example B-1: Alternaria solani/Tomato/Leaf Disc (Early Blight)

Tomato leaf disks cv. Baby are placed on agar in multiwell plates (24-well format) and sprayed with the formulated test compound diluted in water. The leaf disks are inoculated with a spore suspension of the fungus 2 days after application. The inoculated leaf disks are incubated at 23° C./21° C. (day/night) and 80% rh under a light regime of 12/12 h (light/dark) in a climate cabinet and the activity of a compound is assessed as percent disease control compared to untreated when an appropriate level of disease damage appears on untreated check disk leaf disks (5-7 days after application).

The following compounds gave at least 80% control of Alternaria solani at 200 ppm when compared to untreated control under the same conditions, which showed extensive disease development:
P-1.13

### Example B-2: Botryotinia Fuckeliana (Botrytis cinerea)/Liquid Culture (Gray Mould)

Conidia of the fungus from cryogenic storage are directly mixed into nutrient broth (Vogels broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

The following compounds gave at least 80% control of Botryotinia fuckeliana at 20 ppm when compared to untreated control under the same conditions, which showed extensive disease development:
P-1.1, P-1.2, P-1.3, P-1.5, P-1.7, P-1.8, P-1.9, P-1.10, P-1.11, P-1.12, P-1.13, P-1.14, P-1.15, P-1.16, P-1.18, P-1.19, and P-1.20

### Example B-3: Glomerella Laaenarium (Colletotrichum Lagenarium)/Liquid Culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3-4 days after application.

The following compounds gave at least 80% control of Glomerella lagenarium at 20 ppm when compared to untreated control under the same conditions, which showed extensive disease development:
P-1.1, P-1.2, P-1.3, P-1.6, P-1.7, P-1.8, P-1.10, P-1.11, P-1.12, P-1.13, P-1.14, P-1.15, P-1.16, and P-1.20

### Example B-4: Blumeria Graminis f. Sp. Tritici (Erysiphe graminis f. Sp. Tritici)/Wheat/Leaf Disc Preventative (Powdery Mildew on Wheat)

Wheat leaf segments cv. Kanzler are placed on agar in a multiwell plate (24-well format) and sprayed with the formulated test compound diluted in water. The leaf disks are inoculated by shaking powdery mildew infected plants above the test plates 1 day after application. The inoculated leaf disks are incubated at 20° C. and 60% rh under a light regime of 24 h darkness followed by 12 h light/12 h darkness in a climate chamber and the activity of a compound is assessed as percent disease control compared to untreated when an appropriate level of disease damage appears on untreated check leaf segments (6-8 days after application).

The following compounds gave at least 80% control of Blumeria graminis f. sp. tritici at 200 ppm when compared to untreated control under the same conditions, which showed extensive disease development:
P-1.16

### Example B-5: Phaeosphaeria Nodorum (Septoria nodorum)/Wheat/Leaf Disc Preventative (Glume Blotch)

Wheat leaf segments cv. Kanzler are placed on agar in a multiwell plate (24-well format) and sprayed with the formulated test compound diluted in water. The leaf disks are inoculated with a spore suspension of the fungus 2 days after application. The inoculated test leaf disks are incubated at 20° C. and 75% rh under a light regime of 12 h light/12 h darkness in a climate cabinet and the activity of a compound is assessed as percent disease control compared to untreated when an appropriate level of disease damage appears in untreated check leaf disks (5-7 days after application).

The following compounds gave at least 80% control of Phaeosphaeria nodorum at 200 ppm when compared to untreated control under the same conditions, which showed extensive disease development:
P-1.16

### Example B-6: Monographella Nivalis (Microdochium Nivale)/Liquid Culture (Foot Rot Cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

The following compounds gave at least 80% control of Monographella nivalis at 20 ppm when compared to untreated control under the same conditions, which showed extensive disease development:
P-1.1, P-1.6, P-1.16, and P-1.20

### Example B-7: Phytophthora infestans/Tomato/Leaf Disc Preventative (Late Blight)

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

The following compounds gave at least 80% control of Phytophthora infestans at 200 ppm when compared to untreated control under the same conditions, which showed extensive disease development:
P-1.9

### Example B-8: Puccinia recondita f. Sp. Tritici/Wheat/Leaf Disc Curative (Brown Rust)

Wheat leaf segments cv. Kanzler are placed on agar in multiwell plates (24-well format). The leaf segments are inoculated with a spore suspension of the fungus. Plates are stored in darkness at 19° C. and 75% rh. The formulated test compound diluted in water is applied 1 day after inoculation. The leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (6-8 days after application).

The following compounds gave at least 80% control of Puccinia recondita f. sp. tritici at 200 ppm when compared to untreated control under the same conditions, which showed extensive disease development:
P-1.8

### Example B-9: Magnaporthe grisea (Pyricularia oryzae)/Rice/Leaf Disc Preventative (Rice Blast)

Rice leaf segments cv. Ballila are placed on agar in a multiwell plate (24-well format) and sprayed with the formulated test compound diluted in water. The leaf segments are inoculated with a spore suspension of the fungus 2 days after application. The inoculated leaf segments are incubated at 22° C. and 80% rh under a light regime of 24 h darkness followed by 12 h light/12 h darkness in a climate cabinet and the activity of a compound is assessed as percent disease control compared to untreated when an appropriate level of disease damage appears in untreated check leaf segments (5-7 days after application).

The following compounds gave at least 80% control of Magnaporthe grisea at 200 ppm when compared to untreated control under the same conditions, which showed extensive disease development:
P-1.9

### Example B-10: Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segmens are inoculated with a spore suspension of the fungus 2 days after application. The inoculated leaf segments are incubated at 20° C. and 65% rh under a light regime of 12 h light/12 h darkness in a climate cabinet and the activity of a compound is assessed as disease control compared to untreated when an appropriate level of disease damage appears in untreated check leaf segments (5-7 days after application).

The following compounds gave at least 80% control of Pyrenophora teres at 200 ppm when compared to untreated control under the same conditions, which showed extensive disease development:
P-1.13

### Example B-11: Sclerotinia sclerotiorum/Liquid Culture (Cottony Rot)

Mycelia fragments of a newly grown liquid culture of the fungus are directly mixed into nutrient broth (PDB potato dextrose broth). After placing a (DMSO) solution of test compound into a microtiter plate (96-well format) the nutrient broth containing the fungal material is added. The test plates are incubated at 24° C. and the inhibition of growth is determined photometrically 3-4 days after application.

The following compounds gave at least 80% control of Sclerotinia sclerotiorum at 20 ppm when compared to untreated control under the same conditions, which showed extensive disease development:
P-1.9, and P-1.16

1. A compound of formula (I) 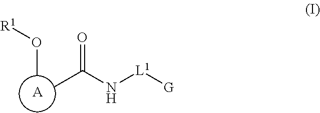
or a stereoisomer, enantiomer, salt, or N-oxide of the compound of formula (I), wherein:
R¹ is phenyl unsubstituted or substituted with 1, 2 or 3 independently selected substituents R¹¹; or
R¹ is a 5- or 6-membered monocyclic heteroaryl ring comprising 1, 2 or 3 heteroatoms each independently selected from N, O and S, wherein said heteroaryl ring is unsubstituted or substituted with 1 or 2 independently selected substituents R¹¹;
R¹¹ is hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, or cyclopropyloxy;
L^{r} represents a direct bond, —O—, or —O—C(R^{L1A})(R^{L1B})—; wherein R^{L1A} and R^{L1B} are independently selected from hydrogen or methyl; or R^{L1A} and R^{L1B} together with the carbon atom to which they are attached, form a cyclopropyl; or
L¹ represents —NR¹⁰—(CR²R³)ₘ—wherein R¹⁰ is selected from hydrogen or methyl; and m is 0 or 1; or
L¹ represents 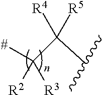
wherein # marks the bond to the nitrogen atom and the staggered line marks the bond to the group G;
R² and R³ are independently selected from hydrogen or methyl; and n is 0 or 1;
R⁴ and R⁵ are independently selected from hydrogen, hydroxy, fluoro, methyl, cyano, or methoxy; or
R⁴ and R⁵ together with the carbon atom to which they are attached, form a carbonyl, cyclopropyl, or cyclobutyl group;
G is selected from G-1, G-2, G-3, or G-4, wherein:
G-1 is phenyl or phenoxy, wherein said phenyl or phenoxy is unsubstituted or substituted with 1, 2 or 3 independently selected substituents R^{G1};
G-2 is a 5- or 6-membered monocyclic heteroaryl or heteroaryl-oxy; wherein said heteroaryl comprises 1, 2 or 3 heteroatoms each independently selected from N, O and S; and wherein said heteroaryl is unsubstituted or substituted with 1 or 2 independently selected substituents R^{G2};
G-3 is a 9- or 10-membered heterobicyclic ring system comprising 1, 2 or 3 heteroatoms each independently selected from N, O and S; wherein said heterobicyclic ring system is saturated, partially unsaturated, or aromatic; and wherein said heterobicyclic ring system is unsubstituted or substituted with 1 or 2 independently selected substituents R^{G3};
G-4 is a 9- or 10-membered carbobicyclic ring system; wherein said carbobicyclic ring system is saturated, partially unsaturated, or aromatic; and wherein said carbobicyclic ring system is unsubstituted or substituted with 1 or 2 independently selected substituents R^{G4};
R^{G1}, R^{G2}, R^{G3}, and R^{G4} are independently hydroxyl, halogen, mercapto, amino, cyano, methyl, ethyl, propyl, iso-propyl, vinyl, ethynyl, difluoromethyl, trifluoromethyl, methoxy, ethoxy, propyloxy, iso-propyloxy, tert-butoxy, propynoxy, methylsulfanyl, methylsulfonyl, difluoromethoxy, trifluoromethoxy, cyclopropyl, cyclobutyl, or cyclopropyloxy;
A is selected from A-1 to A-17: 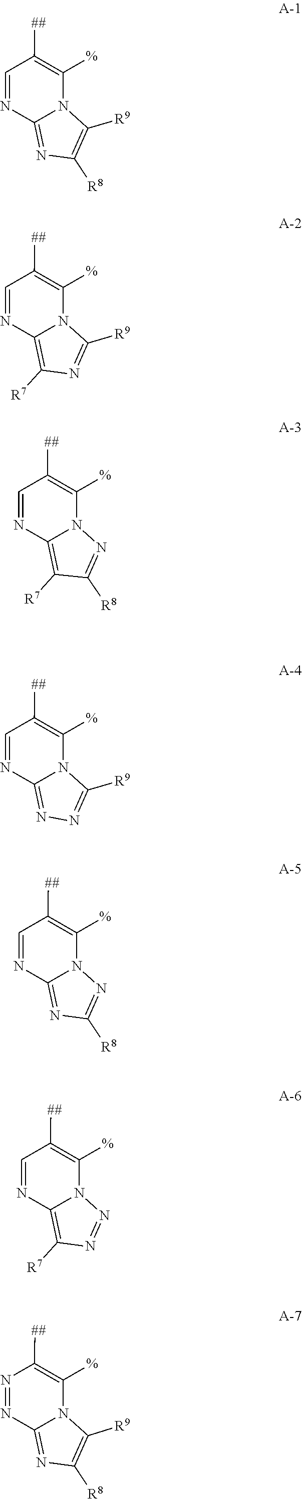 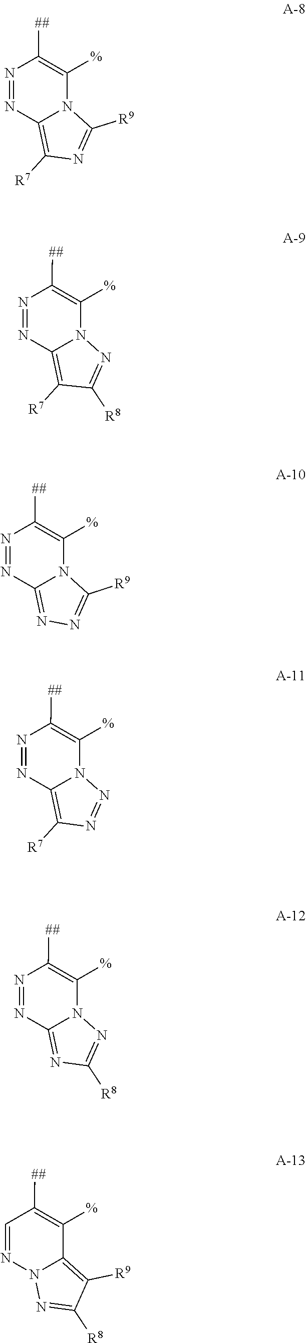 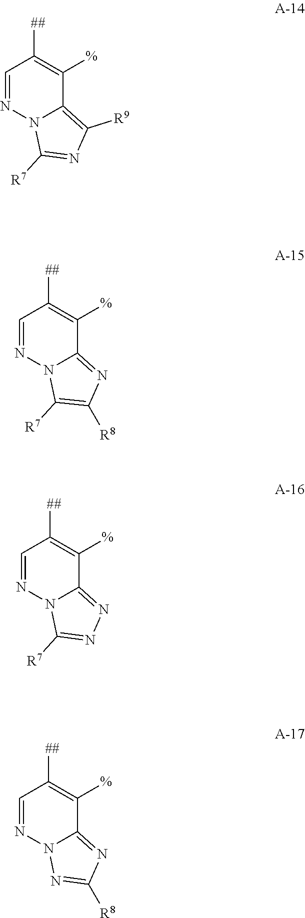
wherein ## marks the bond to —O—R¹; % marks the bond to —C(O)—N(H)-L¹-G; and
R⁷, R⁸, R⁹ are independently selected from hydrogen, fluoro, chloro, bromo, iodo, methyl, ethyl, propyl, isopropyl, allyl, propargyl, cyclopropylmethyl, cyclopentyl,
cyclohexyl, —C(═O)OCH₃, —C(═O)N(CH₃)₂, 2-(dimethylamino)-2-oxo-ethyl, 2-(methylamino)-2-oxo-ethyl, difluoromethyl, trifluoromethyl, methylsulfonyl, methylsulfanyl, methoxy, ethoxy, cyano, hydroxyl, mercapto, or amino.

2. The compound according to claim 1, wherein A is selected from A-1, A-3, A-5, A-13 and A-15, and wherein R⁷, R⁸ and R⁹ are independently selected from hydrogen, fluoro, chloro, methyl, cyclopropyl, or cyano.

3. The compound according to claim 1, wherein L¹ is a direct bond, —NR¹⁰—CR²R³—, or —CR²R³—CR⁴R⁵—, wherein:
R¹⁰ is selected from hydrogen or methyl;
R² and R³ are independently selected from hydrogen or methyl; and
R⁴ and R⁵ are independently selected from hydrogen, hydroxy, fluoro, methyl, cyano, or methoxy.

4. The compound according to claim 1, wherein R¹ is phenyl substituted with a single substituent selected from methyl or cyclopropyl.

5. The compound according to claim 1, wherein R¹ is pyridine substituted with a single substituent selected from chloro, cyano, or methyl.

6. The compound according to claim 1, wherein G is phenyl or phenoxy, unsubstituted or substituted with one or two substituents independently selected from chloro or methyl.

7. The compound according to claim 1, wherein G is pyridine substituted with one or two substituents independently selected from chloro or methyl.

8. An agrochemical composition comprising a fungicidally effective amount of a compound according to claim 1.

9. The composition according to claim 8, further comprising at least one additional active ingredient and/or an agrochemically-acceptable diluent or carrier.

10. A method of controlling or preventing infestation of useful plants by phytopathogenic microorganisms, wherein a fungicidally effective amount of a compound according to claim 1, or a composition comprising this compound as active ingredient, is applied to the plants, to parts thereof or the locus thereof.

11. Use of a compound according to claim 1 as a fungicide.

12. A plant propagation material, such as a seed, comprising, or treated with or adhered thereto, a compound as defined in claim 1, or a composition comprising this compound as active ingredient.

13. A compound of formula (II), (V), (VI), and (XVIII) 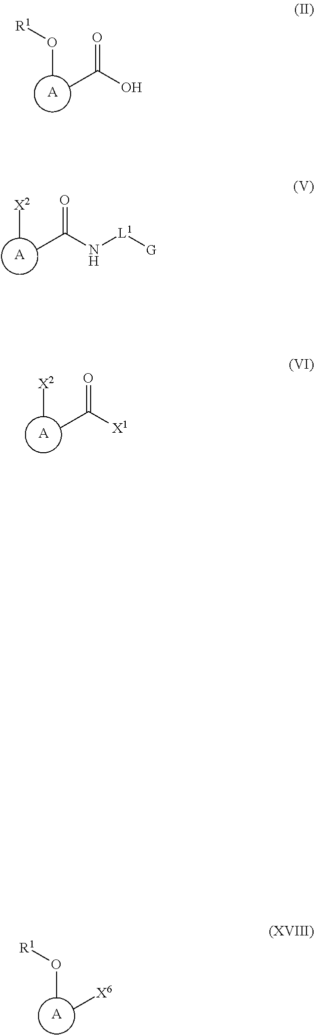
wherein
A, R¹, L¹ and G are as defined for compound of formula (I) in claim 1, and wherein:
X¹ is C₁-C₄-alkoxy;
X² is a suitable leaving group such as fluoro, chloro, bromo, iodo, BF₃K, B(OH)₂, or B(pinacol); and
X⁶ is chloro, bromo, iodo, or trifluoromethanesulfonyl-O—.
